(19) United States
(12) Patent Application Publication
(10) Pub. No.: US 2024/0225451 A1
(43) Pub. Date: Jul. 11, 2024

(54) RAMAN SPECTROSCOPY SYSTEM AND METHODS OF USING THE SAME

(71) Applicant: Board of Supervisors of Louisiana State University and Agricultural and Mechanical College, Baton, LA (US)

(72) Inventors: Michael E. Dunham, New Orleans, LA (US); Andrew J. McWhorter, Baton Rouge, LA (US); Jian Xu, Baton Rouge, LA (US); Jian Zhang, Baton Rouge, LA (US)

(21) Appl. No.: 18/030,943

(22) PCT Filed: Oct. 8, 2021
(86) PCT No.: PCT/US2021/054262

§ 371 Date: Apr. 7, 2023

Related U.S. Application Data
(60) Provisional application No. 63/089,322, filed on Oct. 8, 2020.
(60) Provisional application No. 63/234,062, filed on Aug. 17, 2021.

(51) Int. Cl. A61B 5/00; A61B 1/01; A61B 1/267; G01N 21/65

(52) U.S. Cl. CPC A61B 5/0075; A61B 1/01; A61B 1/267; A61B 5/7264; A61B 5/742; G01N 21/65

(57) ABSTRACT

Aspects of the invention are drawn to a Raman spectroscopy system that incorporates a tissue probe into a thin sheath that is adapted to fit any flexible or rigid laryngoscope. The Raman probe system can comprise a probe, a laser source, an excitation signal filter, a collection filter, a charge couple device detector, a signal collection system, a housing unit, a computer, a display, or a combination thereof. In certain embodiments, the signal collection system comprises a spectrum collection range of about 200 cm-1 to about 4000 cm.

This application claims priority from U.S. Provisional Application No. 63/089,322 filed on Oct. 8, 2020, and U.S. Provisional Application No. 63/234,062 filed on Aug. 17, 2021, the contents of each of which are incorporated herein by reference in their entireties.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

Aspects of the invention are drawn to a Raman spectroscopy system that incorporates a tissue probe into a thin sheath that is adapted to fit any flexible or rigid laryngoscope.

### BACKGROUND OF THE INVENTION

Laryngeal carcinoma is the second most common cancer of the respiratory tract with an overall 5-year survival rate of 60%. Clinical stage at the time of diagnosis and margin status during surgical resection are the two most important factors correlated with disease prognosis. Five-year survival drops from approximately 90% for tumors confined to the larynx, to 60% and 27% for regional and distant disease, respectively. Early diagnosis depends upon careful assessment of suspicious laryngeal lesions noted on diagnostic laryngoscopy. Confirmation requires biopsy and histologic evaluation. Laryngeal tissue sampling is complicated by the need for endoscopic instrument placement in the airway and the heterogeneous appearance of premalignant and malignant lesions. Margin detection during surgical resection of laryngeal carcinoma also requires histologic assessment that is highly dependent on surgeon selected sample sites and the tendency for submucosal tumor extension. Nationally, the incidence of positive margins following endoscopic resection of laryngeal carcinoma is 22%.

### SUMMARY OF THE INVENTION

The present invention provides Raman spectroscopy system, components thereof, and methods of using the same.

In embodiments, the invention comprises an endoscopy sheath-probe device. The device can comprise a sheath, a Raman probe system, an endoscope, or a combination thereof. In embodiments, the sheath comprises a Raman probe channel and an endoscope channel.

The endoscope can comprise a rigid endoscope or a flexible endoscope. In embodiments, the flexible endoscope comprises a fiberoptic endoscope.

The Raman probe system can comprise a probe, a laser source, an excitation signal filter, a collection filter, a charge couple device detector, a signal collection system, a housing unit, a computer, a display, or a combination thereof. In certain embodiments, the signal collection system comprises a spectrum collection range of about 200 cm⁻¹ to about 4000 cm⁻¹. The laser source can comprise a wavelength of about 532 nm, about 638 nm, about 785 nm, or about 1064 nm. In embodiments, the computer is configured for signal analysis and display capabilities.

In certain embodiments, the probe comprises a flexible, fiberoptic probe. The probe can be configured to control an incidence laser angle. In one embodiment, probe comprises an offset distal tip, wherein the offset distal tip is configured to allow contact with a tissue site and control of an optimal incident laser distance. The probe can be configured to control ambient lighting. In embodiments, the excitation signal filter comprises a high-optical density (OD) band-pass filter. The collection filter can comprise a high-optical density long-pass filter. In embodiments, the filter is configured to filter out elastic scattering. In embodiments, the probe comprises an outer diameter of up to about 2.5 mm, a length of up to about 2 m, or a combination thereof.

The probe can be configured to permit laser light delivery and signal collection in real time during an endoscopic procedure. In certain embodiments, the endoscopic procedure comprises an upper airway endoscopic procedure. The upper airway endoscopic procedure can comprise a laryngoscopy.

In embodiments, the endoscope comprises an endoscopic light source and the Raman probe system housing unit further comprises a switch, wherein the switch is configured to permit a user to toggle between the endoscopic light source and the laser source. In certain embodiments, the endoscope comprises an arthroscope, a bronchoscope, a colonoscope, a hysteroscope, a laparoscope, a laryngoscope, a mediastinoscope, sigmoidoscope, thoracoscope, ureteroscope, or an endoscope for use in operative endoscopy. In one embodiment, the endoscopy comprises laryngoscopy. The operative endoscopy can comprise pancreatic laparoscopy.

In one embodiment, the sheath is disposable.

The device can be configured to permit collection of data in real time during an endoscopic procedure. In embodiments, the device is configured to produce a maximum energy exposure of a target tissue, wherein the maximum energy exposure is sufficient to permit collection of Raman data without damaging the tissue. In embodiments, the maximum energy exposure is less than about 5 joules/cm².

In another aspect, the present invention is directed to a method of optical biopsy, IN embodiments, the method comprises the use of any one or more of the devices disclosed herein in a subject. The method can further comprise: imaging a target tissue with the endoscope; subjecting the target tissue to Raman spectroscopy using the Raman probe system to generate Raman spectral data; analyzing the Raman spectral data; and classifying the target tissue as cancer or non-cancer based up on a peak at any one or more wavenumbers from the Raman spectral data. The method can further comprise obtaining a dot product of each normalized Raman signal to generate a 2D Raman image. In embodiments, the target tissue comprises pancreatic tissue, brain tissue, lung tissue, oral tissue, or laryngeal tissue. The cancer can comprise pancreatic cancer, laryngeal cancer, or oral cancer. Non-cancer can comprise non-cancer pancreatic tissue or non-cancer laryngeal tissue.

Another aspect of the present invention includes a computer-aided method of diagnostic assessment of a tissue. In embodiments, the method comprises receiving Raman spectral input data of the tissue; subjecting the Raman spectral input data to at least one data analysis model, wherein the at least one data analysis model uses the Raman spectral input data to classify the tissue as cancer tissue or non-cancer tissue. The data analysis model can comprise principal component (PCA) analysis. The data analysis model can comprises random forest (RF) analysis. In one embodiment, the data analysis model comprises convolutional neural network (CNN) methods.

The Raman spectral input data can comprise 1D Raman spectra or 2D Raman spectra. In certain embodiments, the Raman spectral input data comprises a mean Raman spectrum. The tissue classification can comprise pancreatic cancer tissue or non-cancer pancreatic tissue. In one embodiment, the tissue classification comprises laryngeal cancer tissue or non-cancer laryngeal tissue.

An additional aspect of the present invention comprises a method of diagnosing cancerous tissues. In embodiments, the method comprises positioning patent for the according to a standard of care for a procedure; deploying the device according to any one or more of the various device embodiments disclosed herein, wherein deploying the device comprises inserting the device into the patient; identifying an anatomical tissue of interest; subjecting the anatomical tissue of interest to Raman spectroscopy; and classifying the anatomical tissue as cancerous or non-cancerous.

In embodiments, the sheath comprises one or more channels, such as a Raman probe channel and/or a Laryngoscope channel. “Channel” can refer to a lumen running all or part of the length of the sheath, for example, to support a functional element.

In embodiments, the sheath is a component of a Raman spectroscopy system. For example, the Raman spectroscopy system comprises the system of FIG. 3.

Aspects of the invention are drawn to a Raman spectroscopy system. For example, the Raman spectroscopy system as displayed in FIG. 3.

Aspects of the invention are further drawn to methods of using the Raman spectroscopy system as described herein. For example, a method of identifying a lesion as premalignant or malignant comprising using the Raman spectroscopy system as described herein. For example, the lesion comprises a vocal fold lesion, a laryngeal lesion, a pancreatic cancer, or an oral cancer.

Other objects and advantages of this invention will become readily apparent from the ensuing description.

### BRIEF DESCRIPTION OF THE FIGURES

FIG. 1 shows flexible (upper) and rigid (lower) (O.D.) laryngoscopes. Insets show the distal ends, which can provide illumination.

FIG. 2A shows Raman spectroscopy system for laryngoscopy procedure under one embodiment.

FIG. 2B shows an exemplary Raman spectroscopy system for use in laparoscopic pancreatic cancer surgery.

FIG. 3 shows sheath-probe design containing a Raman spectroscopy system and an endoscope under one embodiment.

FIG. 4A shows a top perspective view of a Raman probe designed for use in the oral cavity under one embodiment

FIG. 4B shows a schematic side view of the FIG. 4A oral cavity Raman probe.

FIG. 4C provides a top schematic view of the FIG. 4A oral cavity Raman probe.

FIG. 4D shows a front schematic view of the FIG. 4A oral cavity Raman probe.

This view shows the tip of the Raman probe extending from the housing and body of the probe.

FIG. 5 shows a close-up view of a Raman probe tip under one embodiment.

FIG. 6 shows an exemplary sheath for containment of the Raman probe and endoscope under one embodiment.

FIG. 7 shows endoscopic resections

FIG. 8 shows histology cassette with the orientation noted.

FIG. 9 shows histology cassettes labeled with peripheral margins.

FIG. 10 shows spectral analysis of laryngeal carcinoma margins.

FIG. 11A shows top perspective view of a Raman laryngoscopy probe under one embodiment.

FIG. 11B provides a side schematic view of the FIG. 11A Raman probe.

FIG. 11C provides a front schematic view of the FIG. 11A Raman probe looking directly into the probe tip.

FIG. 11D provides a detailed schematic view of the probe tip from the FIG. 11A Raman probe.

FIG. 11E shows a top schematic view of the FIG. 11A Raman probe.

FIG. 11F shows top perspective and schematic view of the FIG. 11A Raman probe.

FIG. 12 shows a non-limiting exemplary embodiment of an experimental setup for the ex-vivo Raman analysis of tissue.

FIG. 13 shows a non-limiting, representative total laryngectomy specimen.

FIG. 14 shows a non-limiting, representative endoscopic resection of laryngeal carcinoma specimen in cassette with margins labeled.

FIG. 15 shows non-limiting exemplary schematic diagrams of RF (panel a) and CNN (panel b) for the classification of laryngeal resection tissues.

FIG. 16 shows a non-limiting exemplary receiver operating characteristic curve for the random forest classification of laryngeal carcinoma resection specimens.

FIG. 17 shows graphs of training and testing accuracy and training loss convergence plots for the convolutional neural network classification of laryngeal carcinoma resection specimens.

FIG. 18 shows a non-limiting example of receiver operating characteristic curve for the convolutional neural network classification of laryngeal carcinoma resection specimens.

FIG. 19 shows PCA analysis of Raman spectra of cancer and non-cancer laryngeal resection tissue. Panel a) Original Raman spectra; Panel b) PC1; Panel c) PC2; Panel d) PC3. Key peaks are labeled.

FIG. 20 shows an exemplary embodiment of data processing of one-dimensional (1D) Raman and two-dimensional (2D) Raman images for the convolutional neural network (CNN) model.

FIG. 21 shows an exemplary model of Schematic of the 1D CNN models used to detect pancreatic cancer in a murine cancer model. The stride size was set to 1 to maintain the same sizes of the feature map in each convolutional (Conv) layer and match the visualized CNN features with the corresponding wavenumbers. In the 2D CNN model, all the convolution filters in the 1D CNN were converted into 2D convolution filters; the stride size was changed to 2. All the other configurations were the same as those in the 1D model (Purple blocks: Conv filters; orange blocks: feature maps).

FIG. 22 shows an exemplary 1D Raman and 2D Raman images to identify pancreatic cancer. (Panel a) Original 1D Raman spectrum of pancreatic cancer and (Panel b) corresponding 2D Raman images obtained from the dot product of the 1D Raman spectrum and its transpose. (Panel c) Comparison of original 1D Raman spectrum and the normalized 2D Raman principal component 1 (PC1). (Panel d) Difference between the original 1D Raman spectrum and the normalized 2D Raman PC1.

FIG. 23 shows (Panel a) Accuracy-epoch and (Panel b) loss-epoch curves of 1D Raman, 2D Raman images, and 2D Raman PC1 during CNN training (each curve is the average of five-fold cross-validation, and the learning rate is set to 0.001).

FIG. 24 shows exemplary receiver operating characteristic curves of (Panel a) 1D Raman model, (Panel b) 2D Raman image model, (Panel c) 2D Raman PC1 model, (Panel d) graphs of area under the curve (AUC) for 1D, 2D, and 2D Raman PC1.

FIG. 25 shows non-limiting examples of graphs of (panel a) accuracy, (panel b) sensitivity, and (panel c) specificity of 1D and 2D CNN models used for the distinction between cancerous and normal pancreatic tissues.

FIG. 26 shows non-limiting examples CNN features of the mean spectra of cancerous pancreatic tissue (red line: Tum_MXPL_MaxCh) and normal pancreatic tissue (black line: Pan_MXPL_MaxCh) from the max-pooling layer in (panel a) 1D Raman and (panel b) 2D Raman PC1.

FIG. 27 shows non-limiting examples of CNN features from the max-pooling layer in the mean 2D Raman images. (Panel a) 2D images of CNN features of cancerous (Tum) and normal pancreatic (Pan) tissues from max-pooling layer. (Panel b) Normalized diagonal pixel curves from 2D images of CNN features.

FIG. 28 shows a non-limiting example of Mean CNN feature curves of the test dataset from the max channels of the max-pooling layers (MXPL_MaxCh) in the wavenumber range of 600-2000 cm⁻¹. (Panel a) 1D Raman and (Panel b) 2D Raman PC1 (Tum: pancreatic cancer, Pan: normal pancreas).

FIG. 29 shows Peak frequency at specific wavenumbers occurring in the CNN features of the test dataset from the max channels of the max-pooling layers (MXPL_MaxCh). (Panel a) 1D Raman and (Panel b) 2D Raman PC1. Peak intensity >0.5, such as in FIG. 26.

FIG. 30 shows a non-limiting exemplary graph of Accuracies of individual subregions of 1D Raman and 2D Raman PC1 in classifying cancerous and normal pancreatic tissues.

FIG. 31 shows non-limiting examples of accuracy of (Panel a) 1D Raman and (Panel b) 2D Raman PC1 in classifying pancreatic cancer and normal tissues with the forward (from 600 cm⁻¹ to 3970 cm⁻¹) and backward (from 3970 cm⁻¹ to 600 cm⁻¹) data.

FIG. 32 shows non-limiting examples of graphs of mean CNN feature curves of the test dataset from the max-channels of the max-pooling layers (MXPL_MaxCh) in the full wavelength ranges (600-3970 cm⁻¹). (Panel a) 1D Raman; (Panel b) 2D Raman PC1. Tum: pancreatic cancer; Pan: normal pancreas.

FIG. 33 shows top perspective view of a Raman laryngoscopy probe under an embodiment.

FIG. 35A provides a front schematic view of the FIG. 33 Raman probe.

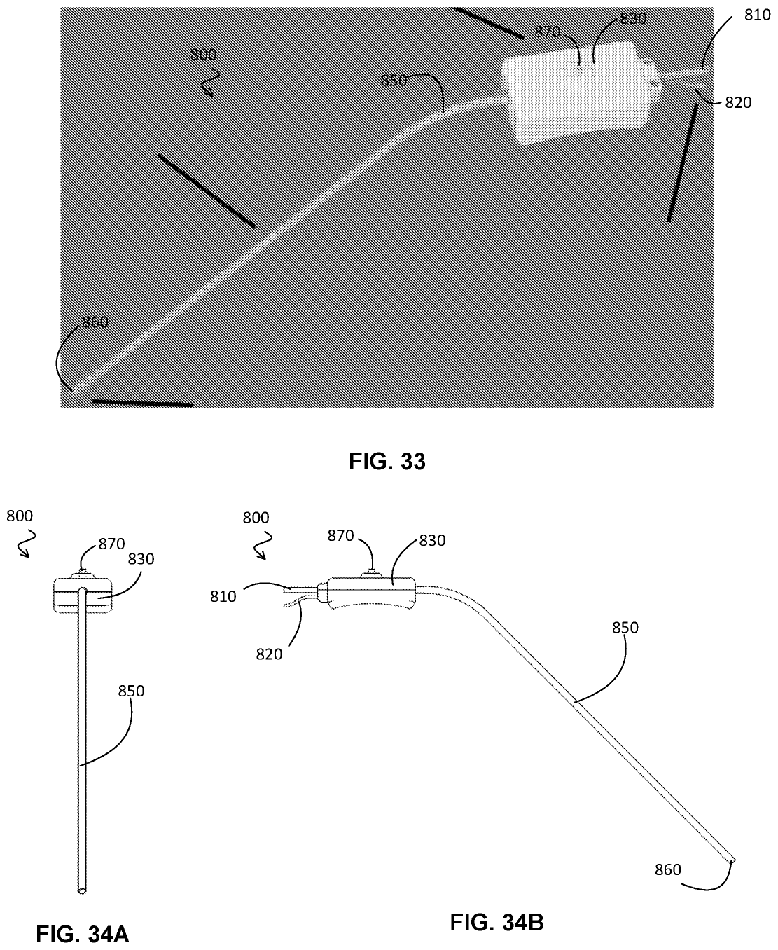

FIG. 34B provides a side schematic view of the FIG. 33 Raman probe.

### DETAILED DESCRIPTION OF THE INVENTION

Detailed descriptions of one or more embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

For purposes of the present disclosure, it is noted that spatially relative terms, such as “up,” “down,” “right,” “left,” “beneath,” “below,” “lower,” “above,” “upper” and the like, can be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features. Thus, the exemplary term “below” can encompass both an orientation of above and below. The device can be otherwise oriented (rotated 90 degrees or at other orientations) and the spatially relative descriptors used herein interpreted accordingly.

As used herein, the term “standard of care” can refer to a diagnostic and/or treatment process for which a clinician follows for a certain type of patient, illness, or clinical circumstance. For example, a standard of care can refer to the ordinary level of skill and care that a clinician is expected to observe in providing clinical care to a patient. In embodiments, the standard of care can vary depending on the patient, the illness, or clinical circumstance. As used herein, “standard care practices” can refer to practices which are standard of care.

As used herein, the term “clinician” can refer to a person qualified in the clinical practice of medicine, psychiatry, or psychology. As used herein, the terms “clinician” and “practitioner” can be used interchangeably. For example, “clinician” can refer to a physician, a surgeon, a veterinarian, a physician assistant, a nurse, or a person practicing under the supervision thereof.

As used herein, the term “board-certified” can refer to a professional whose qualifications have been approved by an official group or governing body. For example, the person is a physician who has graduated from medical school, completed residency, trained under supervision in a specialty, and passed a qualifying exam given by a medical specialty board.

The terms “subject” and “patient” as used herein include all members of the animal kingdom including, but not limited to, mammals, animals (e.g., cats, dogs, horses, swine, etc.) and humans.

The term “cancer surgery” can include a surgical procedure for diagnosis or treatment of a cancer. The term can refer to a procedure for biopsy of potentially cancerous tissue, a procedure for resection or removal of cancerous tissue, or a combination thereof.

Disclosed herein is a sheath-probe device configured such that a Raman spectroscopy system can be deployed during an endoscopic or laparoscopic procedure. In embodiments, the device enables the user to perform an optical biopsy. As used herein, the term “optical biopsy” can refer to the utilization of properties of light to determine a diagnosis of a specimen, target tissue, or tissue of interest. For example, the specimen, target tissue, or tissue of interest can be an organ, a tissue, or a cell. For example, the organ can comprise a heart, an appendix, a larynx, a lymph node, an ovary, a kidney, a liver, a lung, a brain, a pancreas, a bladder, a stomach, an intestinal organ, cheek, gums, a tongue, skin, a tissue thereof, or a cell thereof. As used herein, the term “specimen” can refer to any organ, tissue, cell, or biological sample. As used herein, the terms “specimen,” “sample,” “target tissue,” or “anatomical tissue of interest” can be used interchangeably.

As used herein, “diagnosis” can refer to a characterization or classification of a disease, disorder, or lack thereof from a sign, symptom, or marker. For example, a specimen can be classified as non-malignant, pre-malignant, or malignant. As used herein, the terms “normal”, “non-cancer”, “benign”, and “non-malignant” can be used interchangeably. In embodiments, “pre-malignant” can refer to a state or classification which a specimen has that has a tendency or inclination to become malignant but is not currently classified as malignant based upon conventional standards. For example, the pre-malignant specimen can be keratosis with dysplasia. In embodiments, “malignant” can refer to a state or classification to a specimen has having characteristics of uncontrollable cell growth, infiltration, destruction, metastases, or a combination thereof. For example, a malignant comprises carcinoma. For example, the carcinoma comprises carcinoma in situ or invasive carcinoma. As used herein, the terms “malignant”, “cancer”, and “cancerous” can be used interchangeably.

In embodiments, the device described herein can be used to establish appropriate surgical margins. As used herein, “surgical margins” can refer to the edge of a resected sample. The terms “appropriate” or “adequate” in regard to surgical margins can refer to margins which are not positive for the cells targeted for removal. As used herein, the terms “superficial” in regard to surgical margins can refer to those which are positive. A “positive” margin can refer to a resection that contains the cells targeted for removal on the edge of the resected sample. For example, the cells targeted for removal are cancer cells. In embodiments, positive surgical margins can be indicative of an unsuccessful resection. As used herein, the term “negative” in reference surgical margins can refer to a resection that does not contain cells targeted for removal in the surgical margins. In embodiments, a negative surgical margin can be indicative of a successful resection.

In embodiments, the device described here can be configured to help inform a clinician or practitioner of a disease prognosis or clinical stage. As used herein, a clinician or practitioner can refer to a physician, surgeon, veterinarian, physician assistant, nurse, or student thereof.

In embodiments, the device described herein can be used to grade a specimen or to inform the clinician or practitioner of a grade of a specimen. As used herein, the term “grade” or “grading” can refer to the measure or description of specimen based upon whether the specimen comprises characteristics that are consistent with normal or abnormal specimens. “Grade” can further include a description of how quickly an abnormal specimen is expected to grow or spread.

The device herein can use Raman spectroscopy as a non-destructive tool for biological sample imaging and analysis. As used herein, the term “non-destructive” can refer to the ability to not permanently alter a specimen or the surrounding tissue after being subjected to a condition. When monochromic light from a laser source strikes a sample, the photons are absorbed by its surface and reemitted. The majority of the reemitted light occurs at the same frequency as the monochromic source (such reemitted light can be referred to as “elastic scattering”). If the sample is Raman-active, a portion of the reemitted light will radiate at frequencies above and below the incident frequency (such reemitted light can be referred to as “inelastic scattering”). As used herein, the term “Raman active” can refer to any molecule that exhibits the Raman effect. For example, a molecule that exhibits the Raman effect experiences a change in polarizability as a result of molecular vibration. This inelastic scattering of the photons by the component(s) in the sample can be used to generate a Raman spectrum, from which a structural fingerprint can be identified. As used herein, a “structural fingerprint” can refer to a spectral characteristic that is indicative of a molecular feature or structure. For example, the signal can comprise Raman shift (cm⁻¹). For example, the molecular feature can be indicative of a functional group or other structural characteristic. For example, peaks and frequency of peaks at specific wavenumbers can be related to specific biological components. For example, the biological components are protein contents. For example, the protein content can be collagen. For example, the biological components can comprise lipids, nucleic acids, or a combination thereof. In embodiments, biological components in a specimen can be indicative of a cancerous or non-cancerous specimen.

### DETAILED DESCRIPTION OF SELECTED EMBODIMENTS

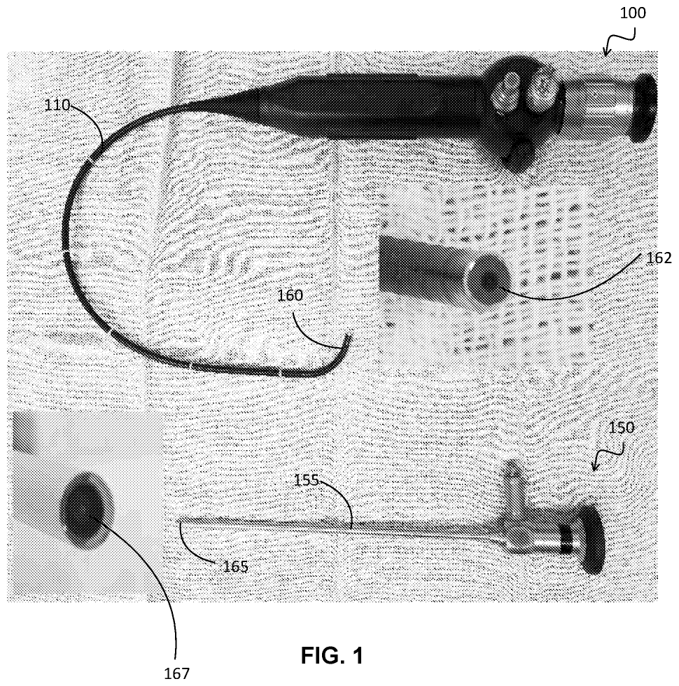

FIG. 1 shows exemplary endoscopes 100, 150 that can be used in various embodiments of the present invention. A flexible endoscope 100 is shown at the top of the figure, and a rigid (O.D.) laryngoscope 150, can be seen at the bottom of the figure. The flexible endoscope 100 comprise a flexible shaft 110, which terminates in a flexible tip 160 at the distal end of the shaft. A rigid laryngoscope 150 comprise a rigid shaft 155, which terminates in a rigid tip 165 at the distal end of the shaft. As seen in the insets, the end of the flexible tip 160 or rigid tip 165 comprises an termination point 162, 167. In embodiments, the termination point 162, 167 can include a light source for illumination of target tissue. The termination point 162, 167 can further comprise a water nozzle, a water jet, and air nozzle, light guide, an objective lens, and instrument channel, such as an channel for a suction device or biopsy device, or a combination thereof.

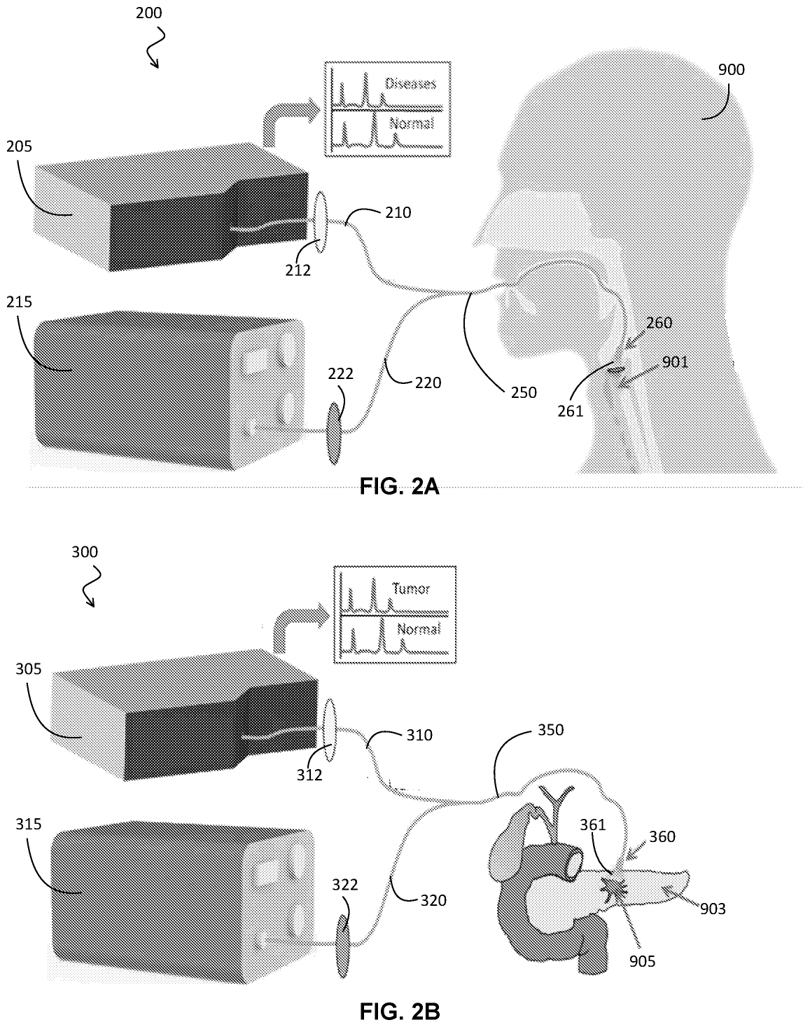

FIG. 2A shows Raman spectroscopy system 200 for a laryngoscopy procedure under one embodiment. In this embodiment, the system 200 comprises a laser source 215 and a Raman spectrometer 205. As can be seen, a laser fiber 220 can be connected to the laser source 215, such that an incident laser (also referred to herein as an “excitation laser”) can be transmitted from the laser source 215 to the surgical site or a tissue of interest. In embodiments, an excitation filter 222 can be placed along the path of the incident laser signal. A collection fiber 210 can be connected to the Raman spectrometer 205 to permit transmission of a collected signal to the spectrometer 205. At least one collection filter 212 can be placed along the path of the collected signal to filter out noise from the collected signal prior to the signal reaching the spectrometer. In embodiments, the laser fiber 220 and the collection fiber 210 can be combined for at least a portion of their length and run parallel with one another to form a single Raman probe shaft 250 such that the laser fiber 220 and the collection fiber 210 can be introduced simultaneously into an orifice or opening of a subject 900 to access a surgical site or tissue target of interest 901. The Raman probe shaft 250 can terminate in a Raman probe tip 260, which permits collection of a Raman signal from a surgical site or a tissue of interest 901. In operation of the FIG. 2A Raman spectroscopy system 200, the Raman probe 250 can be inserted into the mouth or nose of a subject 900 such that the Raman detector is placed on or near a surgical site or a tissue of interest, such as the larynx 901. The tissue of interested 901 is then illuminated with the excitation laser 261, and the resultant scattered radiation is collected via the Raman detector and sent back through the collection fiber 210 and collection system to the Raman spectrometer 205 for signal analysis to determine whether the particular point illuminated in the target tissue 901 is diseased or normal.

FIG. 2B shows an exemplary Raman spectroscopy system 300 for use in laparoscopic pancreatic cancer surgery. In this embodiment, the system 300 comprises a laser source 315 and a Raman spectrometer 305. As can be seen, a laser fiber 320 can be connected to the laser source 315, such that an incident laser (also referred to herein as an “excitation laser”) can be transmitted from the laser source 315 to the surgical site or a tissue of interest. In embodiments, an excitation filter 322 can be placed along the path of the incident laser signal. A collection fiber 310 can be connected to the Raman spectrometer 305 to permit transmission of a collected signal to the spectrometer 305. At least one collection filter 312 can be placed along the path of the collected signal to filter out noise from the collected signal prior to the signal reaching the spectrometer. In embodiments, the laser fiber 320 and the collection fiber 310 can be combined for at least a portion of their length and run parallel with one another to form a single Raman probe shaft 350 such that the laser fiber 320 and the collection fiber 310 can be introduced simultaneously into an orifice or opening of a subject to access a surgical site or tissue target of interest 903. The Raman probe shaft 350 can terminate in a Raman detector 360, which permits collection of a Raman signal from a surgical site or a tissue of interest 905. In operation of the FIG. 2B Raman spectroscopy system 300, the Raman probe 350 can be inserted into a surgical opening of a subject such that the Raman detector 360 can be placed on or near a surgical site or a tissue of interest 905, such as the pancreas 903. The tissue of point of interest 903 is then illuminated with the excitation laser 361, and the resultant scattered radiation is collected via the Raman probe tip 360 and sent back through the collection fiber 310 and collection system to the Raman spectrometer 305 for signal analysis to determine whether the particular point illuminated 905 in the target tissue 903 is diseased or normal. In certain embodiments, the clinician will determine if the particular point illuminated 905 on the target tissue 903 is a cancerous tumor or noncancerous.

In embodiments, the Raman probe comprises a hand-held probe configured for use during a medical procedure.

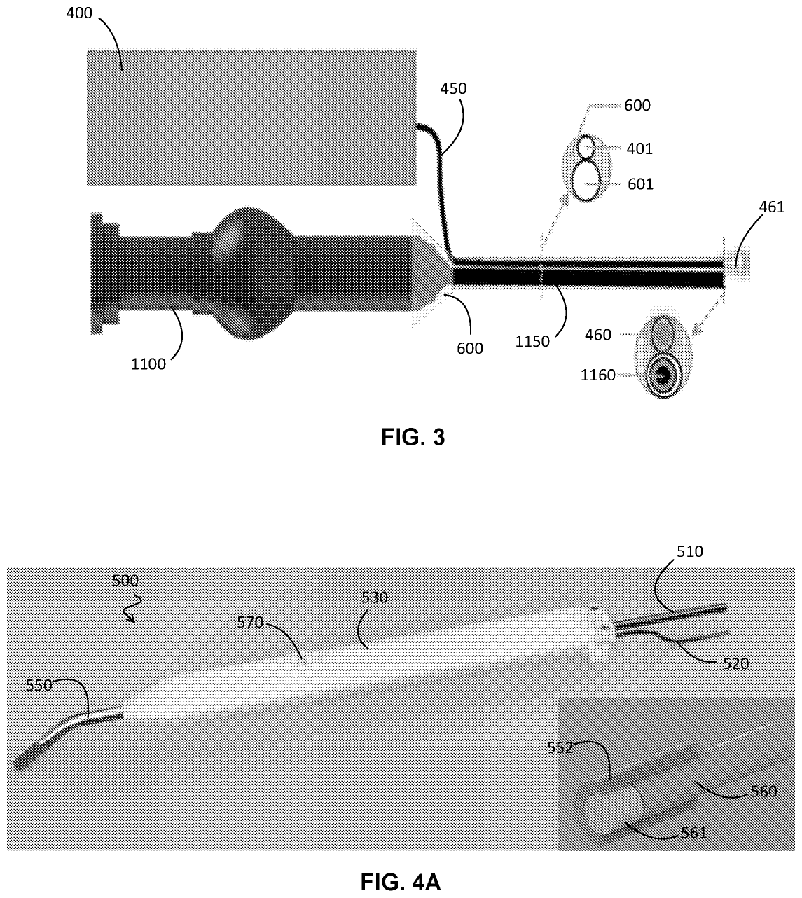

In embodiments, the Raman spectroscopy systems disclosed herein can be used in conjunction with an endoscope, such as those disclosed in FIG. 1, or laparoscope during a medical procedure. As shown in the exemplary embodiment of FIG. 3, a sheath-probe design can be employed when a Raman system 400 is utilized in connection with an endoscope 1100 or laparoscope. In embodiments a sheath 600 can be employed wherein the sheath 600 is configured to hold both the Raman probe shaft 450 and the shaft 1150 of an endoscope. A cross-section of the sheath 600 reveals a Raman probe channel 401 and an endoscope channel 601, wherein the Raman probe channel 401 is configured to hold a given length of the Raman probe shaft 450, and the endoscope channel 601 is configured to hold a given length of the endoscope or laparoscope shaft 1150. A view looking directly into the distal end of the sheath-probe design reveals the distal tip 460 of the Raman probe shaft 450 and the distal tip 1160 of the endoscope or laparoscope. In embodiments, the sheath 600 can comprise at least two channels 401, 601. The sheath 600 can comprise up to five sheath channels. In certain embodiments, the sheath 600 comprises one, two, three, four, five, six, seven, eight, nine, or ten channels. The sheath channels can comprise a lumen, hole, gap, notch, void, or passageway configured to hold the shaft of a medical device or an associated accessory. In embodiments, the sheath channels are configured to hold a Raman probe as disclosed herein, an endoscope shaft, a laparoscope shaft, the shaft of an irrigation instrument, the shaft of a suction instrument, the shaft of a biopsy instrument, the shaft of an air line, or a combination thereof.

In embodiments the sheath 600, the Raman probe shaft 450, or both are reusable. The sheath 600, the Raman probe shaft 450, or both can be sterilizable. In certain embodiments, the sheath 600, the Raman probe shaft 450, or both are disposable.

In reusable embodiments, the sheath 600, the Raman probe shaft 450, or both can be sterilized or can be sterile when provided or obtained. By way the example, the sheath 600, the Raman probe shaft 450, or both can be configured to withstand autoclave sterilization, chemical sterilization, x-ray sterilization, or a combination thereof. In alternate embodiments, the sheath 600, the Raman probe shaft 450, or both can be configured for a single use.

The sheath 600, the Raman probe shaft 450, or both can be comprised of any material currently known by those of skill in the art or later developed that is suitable for use in medical or surgical procedures. In embodiments, the sheath 600, the Raman probe shaft 450, or both are comprised of a medical-grade material. The sheath 600, the Raman probe shaft 450, or both can be comprised of a medical grade polymer, metal, or a combination thereof. In certain embodiments, the sheath 600, the Raman probe shaft 450, or both are comprised of surgical metal. The sheath 600, the Raman probe shaft 450, or both can be comprised of stainless steel, titanium, tantalum, gold, platinum, palladium, or any other metal or combination of metals suitable for surgical use.

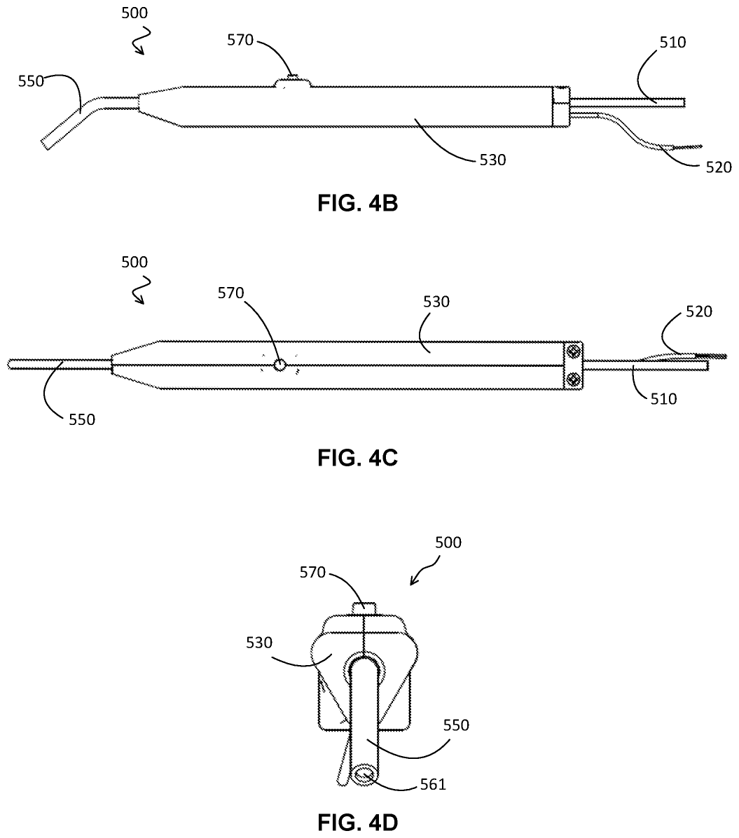

FIG. 4A shows a top perspective view of a Raman probe 500 designed for use in the oral cavity under one embodiment. In this embodiment, the Raman probe 500 comprises a Raman probe shaft 550, a housing 530, and a switch 570. In certain embodiments, the Raman probe 550 can comprise a collection fiber 510 and a laser fiber 520, which are combined and contained within the Raman probe shaft 550.

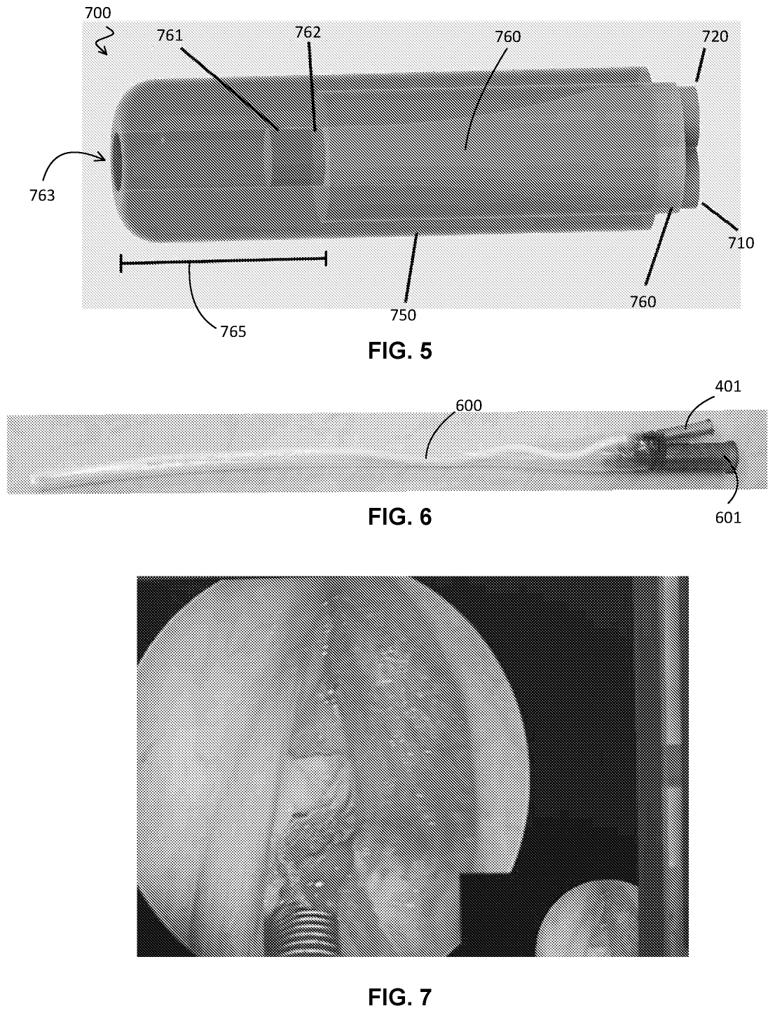

The inset of FIG. 4A provides a close-up view of a Raman probe tip under one embodiment. As can be seen, a portion of the Raman probe shaft 552 is removed to reveal the Raman probe fiber 560 enclosed therein. In this embodiment, the Raman probe fiber 560 does not extend to the distal-most point of the Raman probe shaft 552 such that there is an offset between the tip of the Raman probe fiber 560 and the end of the Raman probe shaft 552. In embodiments, this tip offset is calculated to provide the optimal distance between the incident laser and the specimen or tissue of interest. For instance, in operation, the distal-most portion of the Raman shaft 552 can be placed against the tissue of interest, and the distance tip offset can provide the optimal distance for obtaining reliable Raman spectral data. The tip offset can be further configured to prevent inadvertent collection of ambient light such that only resultant reemission from the excitation laser is collected via the collection fiber and transmitted through the Raman signal collection system. As can be seen in this embodiment a lens 561 can also be disposed within the Raman probe shaft 552, wherein the lens 561 comprises a termination point that is flush with that of the Raman probe shaft 552 such that there is no gap or empty space created between at the end of Raman probe shaft 552 (as contrasted to the embodiment of FIG. 5). Such a configuration can be configured to prevent the build-up of contaminants within tip offset of the Raman probe shaft 552 and permit simplified sterilization of the Raman probe 500 following use.

FIG. 4B shows a schematic side view of the FIG. 4A oral cavity Raman probe. In this view, the Raman probe shaft 550 can be seen extending from one end of the probe 500, and angled downward for ease of contact with a target tissue when in use. The Raman probe laser fiber 520 and collection fiber 510 can be seen exiting the back portion of the probe 500. As discussed further herein, certain embodiments can comprise control cables exiting the Raman probe, which can be utilized to enable mechanical manipulation of the probe 500 or control other probe parameters.

FIG. 4C provides a top schematic view of the FIG. 4A oral cavity Raman probe 500.

FIG. 4D shows a front schematic view of the FIG. 4A oral cavity Raman probe 500. This view shows the opening of the Raman probe shaft 550 in the foreground to reveal the tip of a lens 561 residing therein. The housing 530 can be seen in the foreground surrounding the Raman probe shaft 550 with a switch 570 positioned on the top of the probe 500.

FIG. 5 shows a close-up view of a Raman probe 700 near the distal end under another embodiment. The Raman probe shaft 750 is shown in phantom to reveal various exemplary Raman components residing therein. As can be seen, the laser fiber 720 and the collection fiber 710 can be joined together and enclosed within a single Raman fiber 760 which can be further enclosed within the Raman probe shaft 750. In this particular embodiment, the Raman fiber 760 does not extend to the distal-most portion of the Raman probe shaft 750 such that there exists a fiber tip offset 765 between the tip of the Raman fiber 760 and the end of the Raman probe shaft 750. A dichroic filter 762 can be seen within the Raman probe shaft 750 just distal to the termination point of the Raman fiber 760. In addition, a lens 761 is shown distal to the dichroic filter 762. In this embodiment, the lens does not extend to the distal-most point of the Raman fiber shaft 750, such that an empty space or gap exists within the fiber tip offset 765 at the distal-most portion of the Raman fiber shaft 750. In alternate embodiments, the lens completely fills the space between the dichroic filter 762 and the distal-most portion of the Raman fiber shaft 750 (e.g. see the inset of FIG. 4A) such that there is no empty space within the fiber tip offset 765.

FIG. 6 shows an exemplary sheath for containment of the Raman probe shaft 401 and endoscope shaft 601 under one embodiment.

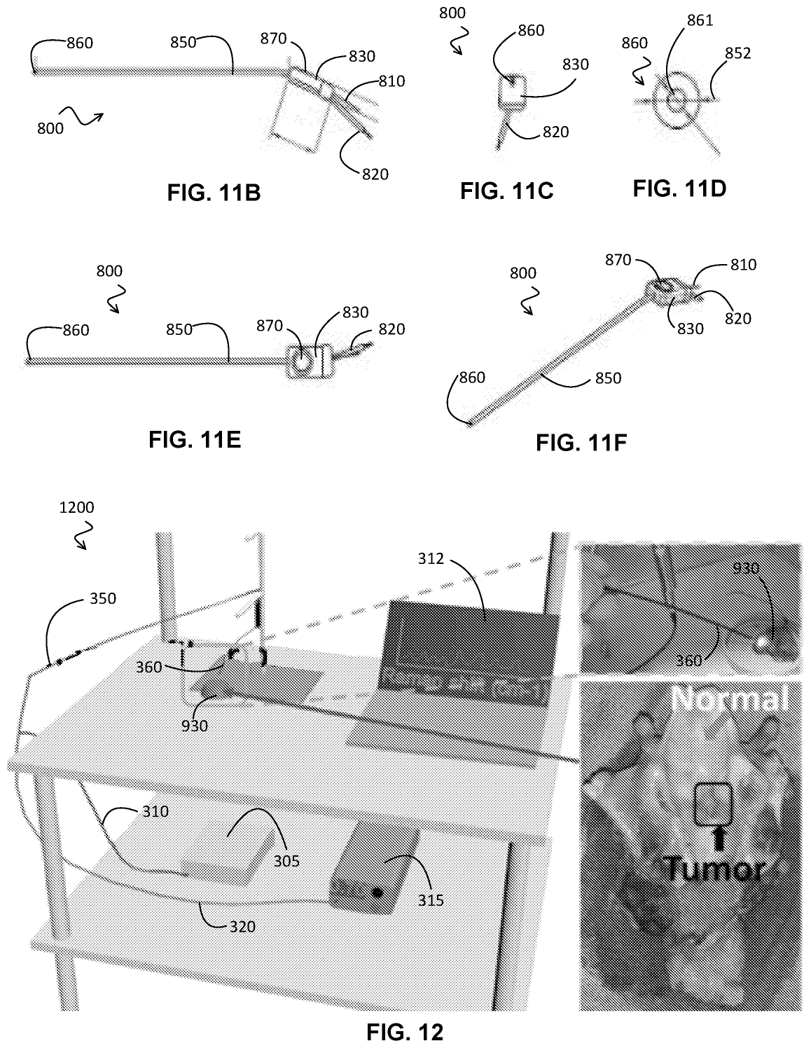

FIG. 11A shows top perspective view of a Raman probe 800 for use with laryngoscopy under one embodiment of the present invention. As can be seen, the probe 800 comprise a housing 830 with a switch 870 residing thereon. Further, the Raman probe shaft 850 can be seen extending at a downward angle form the housing 830 until it terminates at the Raman probe tip 860, which can contain a fiber tip offset (as more clearly seen in the inset of FIG. 4A and at 765 of FIG. 5).

FIG. 11B provides a side schematic view of the FIG. 11A Raman probe 800.

FIG. 11C provides a front schematic view of the FIG. 11A Raman probe 800 looking directly into the probe tip 860.

FIG. 11D provides a detailed schematic view of the probe tip 860 from the FIG. 11A Raman probe 800.

FIG. 11E shows a top schematic view of the FIG. 11A Raman probe 800. The switch 870 can be clearly seen on top of the housing 830.

FIG. 11F shows top perspective and schematic view of the FIG. 11A Raman probe 800.

FIG. 12 shows a non-limiting exemplary embodiment 1200 of an experimental setup for the ex-vivo Raman analysis of tissue 930. In this embodiment, the system 1200 comprises a laser source 315 and a Raman spectrometer 305. As can be seen, a laser fiber 320 can connect to the laser source 315, such that an incident laser can be transmitted from the laser source 315 to the specimen 930. A collection fiber 310 can be connected to the Raman spectrometer 305 to permit transmission of a collected signal to the spectrometer 305. In embodiments, the laser fiber 320 and the collection fiber 310 can be combined for at least a portion of their length and run parallel with one another to form a single Raman probe fiber 350. The Raman probe shaft 350 can terminate in a Raman probe tip 360, which permits collection of a Raman signal from the specimen 930. The system 1200 can further comprise a computer 312 with a display for displaying analytical results from the Raman spectrometer 305. In operation of the FIG. 12 Raman spectroscopy system 1200, the specimen 930 can be placed at an appropriate location and orientation within a collection area of the Raman detector 360. The specimen 930 is then illuminated with the excitation laser (as shown in the top inset), and the resultant scattered radiation is collected via the Raman detector 360 and sent back through the collection fiber 310 and collection system to the Raman spectrometer 305 for signal analysis and display via the computer 312 to determine whether the particular point illuminated in the specimen 930 is diseased or normal. For instance, as shown in the bottom inset of FIG. 12, the Raman data can determine whether the particular point illuminated on the specimen 930 is a tumor or normal.

FIG. 33 shows top perspective view of a Raman probe 800 for use with laryngoscopy under another embodiment. As can be seen, the probe 800 comprise a housing 830 with a switch 870 residing thereon. Further, the Raman probe shaft 850 can be seen extending at a downward angle form the housing 830 until it terminates at the Raman probe tip 860, which can contain a fiber tip offset (as more clearly seen in the inset of FIG. 4 and at 765 of FIG. 5).

FIG. 35A provides a front schematic view of the FIG. 33 Raman probe.

FIG. 34B provides a side schematic view of the FIG. 33 Raman probe.

In certain embodiment, the switch 570, 870 is configured to permit activation or engagement of the excitation laser and signal collection. The switch 570, 870 can be configured to turn off a light source. The switch 570 can be configured to turn off a light source and active the excitation laser. In certain embodiments, the switch 570, 870 is configured to simultaneously turn off the light source and turn on the excitation laser. The Raman probe 500, 800 can comprise control cables configured to control activation of the excitation laser, deactivation of the excitation laser, activation of the light source, deactivation of the light source, activation of the Raman spectrometer for signal collection, positioning of the Raman probe tip, positioning of the incident laser angle, or a combination thereof.

In embodiments, the Raman probe, the sheath 600 or a combination thereof comprise an outer diameter that is sufficient to fit within the nasal cavity or oral cavity of a patient. In embodiments, the outer diameter of the Raman probe, the sheath, or a combination thereof comprises an outer diameter of up to about 20 mm. In embodiments, the outer diameter of the Raman probe, the sheath, or a combination thereof comprises an outer diameter of up to about 10 mm. The outer diameter of the Raman probe, the sheath, or a combination thereof can comprise an outer diameter of about 1 mm, about 2 mm, about 3 mm, about 4, mm, about 5 mm, about 6 mm, about 7 mm, about 8 mm, about 9 mm, or about 10 mm. In certain embodiments, the outer diameter of the Raman probe, the sheath, or a combination thereof comprises a diameter of about 0.25 mm, about 0.5 mm, about 0.75 mm, about 1 mm, about 1.25 mm, about 1.5 mm, about 1.75 mm, about 2 mm, about 2.25 mm, about 2.5 mm, about 2.75 mm, about 3 mm, about 3.25 mm, about 3.5 mm, or about 4 mm.

In certain embodiments, the various embodiments described herein, comprises a sheath 600, a Raman probe system 200, 300, 400, an endoscope 100, 200, 1200, or a combination thereof. The Raman probe system described herein can comprise a Raman probe 250, 350, 450, 500, 800, a laser source 215, 315, an excitation signal filter 222, a collection filter 212, a charge couple device detector, a signal collection system, a housing unit 530, 830, a computer 312, a display, or a combination thereof. In embodiments, the endoscope can comprise an arthroscope, a bronchoscope, a colonoscope, a hysteroscope, a laparoscope, a laryngoscope, a mediastinoscope, sigmoidoscope, thoracoscope, ureteroscope, or an endoscope for use in operative endoscopy. As used herein, the term “endoscopy” can refer to any means for examining inside the body for medical purposes. As used herein, the term “endoscope” can refer to any instrument used to examine the interior of a body.

In embodiments, the endoscope comprises a rigid endoscope 150 or a flexible endoscope 110. For example, the flexible endoscope comprises a fiberoptic endoscope. In embodiments, the Raman probe comprises a flexible endoscope. In further embodiments, the Raman probe is a fiberoptic Raman probe. As used herein, the term “fiberoptic” can refer to the medium or technology with transmission of information as light pulses along a glass or plastic strand or fiber. In embodiments, the fiberoptic laryngoscope can be used in nasolaryngoscopy.

In embodiments, the laryngoscopy can comprise direct laryngoscopy and indirect laryngoscopy. As used herein, the term “direct laryngoscopy” can refer to a laryngoscopy wherein the clinician can visualize the anatomical area of interest by direct line of sight. As used herein, the term “indirect laryngoscopy” can refer to a laryngoscopy wherein the clinician visualizes the anatomical area of interest by means other than direct line of sight. For example, indirect laryngoscopy can comprise mirror or video-based visualizations. In certain embodiments, a camera can be utilized to obtain images of the anatomical area, which are transmitted the clinician for review. In embodiments, a flexible endoscope is used for indirect laryngoscopy.

As described herein, the term “sheath” can refer to a structure that envelopes the other. In embodiments, the device described herein comprises a sheath which envelopes a Raman probe shaft, an endoscopic probe shaft, or a combination thereof. The sheath dimensions of the device described herein can be varied to accommodate various flexible and rigid endoscopes. In embodiments, the Raman probe and endoscopic probe are deployed simultaneously via the sheath. In embodiments, the Raman probe can be deployed prior to the endoscopic probe. In embodiments, the endoscopic probe can be deployed prior to the Raman probe.

The device described herein can be configured to reduce or eliminate ambient or interfering light. In embodiments, the device can electronically turn off the endoscopic light during Raman spectrum acquisition. For example, an electronic control system can engage the Raman analysis while simultaneously switching off the endoscopic illumination. In embodiments, an electronic switch 570, 870 connects the probe fiber to the Raman spectrometer and a light emitting diode (LED) aiming beam. The housing unit 530, 830 can comprise a switch 570, 870 and circuitry to activate the Raman spectrometer and toggle between the LED source (e.g. the endoscopic light) and the incident laser signal. In certain embodiments, the probe instrument tip design can prevent introduction of ambient light due to a fiber tip offset (see the inset of FIG. 4A and 765 of FIG. 5) when the Raman probe shaft 552, 750 contacts with the tissue site of interest.

The probe instrument tip design can further be configured to control of the incident laser angle. In certain embodiments, the clinician can control the incident laser angle by hand, such as via rotation of the Raman probe until the desired incident laser angle is achieved.

In various embodiments, the presently disclosed device is configured to allow for signal detection in real-time. As used herein, the phrase “real time” indicates that data detection and collection becomes available to internal or external users as soon as the data is available or generated or within seconds or milliseconds of such data availability or generation. In certain embodiments, tissue analysis (e.g. cancerous versus non cancer, diseased versus normal, etc.) is transmitted in “real time.” In embodiments, “real time” can mean that less than about one minute. “Real time” can be less than about one second. In embodiments, “real time” can be less than about 100 milliseconds. “Real time” can be about 100 milliseconds, about 200 milliseconds, about 300 milliseconds, about 400 milliseconds, about 500 milliseconds, about 600 milliseconds, about 800 milliseconds, or about 900 milliseconds.

In embodiments, the laser source of the Raman system comprises a diode laser. The diode laser can comprise wavelengths in the visible, near infrared, infrared, near ultraviolet, or ultraviolet spectrum. In embodiments, the diode laser excitation wavelengths comprise about 532 nm, about 638 nm, about 785 nm, about 1064 nm.

In embodiments, the “optimal distance” can refer to the distance between the distal end of the Raman laser fiber and the specimen. The term “incident laser angle” can refer to the angle at which the incident laser (also referred to herein as “excitation laser” contacts the specimen or the angle between the sample and the Raman probe. In certain embodiments, the optimal distance, the incident laser angle, or both can depend upon the beam size. The optimal distance can vary with the incident angle. The incident angle can vary with the optimal distance. In embodiments, the optimal laser distance comprises about 0 mm to about 20 mm. the optimal distance can be about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, or about 15 mm. In certain embodiments, the optimal distance is greater than about 20 mm. The optimal distance can be less than about 1 mm.

In embodiments, the systems, devices, and methods disclosed herein are configured to permit collection of reliable Raman data without damaging the target tissue. Embodiments are designed to enable tissue laser exposure times of less than about 30 seconds, less than about 20 seconds, less than about 10 seconds, less than about 5 seconds, less than about 2.5 seconds, or less than about 1 second. In embodiments, the maximum energy exposure of tissue subjected to the incident laser is less than that required to cause histological damage to skin. The maximum energy exposure can be less than that required to reveal histological damage to a mucosal layer. In one embodiment, the maximum energy exposure of the tissue of interest is less than about 6 joules, less than about 5 joules, less than about 4 joules, less than about 3 joules, less than about 2 joules, or less than about 1 joule. In one embodiment, the maximum energy exposure is less than about 5 joules/cm², less than about 4 joules/cm², less than about 3 joules/cm², less that about 2 joules/cm², or less than about 1 joule/cm², In an embodiment, the maximum energy exposure to tissue subjected to the incident laser is about 2.2 joules/cm², about 2.18 joules/cm², about 2.16 joules/cm², about 2.14 joules/cm², about 2.12 joules/cm², about 2.10 joules/cm², about 2.08 joules/cm², about 2.06 joules/cm², about 2.04 joules/cm², about 2.02 joules/cm², about 2.0 joules/cm², about 1.8 joules/cm², about 1.6 joules/cm². about 1.4 joules/cm², about 1.2 joules/cm², about 1.0 joules/cm², about 0.8 joules/cm², about 0.6 joules/cm², about 0.4 joules/cm², or about 0.2 joules/cm².

Embodiments of the device comprise machine learning assisted analysis of Raman signals to differentiate between normal, pre-malignant, and malignant tissue samples. As used herein, the phrase “machine learning” can refer artificial intelligence or software algorithms used to implement supervised learning, unsupervised learning, reinforcement learning, or any combination thereof. For example, the algorithm can learn from and make predictions about data. As used herein, the phrase “deep learning” can refer to a form of machine learning that utilizes multiple interconnected neural network layers along with feedback mechanisms or other methods to improve the performance of the underlying neural network.

Spectral analysis methods described herein comprise principal component analysis (PCA), random forest (RF), and one-dimensional convolutional neural network (CNN) methods.

In embodiments, the devices and methods disclosed herein can be communicatively coupled with computer networks, computing devices, mobile devices, or combinations thereof. Under certain embodiments, the systems and methods disclosed herein can utilize the communicative coupling to relay data collected from the collection system. Such data can include, for example, the Raman spectral data.

The communicative coupling can be accomplished through one or more wireless communications protocols. The communicative coupling can comprise a wireless local area network (WLAN). A WLAN connection can implement WiFi™ communications protocols. Alternatively, the communicative coupling can comprises a wireless personal area network WPAN. A WPAN connection can implement Bluetooth™ communications protocols.

Embodiments can comprise a data port for relaying data to the mobile device or other computing device. The data port can comprise a USB connection or any other type of data port. The data port allows for a wired communication between the collection system and separate computing devices. The data port can be used alone or in combination with the wireless communications protocols of the systems and device described above.

Computer networks suitable for use with the embodiments described herein include local area networks (LAN), wide area networks (WAN), Internet, or other connection services and network variations such as the world wide web, the public internet, a private internet, a private computer network, a public network, a mobile network, a cellular network, a value-added network, and the like. Computing devices coupled or connected to the network can be any microprocessor controlled device that permits access to the network, including terminal devices, such as personal computers, workstations, servers, mini computers, main-frame computers, laptop computers, mobile computers, palm top computers, hand held computers, mobile phones, TV set-top boxes, or combinations thereof. The computer network can include one of more LANs, WANs, Internets, and computers. The computers can serve as servers, clients, or a combination thereof.

One or more components of the systems and methods described herein and/or a corresponding interface, system or application to which the systems and methods described herein are coupled or connected includes and/or runs under and/or in association with a processing system. The processing system includes any collection of processor-based devices or computing devices operating together, or components of processing systems or devices, as is known in the art. For example, the processing system can include one or more of a portable computer(s), portable communication device operating in a communication network, a network server, or a combination thereof. The portable computer can be any of a number and/or combination of devices selected from among personal computers, personal digital assistants, portable computing devices, and portable communication devices, but is not so limited. The processing system can include components within a larger computer system.

The processing system of an embodiment includes at least one processor. The term “processor” as generally used herein refers to any logic processing unit, such as one or more central processing units (CPUs), digital signal processors (DSPs), application-specific integrated circuits (ASIC), etc. The processor can be disposed within or upon a single chip.

The processing system can further include at least one memory device or subsystem. The processing system can also include or be coupled to at least one database. The processor and memory can be monolithically integrated onto a single chip, distributed among a number of chips or components, and/or provided by some combination of algorithms. The systems and methods described herein can be implemented in one or more of software algorithm(s), programs, firmware, hardware, components, circuitry, in any combination.

The components of any system that include the systems and methods described herein can be located together or in separate locations. Communication paths couple the components and include any medium for communicating or transferring files among the components. The communication paths include wireless connections, wired connections, and hybrid wireless/wired connections. The communication paths also include couplings or connections to networks including local area networks (LANs), metropolitan area networks (MANs), wide area networks (WANs), wireless personal area networks (WPANs), proprietary networks, interoffice or backend networks, and the Internet. Furthermore, the communication paths include removable fixed mediums like floppy disks, hard disk drives, and CD-ROM disks, as well as flash RAM, Universal Serial Bus (USB) connections, RS-232 connections, telephone lines, buses, and electronic mail messages.

Aspects of the systems and methods described herein can be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the systems and methods described herein include: microcontrollers with memory (such as electronically erasable programmable read only memory (EEPROM)) or without memory, embedded microprocessors, firmware, software, etc. Furthermore, aspects of the systems and methods of surgical simulation described herein can be embodied in microprocessors having software-based circuit emulation, discrete logic (sequential and combinatorial), custom devices, fuzzy (neural) logic, quantum devices, and hybrids of any of the above device types. Of course the underlying device technologies can be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

It is noted that any system, method, and/or other components disclosed herein can be described using computer aided design tools and expressed (or represented), as data and/or instructions embodied in various computer-readable media, in terms of their behavioral, register transfer, logic component, transistor, layout geometries, and/or other characteristics. Computer-readable media in which such formatted data and/or instructions can be embodied include, but are not limited to, non-volatile storage media in various forms (e.g., optical, magnetic or semiconductor storage media) and carrier waves that can be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, etc.). When received within a computer system via one or more computer-readable media, such data and/or instruction-based expressions of the above described components can be processed by a processing entity (e.g., one or more processors) within the computer system in conjunction with execution of one or more other computer programs.

### Non-Limiting Exemplary Embodiments for Clinical Use

Described herein are non-limiting, exemplary embodiments for clinical use of the device(s) described herein. As standard of care is determined by the type of patient, illness, and clinical situation, the embodiments described herein are non-limiting and exemplary in nature and can be adapted for standard of care practice by one of ordinary skill in the art. For example, one of ordinary skill comprises a board-certified physician, board certified surgeon, or a person practicing under the supervision thereof.

POSITIONING THE PATIENT—The patient can be positioned according to standard of care. For example, if a patient is to be subjected to direct laryngoscopy, the patient can be placed in a “sniffing” position. For example, the sniffing position can comprise placing the patient in a supine position, elevating the head about 15°, and extending the neck about 35°. In embodiments, the patient's mouth can then be opened by flexing the thumb and middle finger past each other such that the thumb is pressing on the mandibular dentition and the middle finger is pressing on the maxillary dentition.

For example, if the patient is to be subjected to an upper GI endoscopy procedure, the patient can be placed in a left lateral decubitus position with the chin tucked against the chest. A bite guard can then be placed between the patient's teeth or fitted over the device sheath.

For example, if the patient is to be subjected to a lower GI endoscopy, the patient can be placed in a left lateral decubitus position.

For example, if the patient is to be subjected to a laparoscopic procedure, the patient can be placed in a supine position.

DEPLOYING THE DEVICE—The device can be deployed according to the standard of care for deploying an endoscopic or laparoscopic procedure. For example, if a patient is to be subjected to direct laryngoscopy, the laryngoscope can be inserted in the right side of the mouth and the Raman probe can be deployed similarly to that of a suction catheter. In embodiments, the Raman probe shaft and the laryngoscope can be deployed simultaneous via a sheath-based delivery system as more particularly described herein (see, for example FIG. 3). In certain embodiments, the Raman probe shaft can occupy one channel of the sheath and a suction catheter can occupy a second channel of the sheath.

For example, if a patient is to be subjected to an upper GI endoscopy, and a bite guard has been placed in the patient's mouth, the presently disclosed device can be slid into the patient's mouth through the bite guard. If the bite guard has been fitted over the presently disclosed device sheath rather than directly in the patient's mouth, the bite guard will not be placed until the presently disclosed device has entered the upper esophagus of the patient.

For example, if a patient is to be subjected to a lower GI endoscopy, the clinician can lubricate the device for insertion into the anus and advance the device to the desired location.

For example, if a patient is to be subjected to a laparoscopic procedure, the clinician can make an incision in the patient's skin to create a port. For example, the incision can be made on the abdomen. For example, the incision size can be about 0.5 to about 1.0 cm in diameter. The incision size can be less than about 0.5 cm. In embodiments, the incisions size is greater than about 1.0 cm. The incision size can be about 0. 1 cm, about 0.2 cm, about 0.3 cm, about 0.4 cm, about 0.5 cm, about 0.6 cm, about 0.7 cm, about 0.8 cm, about 0.9 cm, about 1 cm, about 1.1 cm, about 1.2 cm, about 1.3 cm, about 1.4 cm, or about 1.5 cm. In embodiments, a trochar can be inserted into the port and the device can be deployed via the trochar.

IDENTIFYING ANATOMICAL TISSUE—The identification of anatomical tissue of interest and surgical margins can be determined according to standard of care for a particular anatomical tissue and clinical scenario. For example, identification of anatomical tissue can be performed through visualization with the naked eye, a microscope, an endoscope, a laparoscope, a camera/video device, or a mirror. In embodiments, one of ordinary skill in the art (e.g., a surgeon) can visualize a tissue identify anatomical tissue as a tissue of interest. For example, the tissue of interest is a cancer tissue.

In embodiments, the clinician can mark the surgical margins as about 0 mm, about 0.25 mm, about 0.5 mm, about 1.0 mm, about 1.5 mm, about 2.0 mm, about 2.5 mm, about 5 mm, about 10 mm, or about 15 mm from the center of the tissue of interest.

For example, if a patient is to be subjected to direct laryngoscopy, the clinician can move the device to the vocal cord tissue for visualization, identify an area that can comprise cancer, and identify surgical margins. For example, the surgical margin can be about 5 mm.

In an embodiment where a patient is to be subjected to an upper GI endoscopy, the clinician can move the device to an anatomical tissue of interest in the esophagus, stomach, or small intestine.

In embodiments wherein a patient is to be subjected to a lower GI endoscopy, the clinician can move the device to an anatomical tissue of interest in the large intestine.

In embodiments where a patient is to be subjected to a laparoscopic procedure, the clinician can move the device to an anatomical tissue of interest inside the patient's body cavity.

CLASSIFYING ANATOMICAL TISSUE AND SURGICAL MARGINS—The Raman probe device as evident from any of the various embodiments disclosed herein can be utilized to classify anatomical tissue and determine adequate surgical margins. In embodiments, when an anatomical tissue of interest is identified and surgical margins (as determined by standard of care) have been marked, the clinician can utilize the any of the various presently disclosed embodiments to acquire a Raman spectrum and assist with classification of the tissue. In some embodiments, the clinician can mitigate any intervening light sources via a switch or by design of the probe tip. Once a Raman spectrum has been acquired, computer analysis can inform the clinician of the classification of the tissue. For example, the method of informing the clinician can comprise an auditory signal, such as a tone, pitch, or statement, or a visual signal such as light flashes. For example, the pitch or the light flash can indicate the presence of cancer or the absence of cancer. The clinician can then use the computer output to inform their decisions regarding adequate surgical margins during tissue resection.

In embodiments the ambient or interfering light can be reduced via a switch within the device. In embodiments, the device can electronically turn off the endoscopic light during Raman spectrum acquisition. In one embodiment, the probe instrument tip design can permit contact with the tissue site. In embodiments, the probe tip design can control the incident laser angle.

In certain embodiments, the clinician can control the incident laser angle by hand, such as via rotation of the Raman probe until the desired incident laser angle is achieved.

An electronic control system can be utilized to engage the Raman analysis while simultaneously switching off any illumination, such as that that can be introduced via a fiberoptic light associated with the endoscope. In embodiments, an electronic switch connects the probe fiber to the Raman spectrometer and a light emitting diode (LED) aiming beam. The housing unit can be configured to contain a momentary switch and circuitry to activate the Raman spectrometer and toggle between the LED source and the incident laser signal. In embodiments, the Raman probe tip is configured such that the tip blocks any ambient or intervening light sources. For example, when the Raman probe tip contacts a specimen, the tip surrounding the Raman probe can block ambient or interfering light (e.g. see FIG. 4A (inset) and FIG. 5.)

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Example 1—Optical Biopsy of Lesions: A Sheath Based Delivery System for Raman Spectroscopy During Flexible Fiberoptic and Rigid Telescopic Laryngoscopy and Pancreatic Cancer Surgery

We disclose herein a Raman spectroscopy system that incorporates a tissue probe into a thin sheath. The design can be adapted to any flexible or rigid laryngoscope. The fiberoptic probe with lens has an outer diameter of approximately 2 mm with a length up to 1.5 m depending on the laryngoscope. The probe will permit laser light delivery and signal collection in real time during endoscopy.

In addition to the optical probe, the spectrometer includes a 785 nm diode laser light source, a dual wave number (800-1800 cm⁻¹ and 2800-3600 cm⁻¹) signal collection system and a computer for signal analysis and display. In embodiments, the signal collection system can collect spectra on a range of about 200 cm⁻¹ to about 4000 cm⁻¹. Photographs of the scopes and designs for the spectrometer and sheath-probe are shown in FIGS. 1-3.

In embodiments, the sheath-based Raman spectroscopy system can be commercially manufactured such that clinicians can routinely use the system in the clinic or the operating room during laryngoscopy.

### Early Diagnosis and Surveillance for Laryngeal Cancer

In an ambulatory setting, clinicians can use the Raman spectroscopy system during flexible laryngoscopy to screen laryngeal lesions for premalignant (keratosis with dysplasia) and malignant (carcinoma in situ, invasive carcinoma) changes. The visual features of these lesions are not diagnostic. Multiple biopsies are often necessary for diagnosis. Raman spectroscopy will simplify post-operative management of laryngeal malignancy during serial follow up laryngoscopy.

### Tumor Mapping and Surgical Margin Detection During Endoscopic Resection of Laryngeal Cancer

Adequate resection is the most significant determinant of success after larynx sparing surgery for carcinoma. Margin detection is especially difficult during endoscopic surgery for malignancy. Deployed on a rigid c, Raman spectroscopy will permit precise margin control during endoscopic laryngeal surgery.

### Tumor Mapping and Surgical Margin Detection During Endoscopic Resection of Pancreatic Cancer

Similar to the laryngeal cancer surgery, the major challenge of the current pancreatic cancer surgery is the inability to remove all existing tumor. With a flexible operative endoscope, Raman spectroscopy will allow the precise intraoperative cancer detection, which aids the complete resection of pancreatic cancer. Raman spectroscopy will be used to detect primary tumor, satellite tumor, tumor margin and surgical bed.

A design that adapts the Raman probe to both flexible and rigid scopes will allow deployment in the clinic for early detection of premalignant and malignant vocal fold lesions and in the operating room for precise intraoperative margin control during endoscopic resection. There are no existing sheath-based designs for flexible laryngoscopy and there are no Raman probe adaptations for rigid operative endoscopy.

### Device Design

The Raman signal is weak and the device has to be carefully designed with enough sensitivity and calibrated with ex vivo specimens. This is a miniaturized probe design for sheath deployment. There are additional challenges with device miniaturization while maintaining signal detection sensitivity and short acquisition times.

We have developed a miniaturized spectroscopy system that can work with endoscopes including sigmoidoscopes, angioscopes, and dental imaging devices using near infrared spectroscopy. The existing technology can be adapted to a Raman spectroscopy system.

See, for example, Li Z, et al. Endoscopic near-infrared dental imaging with indocyanine green: a pilot study. Ann. N.Y. Acad. Sci. 2018; 1421:88-96, which is incorporated herein by reference in its entirety.

### Example 2—Optical Biopsy of Vocal Fold Lesions: Sheath-Based Delivery System During Laryngoscopy

Disclosed herein is a Raman spectroscopy system that can be deployed in an endoscope sheath during laryngoscopy. The device will enable optical diagnosis of laryngeal lesions including carcinoma and accurate margin detection during endoscopic laryngeal tumor resection. The dimensions of the sheath can vary to accommodate different fiberoptic and rigid endoscopes. There are commercially produced sheaths used as microbial barriers during endoscopy and a similar design should accommodate the Raman fiberoptics. There are no prior reports of an endoscopy sheath-probe device with Raman fiberoptics and there are no reports of spectroscopy performed with a rigid endoscope.

### Summary

Early detection of laryngeal cancer significantly increases survival rates and permits more conservative, larynx sparing treatments. It also reduces the length of hospital stay and health care costs¹. The past 20 years have seen a trend towards more conservative, larynx sparing surgery for stage I and II laryngeal carcinoma performed through a trans-oral endoscopic approach². Achieving adequate margins of resection can be challenging. A non-invasive, optical form of biopsy for laryngeal carcinoma will improve early detection, permit more accurate surveillance for recurrence and improve intraoperative margin control.

Raman spectroscopy measures the difference between incident and scattered monochromatic light that is a function of the molecular composition of tissue. Previous studies have demonstrated a spectral difference between normal tissue, benign lesions and carcinoma in ex vivo laryngeal specimens^{3,4}.

Lin, et al. published the only report of in vivo Raman spectroscopy for laryngeal lesions in 2016⁵. They documented spectral discrimination between normal and cancerous tissue during laryngoscopy. Their system used simultaneous 800-1800 cm⁻¹ and 2800-3600 cm⁻¹ wave number analysis. The Raman spectroscopy system included a near infrared diode laser source (785 nm) and a thermo-electric cooled CCD camera. They achieved a diagnostic accuracy of 91.1% (sensitivity—93.3%, specificity—90.1%) for laryngeal cancer. Acquisition times were on the order of milliseconds. They used a 1.8 mm (O.D.) fiberoptic probe with a 1 mm Sapphire ball lens at the distal tip. The Raman probe used during the Lin study was deployed intraoperatively through the operating channel of a flexible laryngoscope. The type and dimensions of the laryngoscope were not specified in their report.

An embodiment described herein is a sheath delivery system that incorporates the Raman probe. The dimensions of the sheath can vary to accommodate different fiberoptic and rigid endoscopes. Embodiments further comprise a disposable sheath design. There are no prior reports of an endoscopy sheath-probe device with Raman fiberoptics and there are no reports of spectroscopy performed with a rigid endoscope.

### Background

### Laryngoscopy

Specialists inspect the vocal folds and other parts of the larynx with specialized endoscopes. Typical indications for laryngoscopy include voice changes, difficulty breathing, dysphagia and hemoptysis. The scopes are available in two basic designs. Flexible scopes allow simple diagnostic evaluation in an un-anesthetized patient. Rigid scopes afford greater magnification and resolution than flexible scopes but are used during operative procedures under general anesthesia performed especially for biopsy and surgical resection of laryngeal lesions. FIG. 4 shows representative flexible and rigid laryngoscopes.

Current video endoscopy systems rely on visual evaluation by the specialist for interpretation. Spectrographic and other optical assessment techniques during laryngoscopy is under-utilized because of cost and deployment difficulties.

### Raman Spectroscopy

When monochromic light from a laser source strikes a substance, photons are absorbed by its surface and reemitted. Most of the reemitted light occurs at the same frequency as the monochromic source (elastic scattering). Depending on the sample, a small portion of the reemitted light will radiate at frequencies above and below the incident frequency (inelastic scattering). Inelastic scattering is dependent upon the molecular structure of the specimen and is termed the Raman effect. One can determine the molecular structure of certain substances using their Raman spectra.

A typical Raman spectroscopy system comprises a laser light source, an optical filter to define the incident frequency and a charge couple device (CCD camera) to detect Raman scattering. Raman spectroscopy developers have incorporated a number of modifications to improve frequency resolution and acquisition times.

Raman spectroscopy is useful in any field where there is a need for a non-destructive chemical analysis or imaging. Engineers use it to detect chemical impurities and manufacturing defects. It also has applications in the environmental science, geology, pharmaceutical and semi-conductor industries.

Advantages include distinct visible spectra for numerous substances, small-required sample size and nondestructive analysis. Not all molecules exhibit Raman effect. For example, Raman spectroscopy cannot be used to analyze metals and alloys. Historically, the most significant disadvantage of Raman analysis has been its long signal acquisition time.

The Raman spectra of tissue specimens represent the weighted some of their macro molecular species and are highly tissue specific. Researchers have identified cellular differentiation in several epithelial types using Raman spectroscopy. In situ applications have been reported in brain⁶, bladder⁷, breast⁸, and skin⁹ neoplasms.

### Market Need

The American Cancer Society estimates that there are 13,000 new cases of laryngeal cancer diagnosed each year¹⁰. Approximately 3700 patients with laryngeal cancer die annually. Early diagnosis and precise control of disease margins are essential for the surgical management of laryngeal carcinoma. Important visual features related to malignancy can be missed on routine laryngoscopy and multiple biopsies are often necessary for diagnosis. The five-year relative survival rate for stage I glottic carcinoma is 90%. This drops to 74%, 56% and 44% for stages II, Ill and IV, respectively. Lack of locoregional control is the most significant cause of surgical failure. Stage I and II lesions are often treatable with larynx sparing endoscopic surgery as long as adequate margins of resection are achieved. Persistent positive margins can require total laryngectomy and/or radiation therapy that might otherwise have been avoided with complete initial resection.

Early detection of malignant and premalignant lesions of the larynx will significantly improve larynx preservation and survival rates after surgical treatment. Disclosed herein is a Raman spectroscopy optical biopsy system that can be used routinely in the clinic or operating room. A design that adapts the Raman probe to both flexible and rigid scopes will allow deployment in the clinic for early detection of premalignant and malignant vocal fold lesions and in the operating room for precise intraoperative margin control during endoscopic resection.

Annual direct healthcare expenditures for laryngeal disease are estimated between 178 and 294 million dollars with 50-70% spent on surgical management¹¹. In the United States there are approximately 10 million medical encounters for laryngeal related symptoms annually. The exact number of laryngoscopies performed is difficult to estimate but it is one of the most commonly performed in-office procedures and most visits to an otolaryngologist with a complaint of vocal dysfunction will be evaluated endoscopically. With sufficient sensitivity and efficient deployment, Raman spectroscopy could become standard practice in adult airway endoscopy.

Our approach to deploying the fiberoptic probe in a sheath allows the system to adapt to any flexible or rigid telescopic laryngoscope. Deployed in-line with existing endoscopes, the device can be used routinely in the clinic or the operating room. There are no similar systems for laryngoscopy in existence.

A commercially manufactured, disposable fiberoptic spectroscopy sheath can be made from materials currently used in medical endoscopy systems. A disposable sheath could be deployed inexpensively. A one-use design avoids infection control, storage and sterilization issues and lends itself to unit pricing in the clinic and operating room. Similar systems such as fiberoptic surgical laser probes have proven attractive to hospital and clinic administrators where the vendor provides the base system free of charge and the user pays only for the disposables kept in inventory.

To our knowledge, there are no similar fiberoptic spectroscopy probe sheaths in development or production. The design to incorporate the probe into a disposable sheath with different dimensions for existing flexible and rigid endoscopes should provide a stable barrier to market competition.

Laryngeal cancer is diagnosed histologically. Many lesions spread in a submucosal plane and multifocal occurrences (“field cancerization”) are common. Identifying the best sites for biopsy can be difficult and many patients undergo multiple procedures for biopsy before a diagnosis is confirmed. Any method that enhances the detection of submucosal tumor will improve biopsy site selection and diagnostic accuracy.

Optical coherence tomography (OCT)¹² is a near-infrared interferometry technique. It has excellent resolution and can image on scales ranging from 10 μm to approximately 10 mm. It is used widely in ophthalmology for retinal imaging. There are also reports of OCT imaging of the larynx and upper airway to measure airway patency. The main disadvantage is cost (approximately $75,000 for a retinal imaging system). OCT imaging is deployed with fiberoptic technology and the same endoscopic sheath delivery system disclosed for use with Raman spectroscopy herein can be used for OCT.

Narrow-band imaging (NBI)¹³ uses blue (415 nm) and green (540 nm) light filtering to enhance the visualization of hemoglobin. It is useful in identifying lesions with enhanced microvascular patterns including neoplasms. Findings are nonspecific but the technique has shown some promise in characterizing neo-angiogenesis in precancerous and cancerous aerodigestive lesions. It is inexpensive and easily deployed in any endoscope. It has proven cost effective in screening endoscopy for colon polyps but success in identifying upper airway lesions has been limited.

Embodiments described herein comprise a 785 nm laser light source, a high-resolution CCD camera, a spectrometer, two filters and a specifically designed fiber with lens. The miniaturized probe will be adapted to a sheath for deployment over the shaft of a flexible or rigid laryngoscope. Various materials for the sheath design can be useful. Constraints on the probe size should be less severe than those encountered in other designs where the probe had to be deployed through an operating channel of the scope. During operation, the camera will image the target tissue, the spectrometer will identify the Raman features and the results will be analyzed and displayed in real time on a computer system.

FIG. 5 illustrates the spectroscopy system and FIG. 6 shows the sheath-probe designs.

### REFERENCES CITED IN THIS EXAMPLE

1. Arriold J, et al. Laryngeal cancer cost analysis: association of case-mix and treatment characteristics with medical charges. Laryngoscope. 2000; 110:1-7.
2. Hoffman H T, et al. Laryngeal cancer in the United States: changes in demographics, patterns of care, and survival. Laryngoscope. 2006; 116(Suppl. 111):1-13.
3. Stone N, Starvroulaki P, et al. Raman spectroscopy for early detection of laryngeal malignancy: preliminary results. Laryngoscope. 2000; 110:1756-1763
4. Lau D P, Huang Z, Lui H, et al. Raman spectroscopy for optical diagnosis in the larynx: preliminary findings. Lasers in Surgery and Medicine. 2005; 37:192-200.
5. Lin K, et al. Real-time in vivo diagnosis of laryngeal carcinoma with rapid fiber-optic Raman spectroscopy. Biomedical Optics Express. 2016; 7(9):3705-3715.
6. Jermyn M, et al. Intraoperative brain cancer detection with Raman spectroscopy in humans Sci Transl Med. 2015; 7:274ra19.
7. Li, S. et al. Characterization and noninvasive diagnosis of bladder cancer with serum surface enhanced Raman spectroscopy and genetic algorithms. Sci. Rep. 2015; 5:1-7.
8. Haka A S, et al. Diagnosing breast cancer using Raman spectroscopy: prospective analysis. J Biomed Opt. 2009; 14(5):054023 1-8. Published online 2009 Oct. 14. doi: 10.1117/1.3247154.
9. Zhao J, Lui H, McLean D I, Zeng H. Real-time Raman spectroscopy for non-invasive skin cancer detection—preliminary results. Conf Proc IEEE Eng Med Biol Soc. 2008; 2008:3107-9.
10. American Cancer Society (2017). Survival rates for laryngeal and hypopharyngeal cancers by stage. Retrieved from https://seer.cancer.gov/statfacts/html/larynx.html
11. Cohen S M, et al. Direct health care cost of laryngeal diseases and disorders. Laryngoscope. 2012; 122(7): 1582-1588.
12. Endoscopic optical coherence tomography: technologies and clinical applications [Invited]. Biomedical Optics Express. 2017; 8(5); 1-40.
13. Piazza C, Del Bon F, Peretti G, and Nicolai P. Narrow band imaging in endoscopic evaluation of the larynx. Curr Opin Otolaryngol Head Neck Surg. 2012; 20:472-476.

### Example 3

### Determination of Histologic Changes Due to Raman Spectrum Measurement

To determine if there are any histologic effects of the incident laser light on tissue, we performed Raman spectrographic analysis on fresh tonsil tissue. With IRB approval we studied the tonsils of 8 patients undergoing tonsillectomy for obstructive sleep apnea with tonsillar enlargement. A total of 61 specimens were subjected to a range of laser (785 nm) powers and exposure times. Total energy exposure of the tissue samples ranged from 0 (controls) to 4.6 Joules (230 milliwatts×20 seconds exposure). The pathologist, who was blinded to the exposure settings, graded each specimen histologically for thermal change. Findings were graded from 0 (no fields with histologic change) to 5 (100% of fields with thermal change). Using logistic regression, we determined that there was no significant tissue alteration compared to controls at any total laser energy level (p=1.6×10-6). The results demonstrate that adequate energy can be delivered to detect Raman scattering in tissue samples without sample degradation.

### Raman Spectroscopy Ex-Vivo Laryngeal Carcinoma Margins Study

We have obtained IRB approval to begin an ex-vivo study of human laryngeal carcinomas that are resected endoscopically. The study will be completed at Our Lady of the Lake Hospital in Baton Rouge. After establishing spectral comparisons between documented malignant and non-malignant margins, we will complete a double-blind study comparing Raman spectrometry predictions to the histologically determined margin status on ex-vivo specimens. Without wishing to be bound by theory, the device disclosed herein can be utilized for intraoperative margin detection during endoscopic resection of upper aerodigestive cancers.

We completed our first Raman examination of a laryngeal resection specimen. Superficial (grossly positive) and deep (grossly negative) margins were evaluated in a patient with a history of laryngeal carcinoma-in-situ.

### Endoscopic Delivery Systems for Raman Spectrometry

We have designed and prototyped an inexpensive sheath-based delivery system for single fiber Raman spectrometry suitable for upper airway endoscopy. We are also designing a handheld probe for fiber deployment during suspension laryngoscopy that will improve resolution by eliminating signal degradation along the fiber.

We are working to design a system that can deliver the incident laser light and detect the Raman system in the absence of interfering ambient light. For an in-vivo system we can accomplish this by using the delivery probe to electronically turn off the endoscopic light during the brief period of spectrum measurement. For the ex-vivo carcinoma margins study we are using an enclosure to eliminate ambient light.

### Example 4—Raman Spectroscopy Ex-Vivo Laryngeal Carcinoma Margins Study Protocols

Endoscopic resections will be performed and the specimen orientation will be tracked (FIG. 7). In some cases, there can be more than one specimen per procedure; each specimen will be kept in the proper orientation.

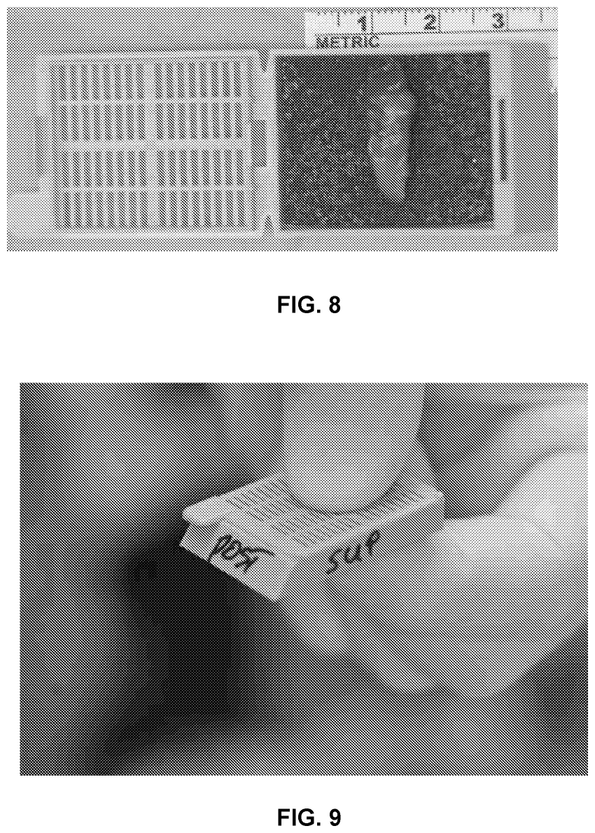

In the operating room, the specimen will be placed on a histology cassette with the orientation noted (FIG. 8). When closed, the edges of the cassette will be labeled with the peripheral margins (FIG. 9). The deep margin faces the bottom of the cassette.

The cassette will be taken to a workspace just outside of the OR where the engineering team will be waiting to complete the study on the specimen. This is a large room with plenty of space for equipment and personnel. The engineering team can setup in this area without observing OR protocol; this is a non-sterile area without patient contact. The orientation of the specimen must be maintained whenever the cassette is opened. The specimen will have a unique patient identifier.

During the Raman study, the clinical group will explain the specimen orientation and margins. We should perform Raman spectroscopy on the four peripheral margins, the deep margin and the central tumor. The spectra should be labeled with the patient identifier appended with the name of the margin on the cassette for peripheral margins or with “deep” or “tumor” depending on the specimen location. After Ramen spectroscopy analysis, the specimen cassette will be closed, transferred to the specimen processing room, and processed according to hospital/pathology protocol.

Each case takes approximately 1-2 hours to complete the resection. The Raman processing will take approximately 15 min.

### Example 5—a Machine-Learning-Assisted Raman Spectroscopy System Designed for Early Detection and Surgical Margin Control in Laryngeal Carcinoma

### Abstract

Laryngeal carcinoma is the second most common cancer of the respiratory tract with an overall 5-year survival rate of 60%. Clinical stage at the time of diagnosis and margin status during surgical resection are the two most important factors correlated with disease prognosis. Five-year survival drops from approximately 90% for tumors confined to the larynx, to 60% and 27% for regional and distant disease, respectively. Early diagnosis depends upon careful assessment of suspicious laryngeal lesions noted on diagnostic laryngoscopy. Confirmation requires biopsy and histologic evaluation. Laryngeal tissue sampling is complicated by the need for endoscopic instrument placement in the airway and the heterogeneous appearance of premalignant and malignant lesions. Margin detection during surgical resection of laryngeal carcinoma also requires histologic assessment that is highly dependent on surgeon selected sample sites and the tendency for submucosal tumor extension. Nationally, the incidence of positive margins following endoscopic resection of laryngeal carcinoma is 22%.

An accurate method of optical biopsy during laryngoscopy would enable real-time classification of laryngeal lesions. Disclosed herein is a Raman spectroscopy imaging system that will mitigate the problem of delayed diagnosis and high incidence of positive margins following tumor resection. We will build a Raman spectrometer optical system and probe designed for laryngoscopy. Data from our laboratory and clinical studies show that Raman spectroscopic imaging of laryngeal pathology is safe and can distinguish between normal and cancerous tissue. This project will increase our understanding of the spectral characteristics of laryngeal cancer, extend lesion differentiation to include mucosal dysplasia and carcinoma in situ, and facilitate rapid cancer detection using an endoscopic imaging approach. This research will reduce morbidity and improved survival rates in laryngeal cancer. Principles and methods derived from the study will also be applicable to other forms of head and neck cancer.

### Project Narrative

Laryngeal carcinoma is one of the most devastating forms of cancer, even in patients who are ultimately cured of the disease. Failure to efficiently detect laryngeal carcinoma leads to diagnostic delays, incomplete tumor resection during surgical management and poor outcomes. This project will solve problems related to delayed diagnosis and inadequate surgical removal of laryngeal carcinoma using an innovative optical biopsy technique based on spectroscopic imaging.

Early diagnosis and adequate resection with clear tumor margins are crucial for local control and disease-free survival following the treatment of laryngeal cancer. Visual estimation of surgical margins is subject to errors related to heterogeneous tumor appearance and submucosal extension. Frozen section analysis only determines the margin status at discrete points along the lesion. There is a need for early diagnosis and improved intraoperative margin detection for carcinoma of the larynx.

To improve early diagnosis and tumor margin determination in laryngeal carcinoma, disclosed herein is a Raman spectroscopy optical system for laryngoscopy. This approach is based on published literature and results from our laboratory that show Raman spectral differences between tumor and non-cancerous tissue. Without wishing to be bound by theory, real-time detection of normal tissue, dysplasia, carcinoma in situ and invasive carcinoma will substantially lower the incidence of delayed diagnosis and incomplete tumor resection. The benefit will be lower rates of local and regional recurrence, reduced need for postoperative radiation therapy and fewer organ sacrificing procedures for advanced disease.

### Build a Machine-Learning-Assisted Raman Imaging System for Laryngoscopy.

Develop spectroscopy hardware optimized for the detection of laryngeal carcinoma.

We will construct a Raman system for laryngeal cancer detection by optimizing its optical configuration. Using current designs, we will also construct a laryngoscopy probe to detect tumor tissue and map surgical margins. The outcome will be a system capable of rapidly capturing Raman inelastic scattering across frequencies useful for in vivo use during laryngoscopy.

Develop machine-learning-assisted signal processing algorithms for Raman spectroscopic cancer detection.

Machine learning signal processing software can be utilized to classify Raman spectral features. This includes new multi-modality neural networks and a murine-human hybrid, interspecies transfer learning model. The outcome will be Raman specific algorithms to classify normal tissue, dysplasia, carcinoma in situ and invasive carcinoma in the human larynx.

### Evaluate the Raman Imaging System for In Vivo Accuracy.

Perform initial system optimization and train a baseline neural network for transfer learning using mice.

We will validate the system on a murine cancer model by analyzing spectral differences between normal tissues and murine head and neck cancer xenografts. We will fine tune system parameters for in vivo testing in humans and accumulate data for neural network training in a transfer learning model.

Evaluate the Raman imaging system in patients with laryngeal carcinoma undergoing laryngoscopy.

We will evaluate the system in patients diagnosed with stage I/II laryngeal carcinoma. In this clinical study, we will compare tumor classifications performed by our Raman spectroscopic imaging system to their histologic findings. We will prepare the system for clinical trials and commercialization.

### Impact

The study will establish innovative new technology to overcome the current limitations of tumor identification and margin control in head and neck cancer. It will also provide new insights into current approaches to the diagnosis and surgical management of laryngeal carcinoma. Without wishing to be bound by theory, the impact will be decreased morbidity and improved survival rates in laryngeal cancer.

### Research Strategy

### Significance

Tumor identification and surgical margin control in endoscopic laryngeal carcinoma resection.

The past 20 years have seen a trend towards more conservative, larynx sparing surgery for stage I and II laryngeal carcinoma performed through a trans-oral endoscopic approach^{1,2}. For optimal management, the surgeon must accurately distinguish between normal mucosa, dysplasia, carcinoma in situ and invasive carcinoma. Surgical margin status is the most important prognostic factor in laryngeal cancer managed with primary surgery³.

In 2019, a national cancer database survey of early laryngeal carcinoma treated with trans-oral laser resection showed an alarming 22% incidence of positive margins⁴. In 2017 a review of outcomes related to positive margins in endoscopic laser resection of early laryngeal cancer showed that positive or close margins reduced 5-year survival in laryngeal cancer from 85.1% to 61.3%⁵. There was a significant increase in the recurrence rate and need for postoperative radiation therapy and revision surgery. The system disclosed herein will allow the surgeon to distinguish invasive carcinoma from other mucosal changes and map accurate tumor margins in real time during endoscopic laryngeal carcinoma resection.

### Clinical Applications of Raman Spectroscopy.

Raman spectroscopy (RS) detects the small fraction of inelastic scattering of photons from an incident monochromatic light source⁶. A RS system integrates a laser light source for sample excitation, a charge-coupled device sensor, an optical analysis module and a fiberoptic transmission channe^{l7}. It provides a highly specific, structural fingerprint of chemical samples.

A number of chemical species of biomedical interest produce Raman scattering, including proteins and nucleic acids. The Raman spectra of tissue specimens represent the weighted sum of their macromolecular species and are highly tissue specific. Researchers have identified cellular differentiation in several epithelial types using Raman spectroscopy. Prior analyses of laryngeal lesions have been ex vivo studies^{8,9,10,11}. Tissues studied include normal laryngeal mucosa, dysplasia, carcinoma and squamous papilloma. When correlated with histologic results, reported lesion detection sensitivities for carcinoma ranged from 69 to 92%. Specificities ranged from 90 to 94%. Other ex vivo studies have been reported in brain¹², bladder¹³, breast¹⁴ and skin¹⁵ neoplasms. Due to the technical constraints of current RS systems, in vivo studies are limited. Preliminary reports of in vivo RS have documented its ability to detect cancer in gastrointestinal, breast, brain, skin and cervical tumors¹⁶.

There are significant challenges to in vivo RS implementation¹⁶. Most RS systems are deployed in the laboratory and, until recently, these devices have occupied large, non-portable footprints. The quantum yield of RS scattering is weak, representing approximately 1 in 107 incident photons. Biomolecules are prone to fluorescence upon monochromatic illumination with a photon yield in 1-10% range^{17,18}, resulting in significant signal-to-noise ratio reduction. RS system designers often compensate for fluorescent spectral interference with increased laser energy, longer exposure times, mechanical sample stabilizers and thermoelectric cooling systems¹⁹. These signal enhancement methods will require careful engineering before they can be applied in an in vivo setting. Streamlined designs for clinical application will need to maintain laser light source power, optical alignment, an intact transmission pathway and instrument sensitivity.

### Raman Spectroscopy and Endoscopic Optical Biopsy.

Microscopic evaluation of hematoxylin-eosin stained biopsy samples remains the standard for tumor diagnosis and margin detection in head and neck cancer. However, histopathologic analysis relies on a subjective assessment of tissue morphology and is subject to sampling error. Many lesions spread in a submucosal plane and multifocal occurrences are common. Identifying the best sites for biopsy can be difficult and many patients undergo multiple biopsy procedures before a diagnosis is confirmed.

Our goal is to design and implement a RS system capable of rapidly distinguishing cancerous from non-cancerous tissue during airway endoscopy. Ultimately, Raman spectroscopic characterization of normal, precancerous and cancerous tissue at a molecular level can be more relevant than routine histopathology.

### Significance

Current impediments to routine clinical use of Raman spectroscopy include limited anatomical access, the design constraints listed above, and the need to control the lighting environment in the region of interest. Unlike other approaches to biomedical Raman spectroscopy, we are designing a system to specifically address these issues. Upon successful completion of our specific aims, we will be prepared to begin clinical trials of a Raman spectroscopy system that can perform non-invasive optical biopsy of suspicious laryngeal lesions and map surgical margins during endoscopic resection of laryngeal carcinoma.

### Innovation

Raman spectroscopy can be useful to identify cancer by detecting tissue changes at the molecular level. However, there are limited reports of RS used to detect cancer and many of the existing studies cannot be replicated^{8,9,20,21,22}. This can be attributed to the complicated implementations of spectrometer hardware, delivery systems that cannot be adapted to clinical use and a limited number of analysis algorithms^{16,23,24}. This project will advance the use of RS for the detection of laryngeal carcinoma. Knowledge and techniques gained from this disclosure are also applicable in other clinical applications of RS.

### Raman Spectrometer Optimizations for In Vivo Use.

In vivo spectrometer deployment will require portability and efficient power consumption while maintaining instrument sensitivity. Also, the total laser energy is limited by potential tissue alterations resulting in damage to normal structures and interference with histologic evaluation of tissue samples²⁵.

We are solving the in vivo spectrometer deployment issues with innovative optimizations of the optical parameters and sensor design. We have successfully used a portable Raman spectrometer prototype that can be moved to the operating room. Initial studies of ex vivo laryngeal cancer resection specimens have shown differences in the spectra of normal tissue and carcinoma using incident laser wavelengths and power settings suitable for in vivo use (see data described herein).

### Raman Spectroscopy Probe Designed for Laryngoscopy.

Standard probes used for Raman spectroscopy in the laboratory for chemical analysis are not suitable for use in human tissue studies^{16,23,24}. Important parameters include designs for endoscopic placement, sterilization, and precise probe positioning in the field of study. RS studies performed in the laboratory eliminate the effects of ambient light on recordings by darkening the environment. The incident laser angle is precisely controlled by mechanical fixation of the sample and probe. While effective for laboratory use, these designs cannot be applied in a clinical setting.

We have designed an instrument specifically for in vivo use of RS during laryngoscopy. The probe includes an innovative design of the instrument tip to allow precise contact with the tissue site and control of the incident laser angle. It also eliminates ambient lighting effects. An electronic control system triggers the Raman analysis while simultaneously switching off the fiberoptic illumination. The design is intended for the real-time detection of laryngeal mucosal tissue changes and surgical margin mapping during the endoscopic excision of laryngeal carcinoma.

Machine learning assisted analysis of Raman signals to differentiate normal mucosa, dysplasia, carcinoma in situ and carcinoma of the larynx.

Investigators analyze Raman spectra using standard signal processing techniques based on one or two spectral peaks. Although there are studies showing single peak differences in signals from normal and cancerous tissue, there does not appear to be sufficient sensitivity for clinical application.

Our group discloses Raman specific machine learning algorithms^{26,27} to detect multiple spectral features and differentiate normal from cancerous/precancerous tissue. Inspection of our ex vivo laryngectomy data indicate that there is sufficient information to train and deploy a convolutional neural network for autonomous classification of these lesions.

We are exploring deep learning technology using multimodality networks that combine spectral data with endoscopic imaging. We have also designed an innovative interspecies transfer learning model where the base network is trained on a large murine Raman spectrum dataset and fine tuned to a limited set of human spectra.

### Approach

Our overall strategy starts with our existing experience with clinical Raman spectroscopy. We will build and optimize the in vivo Raman system using the knowledge gained during the build of our current device. Data acquired during the initial phases of the project will enable us to create advanced machine learning algorithms capable of real time classification during laryngoscopy. After validating the overall system design we can proceed with clinical testing without trials to compare in vivo Raman spectroscopy tissue classification with histologic findings in patients undergoing endoscopic surgery for laryngeal carcinoma.

### Preliminary Data

### Raman Laser Safety.

Prior to using the 785 nm Raman laser to analyze laryngeal specimens, we completed a controlled study of its histologic effects on fresh tonsillar tissue over a range of incident laser power (0-230 Mw) and exposure times (1-20 sec). The presence or absence of thermal tissue damage was assessed on histologic sections. Logistic regression analysis confirmed no histologic changes related to Raman laser irradiation at energies used to analyze human tissues.

### Ex Vivo Evaluation of Laryngeal Carcinoma and Tumor Margins.

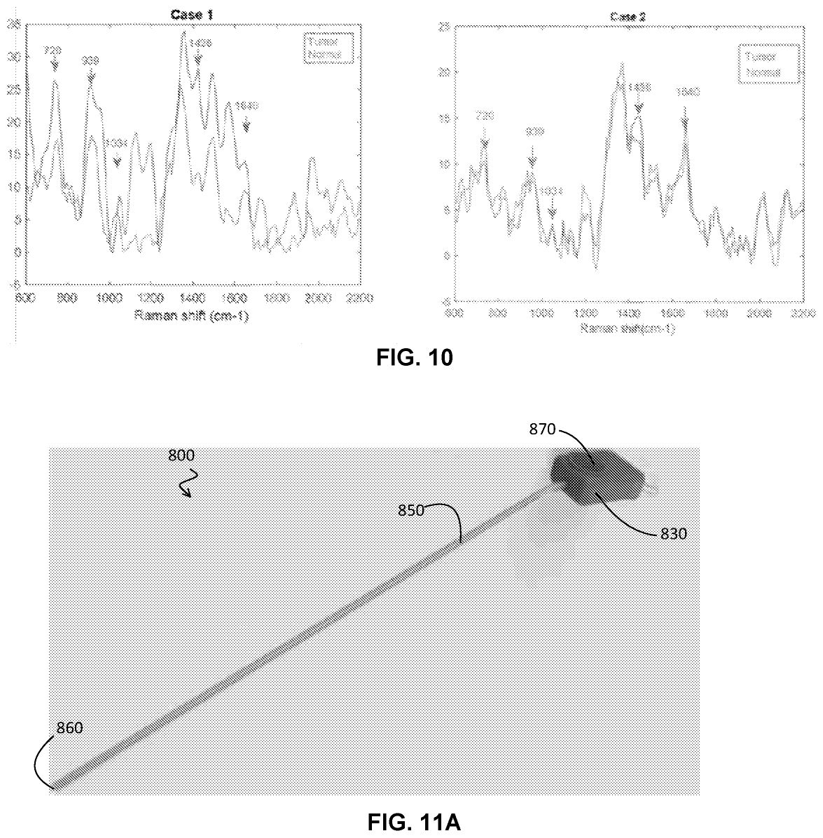

In an IRB approved study of ex vivo laryngeal carcinoma resection specimens, we evaluated tumor and non-cancerous margins using a 785 nm Raman imaging system. A single fiber system was deployed in an enclosed stage for specimen stabilization and ambient light exclusion. The Raman spectroscopy findings were compared to the reported pathology. Differences in Raman shift between cancerous and non-cancerous sites were most pronounced in the 939 and 1438 nm peaks. These correspond to increases in protein C—C and lipid CH2-CH3 molecular bonds, respectively 24. FIG. 10 shows two representative spectra.

### Build a Machine Learning Assisted Raman Imaging System for Applications in Head and Neck Cancer.

### Develop Spectroscopy Hardware Optimized for the Detection of Laryngeal Carcinoma.

We will improve the current design of our Raman spectrometer by optimizing the optical parameters and components. Optimizations will improve tissue detection sensitivity and enable in vivo use.

All reported Raman systems for cancer detection use incident lasers in the near-infrared region I (NIR I, 700-1000 nm) to reduce the tissue auto-fluorescence prevalent in the 300-726 nm region^{28,29,30}. However, Raman signal intensity is proportional to 2-4 and the spatial resolution is proportional to 2. The Raman signal intensity excited by a 785 nm laser is only 21.1% of that excited by a 532 nm laser at the same power. We will evaluate designs with different wavelengths, from UV (244 nm, 257 nm) to visual (532 nm, 633 nm) to NIR II (1064 nm)^{30,31}. UV Raman spectroscopy can provide better Raman signal intensity and auto-fluorescence suppression¹⁸. NIR II Raman spectroscopy provides better auto-fluorescence suppression than NIR I³², but needs a indium gallium arsenide (InGaAs) spectrometer. InGaAs technology is available in our laboratory and our group has experience in NIR I and II imaging^{33,34}.

To explore the laser wavelengths, we will use chemical systems. These include a large Renishaw in Via Reflex Raman confocal microscope operating with laser wavelengths of 532, 633 and 785 nm. We will use biological waste tissues for examination under various excitation wavelengths to compare the imaging performance, including the Raman signal-to-background ratio (SBR). Although the equipment is not suitable for in vivo tissue diagnosis, the test results will enable us to screen wavelengths that are useful for our portable system.

Appropriate filters can be important in separating the Raman signals from the Rayleigh signals (elastic scattering). We will use a high-optical-density (OD) band-pass filter for the excitation signal and a high-OD long-pass filter to prevent laser excitation light from reaching the sensor. Lasers with different wavelengths will be coupled with different optical filters to improve the detection sensitivity. A silicon-based spectrometer will be used for UV, visible and NIR-I Raman spectroscopic imaging; an InGaAs based spectrometer will be used for NIR II studies. The laser power will be tailored to achieve optimal Raman signals without thermal tissue damage.

A visible spectrum illumination beam will help to determine the optimal distance and angle for the incident laser. We will calibrate the relationship of beam size and shape to the distance and angle between the sample and Raman probe tip. The calibration data will enable us to calculate the optimal laser incidence distance (ranging in 0-10 mm) to achieve the best Raman SBR for the tissue detection. For clinical applications, we will need to optimize system design to enable exposure times less than 5 seconds.

Using our CAD models for Raman fiber deployment, we will refine and construct an instrument that providers can use in the larynx to detect tumor and map surgical margins. The design allows for the delivery of the Raman probe including connectors for the spectrometer; switchable fiberoptic lighting of the surgical field; and an actuator to turn off the visible light source, activate the incident Raman laser and record the spectrum. Our objective is to create a prototype suitable for clinical testing.

An exemplary surgical probe for direct laryngoscopy and carcinoma margin detection is shown in FIG. 2. The design comprises a tubular structure with a channel to carry the Raman fiberoptic cable to its distal end. The distal tip creates a controlled offset (see the inset of FIG. 4A and FIG. 5) between the tissue interface and the end of the fiber. An electronic switch connects the probe fiber to the Raman spectrometer and a light emitting diode (LED) aiming beam. The housing unit contains a momentary switch and circuitry to activate the Raman spectrometer and toggle between the LED source and the incident laser signal.

The probe assembly and controller housing will be custom manufactured. The stainless-steel probe tubing, control unit electronics, fiberoptic cable and LED components are commercially available. Design parameters, including the fiber cable diameter, housing materials, and tip offset distance, can be manipulated based on the standard of care, the needs of the patient, or the preference of the clinician. Embodiments can be produced using 3D printed models, injection molding manufacturing, or a combination thereof.

### Develop Machine-Learning-Assisted Signal Processing Algorithms for Raman Spectroscopic Cancer Detection.

Machine-learning-assisted signal processing software can be utilized to classify the Raman spectral features and differentiate cancerous, precancerous and normal laryngeal mucosal tissues. A range of machine learning (ML) algorithms can be employed, including deep neural networks (DNNs).

Deep neural networks have become the focus of recent machine ML research and have shown improved accuracy across a range of classification tasks. This research will develop convolutional neural networks (CNNs) to classify the tissues. Different CNN architectures will be evaluated for optimal classification. As is often the case in medical applications, limited training data will pose the main challenge in the development of our Raman spectral tissue classifier³⁵. Without being bound by theory, to alleviate the need for data, three strategies can be employed to enhance training, reduce overfitting and improve generalization—regularization, transfer learning and multimodality networks.

Regularization has been shown to improve ML model performance³⁶. For deep neural networks, in addition to parameter regularizations, we can regularize a neural network through pre-training or co-training. One of our research group members has developed co-trained deep neural networks using auxiliary generative networks. These are pre-trained or concurrently trained, multitasked systems that are combined with the target network. The two neural networks share common convolutional layers. By regularizing the target classifier with the generative auxiliary network, we have improved classification accuracy with reduced overfitting. In previous work, we have used this strategy for cell type classification using one-dimensional DNA sequence information and for mammogram image classification with two-dimensional data and a generative adversarial network (GAN)^{37,38}. We will extend these designs to the spectral-based tissue classification and explore different auxiliary networks and tasks to reduce overfitting and enhance network accuracy.

Transfer learning is a common practice in DNNs designed for medical image analysis³⁹. A neural network that was previously trained on non-specialized images is fine-tuned to the specific application domain. For the one-dimensional spectral data, it can be difficult to find an existing, pre-trained model. Our research will focus on a different transfer learning paradigm. We will train a neural network model using a large mice dataset and then fine-tune the model using human data. Without wishing to be bound by theory, this cross-species transfer learning model to outperform models trained directly from human tissue spectra and transfer learning models based on common environmental images (e.g. ImageNet).

A multi-modality DNN integrates inputs of different types and classifies based on the joint information. For our classification task, in addition to the spectral signal, digital photographic images are also available. Without being bound by theory, neural networks can be developed that process the two different types of information jointly and perform classification based on the combined features. Members of our research group have developed multi-modality neural networks for recommender systems⁴⁰ and CNN classification systems for endoscopic images⁴¹.

Software for data processing and neural network training can be developed. Without being bound by theory, the software can be implemented in Python and use TensorFlow or other deep learning frameworks for neural network computation. Once a ML model with satisfactory sensitivity and specificity is trained, the model will be deployed for real time processing of Raman spectral data. Without wishing to be bound by theory, a computational system capable of distinguishing normal, dysplasia, carcinoma in situ and invasive carcinoma using Raman spectra obtained during laryngoscopy will be developed.

### Evaluate the Raman Imaging System Developed for In Vivo Accuracy.

### Perform Initial System Optimization and Train a Baseline Neural Network for Transfer Learning Using Mice.

We can study the Raman imaging system disclosed herein using a head and neck cancer mouse model that has been developed by our group. Fifty-five athymic mice (Nu/J) will be implanted with human squamous cell carcinoma cells (FaDu)⁴². The tumors and surrounding normal tissues will be imaged. During this study we will perform initial optimization of the system optical parameters and collect data for the neural network classifier. The full description, including protocol, is included in the Vertebrate Animals section.

Evaluate the Raman Imaging System in Patients with Laryngeal Carcinoma Undergoing Laryngoscopy.

After development of the in vivo Raman spectroscopy system for laryngoscopy, we can pursue an IRB approved, in vivo study of the system without clinical trials. A series of 30 patients with stage I or II laryngeal carcinoma will be enrolled with consent. We will evaluate the diagnostic and surgical margin detection accuracy during laryngoscopy. Diagnosis and margin status determined spectroscopically will be compared to the histologic findings. This will be an internally controlled, single cohort study with no influences on patient diagnosis or surgical margin selection. The full protocol is described in the Human Subjects section.

### Data Interpretation

Multiclass observations from the diagnostic study (normal, dysplasia, carcinoma in situ, carcinoma) and binary classification (negative or positive) in the case of the surgical margin study will be summarized in their respective confusion matrices. Observations will be reported in terms of specificity, sensitivity, precision and overall performance (accuracy and F score).

### Projected Future Directions

Our long-term goal is a clinical trial of a Raman spectroscopy system tuned for head and neck cancer evaluation. Following successful clinical testing of the laryngeal system, we plan to design additional instruments and algorithms for cancer detection. This includes designs for the early detection of oral cavity, pharyngeal and laryngeal cancers in the clinic.

### REFERENCES

1. Hoffman H T, Porter K, Karnell L H, et al. Laryngeal cancer in the United States: changes in demographics, patterns of care, and survival. Laryngoscope. 2006; 116(Suppl. 111):1-13.
2. Eckel H E, Perretti G, Remacle M, Werner J. Endoscopic Approach. In: Remacle M., Eckel H. (eds). Surgery of Larynx and Trachea. Berlin, Heidelberg: Springer, 2009.
3. Zhang S Y, Lu Z M, Luo X N, et al. Retrospective analysis of prognostic factors in 205 patients with laryngeal squamous cell carcinoma who underwent surgical treatment. PLOS ONE. 2013; 8(4): e60157. doi: 10.1371/journal.pone.0060157.
4. Hanna J, Brauer P R, Morse E, Mehra S. Margins in laryngeal squamous cell carcinoma treated with transoral laser microsurgery: a national database study. Otolaryngology-Head and Neck Surgery. 2019; 161(6): 986-992.
5. Fiz I, Mazzola F, Fiz F, et al. Impact of close and positive margins in transoral laser microsurgery for Tis-T2 glottic cancer. Front Oncol. 2017; 7:245. doi: 10.3389/fonc.2017.00245.
6. Gardiner D J, Graves P R, Bowley H J. Practical Raman Spectroscopy. Berlin: Springer-Verlag, 1989.
7. Wang C, Zeng L, Li Z, Li D. Review of optical fiber probes for enhanced Raman sensing. J. Raman Spectrosc. 2017; 48:1040-1055. doi: 10.1002/jrs.5173.
8. Lau D P, Huang Z, Lui H, et al. Raman spectroscopy for optical diagnosis in the larynx: preliminary findings. Lasers Surg Med. 2005; 37:192-200. doi: 10.1002/1sm.20226.
9. Stone N, Stavroulaki P, Kendall C, et al. Raman spectroscopy for early detection of laryngeal malignancy: preliminary results. Laryngoscope. 2000; 110(10 Pt 1):1756-1763. doi:10.1097/00005537-200010000-00037.
10. Lin K, Cheng D L P, Huang Z. Optical diagnosis of laryngeal cancer using high wavenumber Raman spectroscopy. Biosens Bioelectron. 2012; 35(1):213-217. doi:10.1016/j.bios.2012.02.050.
11. Teh S K, Zheng W, Lau D P, Huang A. Spectroscopic diagnosis of laryngeal carcinoma using near-infrared Raman spectroscopy and random recursive partitioning ensemble techniques. Analyst. 2009; 134: 1232-1239.
12. Jermyn M, Mok K, Mercier J, et al. Intraoperative brain cancer detection with Raman spectroscopy in humans. Sci Transl Med. 2015; 7(274ra19): 7-9.
13. Li S, Li L, Zeng Q, et al. Characterization and noninvasive diagnosis of bladder cancer with serum surface enhanced Raman spectroscopy and genetic algorithms. Sci Rep. 2015; 5:1-7.
14. Haka A S, Volynskaya Z, Gardecki J A, et al. Diagnosing breast cancer using Raman spectroscopy: prospective analysis. J Biomed Opt. 2009; 14(5):0540231-8. doi: 10.1117/1.3247154.
15. Zhao J, Lui H, McLean D I, Zeng H. Real-time Raman spectroscopy for non-invasive skin cancer detection—preliminary results. Conf Proc IEEE Eng Med Biol Soc. 2008; 2008:3107-9.
16. Cui S, Zhang S, Yue S. Raman spectroscopy and imaging for cancer diagnosis. J Healthc Eng. 2018; 2018:8619342. doi: 10.1155/2018/8619342.
17. Boyaci I H, Genis H E, Guven B, et al. A novel method for quantification of ethanol and methanol in distilled alcoholic beverages using Raman spectroscopy. Journal of Raman Spectroscopy 2012; 43:1171-1176.
18. Haustein E, Schwille P. Trends in fluorescence imaging and related techniques to unravel biological information. HFSP J. 2007 September; 1(3):169-180.
19. Kuhar N, Sil S, Verma T, Umapathy S. Challenges in application of Raman spectroscopy to biology and material. RSC Adv. 2018; 8:25888-25908.
20. Pandya A K, Serhatkulu G K, Cao A, et al. Evaluation of pancreatic cancer with Raman spectroscopy in a mouse model. Pancreas. 2008; 36:e1-e8.
21. Lin K, Zheng W, Lim C M, Huang Z. Real-time in vivo diagnosis of laryngeal carcinoma with rapid fiber-optic Raman spectroscopy. Biomedical Optics Express. 2016; 7:3705-3715.
22. Eberhardt K, Stiebing C, Matthaeus C, et al. Advantages and limitations of Raman spectroscopy for molecular diagnostics: an update. Expert Rev Mol Diagn. 2015; 15:773-787.
23. Cordero E, Latka I, Matthaus C, et al. In-vivo Raman spectroscopy: from basics to applications. J Biomed Opt. 2018; 23:071210.
24. Auner G W, Koya S K, Huang C, et al. Applications of Raman spectroscopy in cancer diagnosis. Cancer Metastasis Rev. 2018; 37:691-717.
25. Thomsen S. Pathologic analysis of photothermal and photomechanical effects of laser-tissue interactions. Photochem Photobiol. 1991; 53:825-835.
26. Lakhani P, Gray D L, Pett C R, et al. Hello world deep learning in medical imaging. J Digit Imaging 2018; 31:283-289.
27. Yamashita R, Nishio M, Do R K G, Togashi, K. Convolutional neural networks: an overview and application in radiology. Insights into Imaging 2018; 9:611-629.
28. Haussinger K, Becker H, Stanzel F, et al. Autofluorescence bronchoscopy with white light bronchoscopy compared with white light bronchoscopy alone for the detection of precancerous lesions: a European randomised controlled multicentre trial. Thorax. 2005 June; 60(6):496-503. doi: 10.1136/thx.2005.041475.
29. Krauss E, Agaimy A, Douplik A, et al. Normalized autofluorescence imaging diagnostics in upper GI tract: a new method to improve specificity in neoplasia detection. Int J Clin Exp Pathol. 2012; 5(9):956-964.
30. Smith A M, Mancini M C, Nie S. Bioimaging: second window for in vivo imaging. Nat Nanotechnol. 2009; 4:710-711.
31. Renkoski T E, Banerjee B, Graves L R, et al. Ratio images and ultraviolet C excitation in autofluorescence imaging of neoplasms of the human colon. J Biomed Opt. 2013; 18:016005.
32. Yang S, Akkus O. Fluorescence background problem in Raman spectroscopy: Is 1064 nm excitation an improvement of 785 nm? https://www.ccmr.cornell.edu/wp-content/uploads/sites/2/2015/11/FL-Time-1064-nm-vs-785-nm-Shan-9_30. pdf. Accessed 9.28.2020.
33. Li Z, Zaid W, Hartzler T, et al. Indocyanine green-assisted dental imaging in the first and second near-infrared windows as compared with X-ray imaging. Ann NY Acad Sci. 2019; 1448:42-51.
34. Li Z, Holamoge Y V, Li Z, et al. Detection and analysis of enamel cracks by ICG-NIR fluorescence dental imaging. Ann NY Acad Sci. 2020; 1475(1):52-63.
35. Shaikhina T, Khovanova N A. Handling limited datasets with neural networks in medical applications: A small-data approach. Artif Intell Med. 2017; 75:51-63.
36. Girosi F, Jones M, Poggio T. Regularization theory and neural networks architectures. Neural Computation. 1995; 7(2):219-269.
37. Shams S, Platania R, Kim J, et al. A distributed semi-supervised platform for DNase-seq data analytics using deep generative convolutional networks. Proceedings of the 2018 ACM International Conference on Bioinformatics, Computational Biology, and Health Informatics. 2018:244-253.
38. Shams S, Platania R, Zhang J, et al. Deep generative breast cancer screening and diagnosis. International Conference on Medical Image Computing and Computer-Assisted Intervention (MICCAI). 2018:859-867.
39. Zhou Z, Shin J, Zhang L, et al. Fine tuning convolutional neural networks for biomedical analysis: actively and incrementally. 2017 IEEE Conference on Computer Vision and Pattern Recognition (CVPR). 2017:4761-4772.
40. Sun M X, Li F, Zhang J. Multi-modality deep network for cold-start recommendation. Big Data and Cognitive Computing. 2018; 2(1):7-21.
41. Dunham M E, Kong K A, McWhorter A J, Adkins L K. Optical biopsy: automated classification of airway endoscopic findings using a convolutional neural network. Laryngoscope: 2020. doi: 10.1002/lary.28708.
42. Rangan S R S. A new human cell line (FaDu) from a hypopharyngeal carcinoma. Cancer. 1972; 29(1): 117-121.

### Example 6—Machine-Learning-Assisted Spontaneous Raman Spectroscopy for the Diagnosis of Human Laryngeal Cancer

### Abstract

Early detection of laryngeal cancer significantly increases survival rates, permits more conservative larynx sparing treatments, and reduces healthcare costs. Laryngoscopy is the current standard for the initial detection and surveillance of laryngeal cancer but relies on visual evaluation for interpretation. A non-invasive optical form of biopsy for laryngeal carcinoma can improve early detection, allow more accurate surveillance for recurrence, and improve intraoperative margin control.

We have designed and evaluated a Raman spectroscopy system for rapid intraoperative detection of human laryngeal carcinoma. Our spectral analysis methods include principal component analysis (PCA), random forest (RF), and one-dimensional convolutional neural network (CNN) methods.

In an IRB approved protocol, we measured the Raman spectra from 207 normal and 500 tumor sites collected from 10 laryngeal cancer surgical specimens (2 total laryngectomies and 8 endoscopic laryngeal cancer resections). RF analysis averaged an overall accuracy of 90.5%, a sensitivity of 88.2%, and a specificity of 92.8% in 10 trials. The 1D CNN can be performed with a 96.1% average accuracy, 95.2% sensitivity, and 96.9% specificity in 50 trials. In predicting the first three principal components (PCs) of normal and tumor data, both RF and CNN can perform well except for the tumor PC2.

This is the first study using CNN-assisted Raman spectroscopy to identify human laryngeal cancer tissues. While convolutional neural networks can be successful in various clinical applications, human cancer classification has been challenging. Our Raman spectroscopy feature extraction approach has not been reported for human cancer diagnosis. Raman spectroscopy, assisted by machine learning (ML) methods, can have application as an intraoperative, non-invasive tool for the rapid diagnosis of laryngeal cancer, including margin detection.

### Introduction

The American Cancer Society estimates that there are 13,000 new cases of laryngeal cancer diagnosed each year [1], [21]. Approximately 3700 patients with laryngeal cancer die annually. Early diagnosis and precise control of disease margins are essential for the surgical management of laryngeal carcinoma. important visual features related to malignancy can be missed on routine laryngoscopy and multiple biopsies can be necessary for diagnosis.

Raman spectroscopy exploits the inelastic scattering of incident monochromatic light exhibited by many substances. Complex biological molecules, including proteins, nucleic acids and lipids have distinct Raman spectral signatures that have been well characterized in the laboratory [3]. Recent developments in Raman spectroscopy technology have enabled investigators to detect variations in the structure and concentration of biomolecules in tissues, including biochemical markers associated with neoplasia [4].

Principal component analysis (PCA) is a dimension reduction algorithm for data processing. PCA is a useful technique for increasing the interpretability of large datasets while minimizing information loss. Previously, PCA for Raman spectra analysis was used to reduce complex, multipeak spectra to 2 or 3 principal components. Supervised machine learning algorithms, with or without PCA, can be used for automating Raman spectra interpretation.

Random forests are a type of supervised machine learning based on decision tree generation for solving classification or regression problems. The algorithm trains multiple decision trees to predict labeled outcomes. During classification, the model traverses all the decision trees and outputs the class reached by most trees. In 2009 Teh, et al. evaluated RF analysis of laryngeal tissue Raman spectra and reported a diagnostic sensitivity of 88.0% and specificity of 91.4% for laryngeal malignancy identification [5].

Neural networks are a form of AI comprising multiple units (neurons) arranged in layers that calculate the relationship between their inputs and outputs based on a set of parameters. Neural networks are a focus of research on machine learning and artificial intelligence [6]. Convolutional neural networks extend the neural network concept with the addition of specialized layers for pattern recognition [7, 8]. Two-dimensional CNNs have been studied in computer vision and image processing research. Similarly, 1D CNNs can be implemented to interpret spectral data. In 2019 Dong, et al. used 1D CNN analysis of Raman spectra to discriminate human from animal blood [9]. There are no reports of CNN enabled Raman spectroscopy used to classify tissue specimens.

We combined PCA with RF and CNN to automate the interpretation of Raman spectra of ex-vivo laryngeal cancer resection specimens. We evaluated this approach to distinguishing laryngeal carcinoma from cancer free margins.

### Materials and Methods

### Experimental Setup and Instrumentation (FIG. 12)

We designed a Raman spectroscopy system comprising a Raman probe (RPS785, InPhotonics, Inc) connected to a 2 mm optical fiber, a 785 nm diode laser source (Turnkey Raman Lasers-785 Series, Ocean Optics, Inc), and a Raman spectrometer with CCD detector (QE Pro; Ocean Optics, Inc). The system was interfaced with a laptop computer for data collection and assembled on a cart for portability. An enclosure was used to shield the specimen and probe during the ex-vivo tissue study. For the tissue evaluations following laryngeal cancer resection, the system was deployed in a non-sterile workspace adjacent to the operating room where the procedures were performed.

### Raman Spectroscopy Evaluation of Resected Laryngeal Cancer Specimens

All patients consented to the study and all the procedures followed human ethical guidelines.

We collected Raman spectroscopic data from specimens taken from patients undergoing resections for histologically confirmed laryngeal carcinoma. Based on evaluations of the system on resected tissue, we determined the ideal incident laser parameters. The laser intensity was set to 160 mW and spectra were sampled with 3-second exposures. For each sample site, we recorded 7-10 spectra.

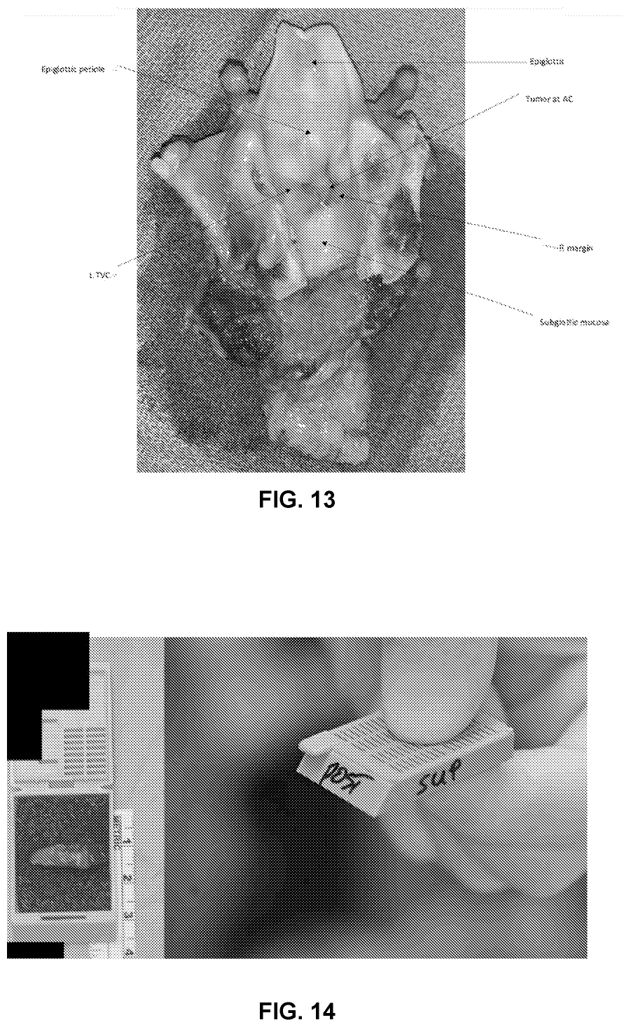

Ten laryngeal cancer specimens were studied, including two total laryngectomy (FIG. 13) cases and eight transoral microsurgical laser resections. All specimens were oriented and labeled by the senior surgeon, consistent with existing surgical specimen processing protocol. The endoscopic resection specimens were placed in cassettes to maintain orientation with labeled margins (FIG. 14).

For each specimen, the engineering team recorded multiple Raman spectra from the labeled margins and central tumor bed. Findings were compared to the final histopathology results. In all cases, the consulting pathologist was blinded to the spectroscopy findings.

### Data Processing and Principal Component Analysis (PCA)

The recorded Raman data were preprocessed prior to analysis [10], [11]. The signal processing protocol was coded using MATLAB (R2018a; MathWorks Inc, Natick, Mass) and included 1) autofluorescence removal with baseline subtraction with asymmetric least squares smoothing [12], 2) Savitzky-Golay filter smoothing and 3) signal normalization.

The mean Raman spectra were calculated from the labeled, preprocessed data for cancer and non-cancer tissue. Principal component analysis was used for dimensional reduction and coded in Python and sci-kit-learn [14]. The sample size was set to the number of components with the singular value decomposition (SVT) solver set to auto. The first three components were calculated (PC1, PC2, PC3) with specific peak marking. The RF and CNN models were also trained and tested with the PCA data to determine the predictive value of machine learning with the Raman PCA datasets.

### Machine Learning Methods

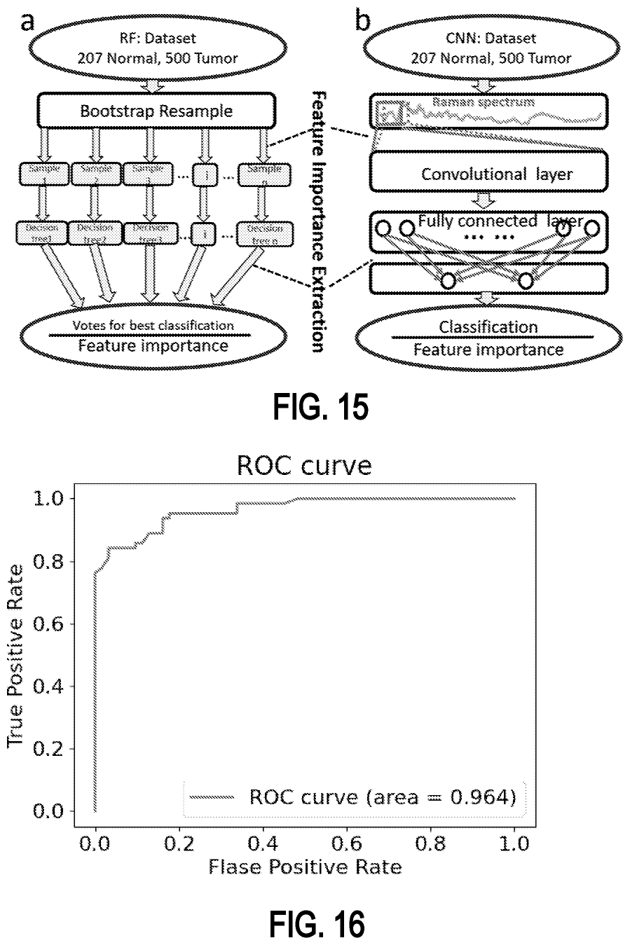

Our random forest model was developed with Python and sci-kit-learn. The RF model architecture is shown in FIG. 15 panel a. The CNN model architecture comprised convolutional layer (32 channels) and one fully connected layer (FIG. 15 panel b) and was developed with Python and Pytorch [15]. The CNN model had a kernel size of 5 and stride of 2.

For both models, we identified the Raman spectral features corresponding to cancer and non-cancer tissues by examining the trained model weights for individual peaks (RN) and wavenumber ranges (CNN) The features were ranked in order of decreasing magnitude.

### Non-Limiting Exemplary Results

A total of 500 Raman spectra were recorded from histologically confirmed cancer positive biopsy sites and 207 confirmed negative margins. Discrete spectra were recorded from 509 cm⁻¹ to 3978 cm⁻¹ across 440 wave numbers. For classifier training, we randomly selected 207 of the cancer spectra to create a balanced dataset totaling 414 recordings.

### Random Forest Classification of Laryngeal Carcinoma Resection Specimens

RF classification was assessed using 10-fold cross validation with a 90% training and 10% testing split of the combined dataset. The average accuracy was 90.5%. Sensitivity and specificity were 88.2% and 92.8% respectively. The RF-assisted receiver operating characteristic (ROC) curve is shown in FIG. 16. The area under the curve (AOC) was 0.964.

The averaged weights calculated for branches in the RF model decision trees corresponding to individual Raman peaks are shown in Table 1. The 24 highest weights are shown.

TABLE 1
Random forest model feature weights. The 3756 cm⁻¹ peak was
the most weighted feature The RF model weighted 12 peaks greater
than 1%. The sum of the highest 24 feature weight was 0.259.

| Wavenumber(cm−1) | Weight | Wavenumber(cm−1) | Weight |
|---|---|---|---|
| 3756 | 0.0184 | 1573 | 0.0099 |
| 1536 | 0.0155 | 3570 | 0.0098 |
| 2381 | 0.0150 | 1592 | 0.0095 |
| 1601 | 0.0133 | 2983 | 0.0088 |
| 1527 | 0.0129 | 1564 | 0.0086 |
| 1583 | 0.0126 | 2776 | 0.0086 |
| 1545 | 0.0124 | 1647 | 0.0085 |
| 1638 | 0.0117 | 3509 | 0.0084 |
| 3761 | 0.0114 | 1384 | 0.0084 |
| 2783 | 0.0109 | 1365 | 0.0083 |
| 1374 | 0.0108 | 2990 | 0.0076 |
| 2920 | 0.0107 | 911 | 0.0074 |

### Convolutional Neural Network Classification of Laryngeal Carcinoma Resection Specimens

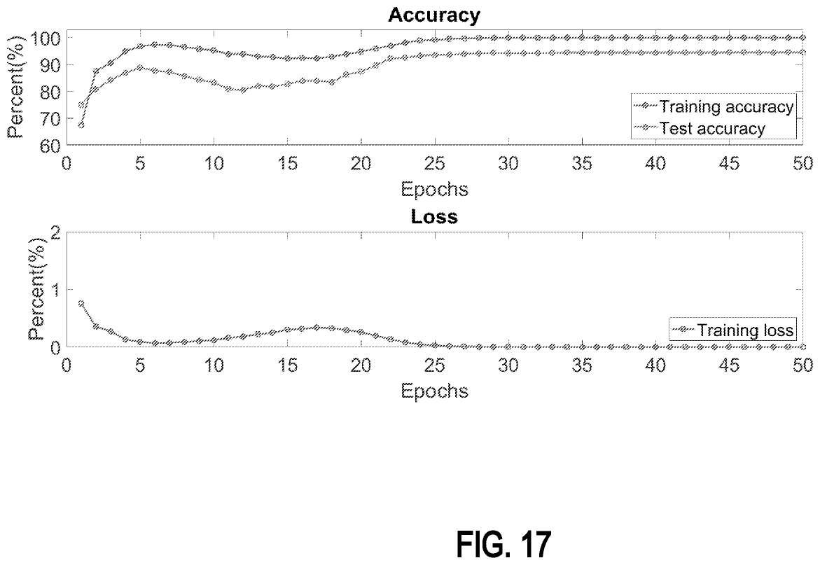

For CNN training, a 90%/10% random data allocation was used for 50 epochs/run. Convergence was noted at 30 epochs (FIG. 17). Results were averaged over 50 runs.

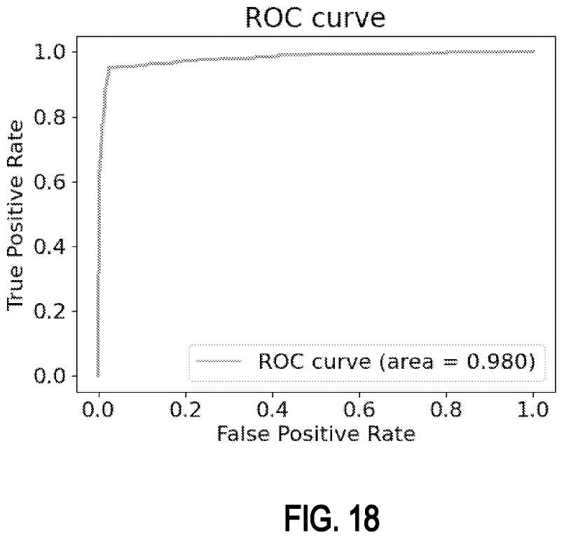

Overall accuracy was 96.1%. Sensitivity and specificity were, 95.2%, and 96.9%, respectively. The CNN-assisted ROC curve is shown in FIG. 18. The AOC was 0.980.

Table 2 shows the top 26 features by weight in the CNN model, corresponding to spectral wavenumber ranges most important for the binary cancer classifier. The weights were extracted and summed from 32 mappings in the linear layer. The most significant range was between 3744 cm⁻¹-3779 cm⁻¹, consistent with the 3756 cm⁻¹ and 3761 cm⁻¹ peak weights in the RF model. The ranges of 921 cm⁻¹-963 cm⁻¹ and 1461 cm⁻¹-1498 cm⁻¹ in the fingerprint region, both had regression weights approximately equal to 1, consistent with existing empirical data [16], [17].

TABLE 2
Features importance weights for CNN classification
of laryngeal cancer (Ranges)

| Wavenumber | Regression | Wavenumber | Regression |
|---|---|---|---|
| (cm−1) | weight | (cm−1) | weight |
| 921-963 | 1.024 | 2887-2906 | 0.219 |
| 1461-1498 | 0.939 | 3552-3577 | 0.216 |
| 3744-3767 | 0.851 | 1046-1087 | 0.214 |
| 3756-3779 | 0.797 | 1188-1228 | 0.203 |
| 1517-1564 | 0.451 | 1647-1684 | 0.192 |
| 2891-2920 | 0.448 | 2990-3018 | 0.175 |
| 2613-2643 | 0.439 | 1208-1247 | 0.175 |
| 3564-3589 | 0.421 | 2962-2990 | 0.167 |
| 1403-1442 | 0.384 | 1536-1573 | 0.167 |
| 2776-2805 | 0.360 | 575-620 | 0.165 |
| 2805-2834 | 0.330 | 2762-2791 | 0.161 |
| 1066-1107 | 0.293 | 773-816 | 0.157 |
| 1845-1880 | 0.250 | 1555-1592 | 0.155 |

Single wavenumber importance was also calculated by averaging the range weight followed by normalization. For example, the average weight corresponding to the first five wavenumbers (w1, w2, w3, w4, and w5), averaged across the 32 convolution mappings, was 0.119, and the average weight corresponding to the second five wavenumbers (w3, w4, w5, w6, and w7, stride=2) was 0.038. Then the weight for the wavenumber w3 could be calculated as the mean of the two, (0.119+0.038)/2=0.0785. The averaged weights were normalized by dividing by the sum of the weights. Table 3 illustrates the 36 largest single wavenumber weights for the CNN model.

TABLE 3
Feature importance weights for CNN classification
of laryngeal cancer (Single wavenumber)

| Wavenumber (cm−1) | Weight | Wavenumber (cm−1) | Weight |
|---|---|---|---|
| 3761 | 0.02085 | 3744 | 0.00774 |
| 3767 | 0.01422 | 3779 | 0.00735 |
| 3756 | 0.01420 | 1527 | 0.00681 |
| 953 | 0.01381 | 2783 | 0.00659 |
| 932 | 0.01345 | 1555 | 0.00653 |
| 1470 | 0.01285 | 3564 | 0.00652 |
| 1489 | 0.01283 | 1077 | 0.00641 |
| 3750 | 0.01122 | 2913 | 0.00628 |
| 3773 | 0.01056 | 2805 | 0.00626 |
| 963 | 0.00965 | 2635 | 0.00620 |
| 942 | 0.00954 | 2620 | 0.00616 |
| 921 | 0.00950 | 2891 | 0.00606 |
| 1498 | 0.00929 | 2906 | 0.00603 |
| 1461 | 0.00929 | 1536 | 0.00595 |
| 1480 | 0.00920 | 1432 | 0.00594 |
| 2899 | 0.00844 | 3583 | 0.00579 |
| 3570 | 0.00806 | 1413 | 0.00571 |
| 1545 | 0.00782 | 3577 | 0.00568 |

### PCA and Combined PCA-ML Analysis of Spontaneous Raman Scattering of Human Laryngeal Cancer

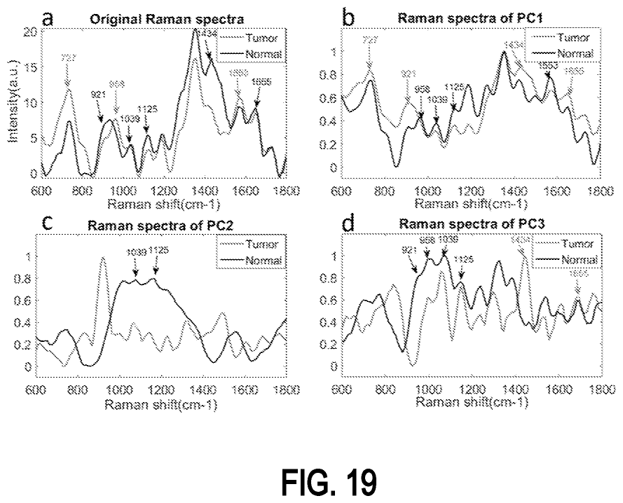

The PCA analysis is summarized in FIG. 19. PC1 plots for laryngeal cancer show distinct Raman signals at 727 cm⁻¹, 958 cm⁻¹, and 1553 cm⁻¹. Non-cancer tissue showed stronger signals at 1125 cm⁻¹, 1434 cm⁻¹, and 1655 cm⁻¹ for the Raman spectra without dimensional reduction. The PC1 plot was similar to the unreduced data. PC2 shows distinct signal differences between cancer and non-cancer tissue at 1039 cm⁻¹ and 1125 cm⁻¹.

Tissue classification with the RF model and PCA data showed 100% sensitivity when predicting normal tissue with all three principal components. Tumor prediction sensitivity was only 50% with PC1 and no tumor spectra were correctly predicted with PC2. Tumor prediction sensitivity was 100% using PC3.

Using the same dataset and the CNN model, prediction accuracy for normal tissue was 100%, 100% and 86% for PC1, PC2 and PC3, respectively. For tumor tissue accuracy was 100%, 0% and 100% for PC1, PC2 and PC3, respectively. PCA-ML sensitivity results are summarized in Table 4.

TABLE 4
Summary of cancer and non-cancer laryngeal resection
tissue classification sensitivities using machine
learning and principal component analysis.

| | |
|---|---|
| RF | CNN |

| | Non-cancer | Cancer | Non-cancer | Cancer |
|---|---|---|---|---|
| PC1 | 100 | 50 | 100 | 100 |
| PC2 | 100 | 0 | 100 | 0 |
| PC3 | 100 | 100 | 86 | 100 |

PC1, PC2, PC3—principal components.
RF—random forest.
CNN—convolutional neural network (1-dimensional).

### Discussion

### Early-Stage Laryngeal Cancer and the Importance of Oncologic Margins

Early detection of laryngeal cancer can increase survival rates and permit more conservative, larynx sparing treatments. It also can also reduce the related length of hospital stay and health care costs [18]. The five-year relative survival rate for stage I glottic carcinoma is 90%. This drops to 74%, 56% and 44% for stages II, Ill and IV, respectively. Lack of locoregional control is the most significant cause of surgical failure.

The past 20 years have seen a trend towards more conservative, larynx sparing surgery for stage I and II laryngeal carcinoma performed through a trans-oral endoscopic approach [19]. Early-stage laryngeal carcinomas can be treatable with larynx sparing endoscopic surgery if adequate margins of resection are achieved. Persistent positive margins can require total laryngectomy and/or radiation therapy that can have otherwise been avoided with complete initial resection[20], [21]. Achieving adequate margins of resection can be challenging [22].

Laryngeal cancer is diagnosed histologically. Frozen section analysis is the current method for intraoperative diagnosis and margin assessment[23], [24]. Lesions can spread in a submucosal plane and multifocal occurrences (“field cancerization”). Identifying the sites for biopsy can be difficult and patients can undergo multiple procedures for biopsy before a diagnosis is confirmed [25]. Without wishing to be bound by theory, a non-invasive, optical form of biopsy for laryngeal carcinoma will improve early detection, permit more accurate surveillance for recurrence, and improve intraoperative margin control.

### Existing Technologies for Real Time Tissue Classification

Optical coherence tomography is a near-infrared interferometry technique. It has resolution and can image on scales ranging from about 10 μm to about 10 mm. It can be used in ophthalmology for retinal imaging. There are also reports of OCT imaging of the larynx and upper airway to measure airway patency. A disadvantage is cost (approximately $75,000 for a retinal imaging system). OCT imaging is deployed with fiberoptic technology that can adapt to existing endoscopes.

NIR optical fluorescence spectroscopy incorporates a fluorescent dye, for example, indocyanine green (ICG) to visualize neoplastic vascularization[27]. In 2017, investigators reported real time NIR-ICG identification of head and neck mucosal lesions[28]. Positive findings correlated with histological malignance with a 89% overall accuracy.

Narrow-band imaging [29], uses blue (415 nm) and green (540 nm) light filtering to enhance the visualization of hemoglobin. It can be useful in identifying lesions with enhanced microvascular patterns including neoplasms. Findings are nonspecific but the technique has shown it can be useful in characterizing neo-angiogenesis in precancerous and cancerous aerodigestive lesions. It is inexpensive and can be deployed in any endoscope. It has proven cost effective in screening endoscopy for colon polyps but success in identifying head and neck lesions has been limited. Other less frequently use modalities for laryngeal tumor imaging include NIR visual imaging with tumor photosensitizing[31] and in-vivo microscopy [32].

### Raman Spectroscopy

When monochromic light from a laser source strikes a substance, photons are absorbed by its surface and reemitted. Most of the reemitted light occurs at the same frequency as the monochromatic source (elastic scattering). Depending on the sample, a portion of the reemitted light will radiate at frequencies above and below the incident frequency (inelastic scattering). Inelastic scattering is dependent upon the molecular structure of the specimen and is termed the Raman effect. One can determine the molecular structure of certain substances using their Raman spectra. Raman signals can be assigned to specific molecular chemical groups and chemical bond vibrational modes. Biomolecules with distinct Raman signals include proteins, nucleic acids, and lipids. Molecular interactions can also display distinctive spectral features[33].

A Raman spectroscopy system can comprise a laser light source, an optical filter to define the incident frequency and a charge couple device (CCD camera) to detect Raman scattering. Raman spectroscopy developers have incorporated several modifications to improve frequency resolution and acquisition times. For example, modular, portable systems have been developed for deployment in the field[34].

The Raman spectra of tissue specimens represent the weighted sum of their macro molecular species and can be tissue specific. Researchers have identified cellular differentiation in several epithelial types using Raman spectroscopy. In situ applications have been reported in brain [35], bladder [36], breast [37], colon [38], and skin neoplasms. Previous studies have also demonstrated a spectral difference between normal tissue, benign lesions, and carcinoma in ex-vivo laryngeal specimens[40], [41].

Lin, et al published a report of in vivo Raman spectroscopy for laryngeal lesions in 2016[42]. They documented spectral discrimination between normal and cancerous tissue during laryngoscopy. Their system used simultaneous 800-1800 cm⁻¹ and 2800-3600 cm⁻¹ wave number analysis. The Raman spectroscopy system included a near infrared diode laser source (785 nm) and a thermo-electric cooled CCD camera. They achieved a diagnostic accuracy of 91.1% (sensitivity—93.3%, specificity—90.1%) for laryngeal cancer. Acquisition times were on the order of milliseconds. They used a 1.8 mm (O.D.) fiberoptic probe with a 1 mm Sapphire ball lens at the distal tip. The Raman probe used during the study was deployed intraoperatively through the operating channel of a flexible laryngoscope. The type and dimensions of the laryngoscope were not specified in their report.

The advantages Raman spectroscopy over other potential tissue imaging modalities include distinct visible spectra for numerous substances, small sample size and nondestructive analysis. Disadvantages of Raman analysis have been its long signal acquisition time and signal-to-noise (SN) reduction due to tissue fluorescence[43]. Recent developments in Raman spectroscopy technology in the near infrared (NIR) spectrum have mitigated fluorescent interference, shortened signal acquisition times, and enabled biomedical applications.

Different components or chemical bonds can result in various peak intensities and wavenumbers of Raman signal. Our results show prominent laryngeal cancer peaks at 727 cm⁻¹ (DNA based Adenine, bending) [44], and 1553 cm⁻¹ (DNA based Guanine, bending). Non-cancer laryngeal tissue shows stronger Raman signals at 1125 cm⁻¹ (proteins, C—N stretching), and 1655 cm⁻¹ (amide I, C═O stretching). The findings are consistent with the known biochemical and cellular transformations in cancerous tissues with increased nucleic acids to protein/lipid ratios [33], [41].

Investigators have documented the importance of DNA glutathione (GSH) as a marker in human head and neck cancer [45], [46]. Glutathione (GSH) is identified in Raman spectra with wavenumbers which can be at ˜1365 cm⁻¹, 1536 cm⁻¹, and ˜1638 cm⁻¹. These identifiers can play a role in cancer vs non-cancer tissue classification. Additional GSH related peaks are identified in the CNN model, including ˜921 cm⁻¹, 932 cm⁻¹, 953 cm⁻¹, ˜963 cm⁻¹, ˜1077 cm⁻¹, ˜1413 cm⁻¹, ˜1461 cm⁻¹, and 1536 cm⁻¹ [47]. The 1536 cm⁻¹ peak is present in both the RF and CNN models.

Raman Spectral Feature Extraction and Classification with Machine Learning Models

Identifying Raman features for tissue classification can be challenging. Studies have designated specific peaks for binary cancer tissue classifiers [48], [49]; however, these designations cannot capture all the feature information available for classification. Diagnostic accuracy can be improved using machine learning trained classifiers capable of capturing a broad range of features. By examining the machine learning feature weights generated during model training[50], we can assign wavenumbers in the Raman spectrum to specific tissues of interest[51].

We evaluated the ability of RF and CNN model to classify cancer vs non-cancer tissue when trained on the original spectra and the first three principal components of the preprocessed data. The accuracy of principal component analysis-linear discriminant analysis has been documented with an overall accuracy of 84.3% when detecting colorectal cancer in ex-vivo samples [52]. Our data indicate improved accuracy using machine learning algorithms, even with a small dataset, compared to empirical peak assignments. In the case of binary classification of cancer vs non-cancer of the larynx, the CNN algorithm outperforms the RF in terms of specificity, sensitivity, and overall accuracy.

### Optical Biopsy of Laryngeal Tissue

Early detection of malignant and premalignant lesions of the larynx can significantly improve larynx preservation and survival rates after surgical treatment. Without wishing to be bound by theory, we can develop a Raman spectroscopy optical biopsy system that can be used in the clinic or operating room. Without wishing to be bound by theory, a design that adapts the Raman probe to both flexible and rigid scopes will allow deployment in the clinic for early detection of premalignant and malignant vocal fold lesions and in the operating room for precise intraoperative margin control during resection. Herein, we describe the non-limiting conceptual design of an endoscopically deployed system and research for detecting laryngeal carcinoma using Raman spectrometry.

### Conclusions

Rapid spectrographic classification of cancer vs non-cancer tissue during surgery for laryngeal carcinoma resection is possible. Raman spectroscopy integrated with machine learning can enable system designers to automate the classification process; the convolutional neural network model can be a ML approach. Principal component analysis provides insight into features in machine learning algorithms used to classify laryngeal cancer resection specimens. Without wishing to be bound by theory, we can rapidly diagnose and define the margins of laryngeal carcinoma.

### REFERENCES CITED IN THIS EXAMPLE

[1] Cohen S M, et al. Direct health care cost of laryngeal diseases and disorders. Laryngoscope. 2012; 122(7): 1582-1588.
[2] Siegel R L, Miller K D, Jemal A. Cancer statistics. CA Cancer J Clin. 2020. 2020; 70(1):7-30.
[3] Rostron P, Gaber S, Gaber D. Raman spectroscopy, review. International Journal of Engineering and Technical Research (IJETR). 2016; 6(1):2321-0869.
[4] Mahadevan-Jansen A, Richards-Kortum R. Raman spectroscopy for cancer detection: a review. Journal of Biomedical Optics1. 1997; 6(1):31-70.
[5] Teh S K, Zheng W, Lau D P, Z. Huang Z. Spectroscopic diagnosis of laryngeal carcinoma using near-infrared Raman spectroscopy and random recursive partitioning ensemble techniques. Analyst. 2009; 134:1232-1239.
[6] Abiodun O I, Jantan A, Omolara A E, et al. State-of-the-art in artificial neural network applications: A survey. Heliyon. 2018; 4(11):e00938. doi: 10.1016/j.heliyon.2018.e00938.
[7] A, Acharya K, Samanta A. A review of convolutional neural networks. 2020 International Conference on Emerging Trends in Information Technology and Engineering. 2020; 2020:1-5. doi: 10.1109/ic-ETITE47903.2020.049.
[8] Y, Shelhamer E, Donahue J, et al. Caffe: Convolutional architecture for fast feature embedding. Proceedings of the ACM International Conference on Multimedia—MM 14. 2014.
[9] J, Hong M, Xu Y, Zheng X. A practical convolutional neural network model for discriminating Raman spectra of human and animal blood. Journal of Chemometrics. 2019; 33(11): e3184. doi: 10.1002/cem.3184.
[10] Mazet V, Carteret C, Brie D, et al. Background removal from spectra by designing and minimizing a non-quadratic cost function. Chemometrics & Intelligent Laboratory Systems. 2005; 76(2): 121-133.
[11] Eliana C, Florian K, Clara S, et al. Evaluation of Shifted Excitation Raman Difference Spectroscopy and Comparison to Computational Background Correction Methods Applied to Biochemical Raman Spectra. Sensors. 2017; 17(8): 1724-1724.
[12] He S, Zhang W, Liu L, et al. Baseline correction for Raman spectra using an improved asymmetric least squares method. Anal. Methods. 2014; 6:4402-4407.
[13] Barton S J, Ward T E, Hennelly B M. Algorithm for optimal denoising of Raman spectra. Anal. Methods. 2018; 10:3759-3769.
[14] Pedregosa G V F, Gramfort A, Michel V, et al. Scikit-learn: Machine Learning in Python. Journal of Machine Learning Research. 2011; 12:2825-2830.
[15] Paszke A, Gross S, Massa F, et al. PyTorch: An Imperative Style, High-Performance Deep Learning Library. Advances in Neural Information Processing Systems. 2019; 32:8024-8035. http://papers.neurips.cc/paper/9015-pytorch-an-imperative-style-high-performance-deep-learning-library.pdf
[16] Chen B C, Sung J, Lim S H. Chemical imaging with frequency modulation coherent anti-Stokes Raman scattering microscopy at the vibrational fingerprint region. J Phys Chem B. 2010; 114(50): 16871-80. doi: 10.1021/jp104553s.
[17] Ranjan R, Ferrara M A, Sirleto L. Extending Femtosecond Stimulated Raman Microscopy Toward Silent and Fingerprint Region of Biomolecules. 2020 22nd International Conference on Transparent Optical Networks (ICTON). 2020:1-4. doi: 10.1109/ICTON51198.2020.9203024.
[18] Arnold D J, Funk G F, Karnell L H, et al. Laryngeal cancer cost analysis: association of case-mix and treatment characteristics with medical charges. Laryngoscope. 2000; 110:1-7.
[19] Hoffman H T, Porter K, Karnell L H, et al. Laryngeal cancer in the United States: changes in demographics, patterns of care, and survival. Laryngoscope. 2006; 116(Suppl. 111):1-13.
[20] Hanna J, Brauer P R, Morse E, Mehra S. Margins in laryngeal squamous cell carcinoma treated with transoral laser microsurgery: a national database study. Otolaryngology—Head and Neck Surgery. 2019; 161(6): 986-992.
[21] Fiz I, Mazzola F, Fiz F, et al. Impact of close and positive margins in transoral laser microsurgery for Tis-T2 glottic cancer. Front Oncol. 2017; 7:245. doi:10.3389/fonc.2017.00245.
[22] Hamman J, Howe C L, Borgstrom M, et al. Impact of close margins in head and neck mucosal squamous cell carcinoma: a systematic review. Laryngoscope. 2021; 00:1-15. doi: 10.1002/lary.29690.
[23] DiNardo L J, Lin J, Karageorge L S, Powers C N. Accuracy, utility, and cost of frozen section margins in head and neck cancer surgery. Laryngoscope. 2000; 110(10 Pt 1):1773-6.
[24] Jaafar H. Intra-operative frozen section consultation: concepts, applications and limitations. Malays J Med Sci. 2006; 13(1):4-12.
[25] von Stulpnagel B, Hagen R, Olzowy B, Witt G, Pau H W, Just T. Comparative Study between the Surgeon's Intraoperative Evaluation and Histopathology for Diagnosis of Laryngeal Lesions. Int Sch Res Notices. 2014; 2014:635251. doi:10.1155/2014/635251.
[26] Gora M J, Suter M J, Tearney G J, Li X. Endoscopic optical coherence tomography: technologies and clinical applications [Invited]. Biomed Opt Express. 2017; 8(5):2405-2444. doi: 10.1364/BOE.8.002405.
[27] Haustein E, Schwille P. Trends in fluorescence imaging and related techniques to unravel biological information. HFSP J. 2007 September; 1(3):169-180.
[28] Schmidt F, Dittberner A, Koscielny S, et al. Feasibility of real-time near-infrared indocyanine green fluorescence endoscopy for the evaluation of mucosal head and neck lesions. Head Neck. 2017; 39(2):234-240. doi: 10.1002/hed.24570.
[29] Piazza C, Del Bon F, Peretti G, and Nicolai P. Narrow band imaging in endoscopic evaluation of the larynx. Curr Opin Otolaryngol Head Neck Surg. 2012; 20:472-476.
[30] Zabrodsky M, Lukes P, Lukesova E, et al. The role of narrow band imaging in the detection of recurrent laryngeal and hypopharyngeal cancer after curative radiotherapy. Biomed Res Int. 2014; 2014:175398. doi: 10.1155/2014/175398.
[31] Zhou X, Li H, Shi C, et al. An APN-activated NIR photosensitizer for cancer photodynamic therapy and fluorescence imaging. Biomaterials. 2020; 253:120089. doi: 10.1016/j.biomaterials.2020.120089.
[32] Cikojevic D, Gluncic I, Pesutic-Pisac V. Comparison of contact endoscopy and frozen section histopathology in the intra-operative diagnosis of laryngeal pathology. J Laryngol Otol. 2008; 122(8):836-9. doi: 10.1017/S0022215107000539.
[33] Naber S P. Molecular pathology—detection of neoplasia. NEJM. 1994; 331(22): 1508-1510. Stone N, Stavroulaki P, Kendall C, et al. Raman spectroscopy for early detection of laryngeal malignancy: Preliminary results. Laryngoscope. 2000; 110(10):1756-1763.
[34] Colomban, P. The on-site/remote Raman analysis with mobile instruments: a review of drawbacks and success in cultural heritage studies and other associated fields. Journal of Raman Spectroscopy. 2012; 43:1529-1535.
[35] Jermyn M, Mok K, Mercier J, et al. Intraoperative brain cancer detection with Raman spectroscopy in humans. Sci Transl Med. 2015; 7:274ra19.
[36] Li S, Li L, Zeng Q, et al. Characterization and noninvasive diagnosis of bladder cancer with serum surface enhanced Raman spectroscopy and genetic algorithms. Sci. Rep. 2015; 5:1-7.
[37] Haka A S, Volynskaya Z, Gardecki J A, et al. Diagnosing breast cancer using Raman spectroscopy: prospective analysis. J Biomed Opt. 2009; 14(5):054023 1-8. doi: 10.1117/1.3247154.
[38] Liu W, Sun Z, Chen J, Jing C. Raman spectroscopy in colorectal cancer diagnostics: comparison of PCA-LDA and PLS-D A models. Journal of Spectroscopy. 2016. doi: 10.1155/2016/1603609.
[39] Zhao J, Lui H, McLean D I, Zeng H. Real-time Raman spectroscopy for non-invasive skin cancer detection—preliminary results. Conf Proc IEEE Eng Med Biol Soc. 2008; 2008:3107-9.
[40] Lau D P, Huang Z, Lui H, et al. Raman spectroscopy for optical diagnosis in the larynx: preliminary findings. Lasers in Surgery and Medicine. 2005; 37:192-200.
[41] Stone N, Stavroulaki P, Kendall C, et al. Raman spectroscopy for early detection of laryngeal malignancy: preliminary results. Laryngoscope. 2000; 110:1756-1763.
[42] Lin K, Zheng W, Lim C M, et al. Real-time in vivo diagnosis of laryngeal carcinoma with rapid fiber-optic Raman spectroscopy. Biomedical Optics Express. 2016; 7(9):3705-3715.
[43] Kuhar N, Sil S, Verma T, Umapathy S. Challenges in application of Raman spectroscopy to biology and material. RSC Adv. 2018; 8:25888-25908.
[44] Haichun L, Zhengdong W, Qiong W, et al. Surface-enhanced Raman spectroscopy for classification of laryngeal cancer and adjacent tissues. Laser Phys. 2019; 29(10):105601. doi: 10.1088/1555-6611/ab3cca.
[45] Zou Y, Li M, Y. Xing, et al. Bioimaging of glutathione with a two-photon fluorescent probe and Its potential application for surgery guide in laryngeal cancer,” ACS sensors. 2020; 5(1):242-249.
[46] Mulder T P, Manni J J, Roelofs H M, et al. Glutathione S-transferases and glutathione in human head and neck cancer,” Carcinogenesis. 1995; 16(3):619-624.
[47] De Gelder J, De Gussem K, Vandenabeele P, Moens L. Reference database of Raman spectra of biological molecules. Journal of Raman Spectroscopy. 2007; 38(9): 1133-1147.
[48] Kaminaka S, Ito T, Yamazaki H, et al. Near-infrared multichannel Raman spectroscopy toward real-time in vivo cancer diagnosis. Journal of Raman Spectroscopy. 2002; 33(7):498-502.
[49] Stone N, Kendall C, Smith J, et al. Raman spectroscopy for identification of epithelial cancers. Faraday Discussions. 2004; 126:41-57.
[50] Selvaraju R R, Cogswell M, Das A, et al. Grad-CAM: Visual explanations from deep networks via gradient-based localization. Int J Comput Vis. 2020; 128:336-359. https://doi.org/10.1007/s11263-019-01228-7.
[51] Fukuhara M, Fujiwara K, Maruyama Y, Itoh H. Feature visualization of Raman spectrum analysis with deep convolutional neural network. Analytica Chimica Acta. 2019; 1087:11-19.
[52] Guo S, Rosch P, Popp J, Bocklitz T. Modified PCA and PLS: Towards a better classification in Raman spectroscopy-based biological applications. Journal of Chemometrics. 2020; 34(4):1-10. doi: 10.1002/cem.3202.

### Example 7—Detection of Pancreatic Cancer by Convolutional-Neural-Network-Assisted Spontaneous Raman Spectroscopy with Critical Feature Visualization

### Abstract

Pancreatic cancer is the deadliest cancer type with a five-year survival rate of less than 9%. Detection of tumor margins plays a role in the success of surgical resection. However, histopathological assessment is time-consuming, expensive, and labor-intensive. We constructed a lab-designed, hand-held Raman spectroscopic system that can enable intraoperative tissue diagnosis using convolutional neural network (CNN) models to distinguish between cancerous and normal pancreatic tissue. To our best knowledge, this is the first reported effort to diagnose pancreatic cancer by CNN-aided spontaneous Raman scattering with a lab-developed system designed for intraoperative applications. Classification based on the original one-dimensional (1D) Raman, two-dimensional (2D) Raman images, and the first principal component (PC1) from the principal component analysis on the 2D image, can all achieve high performance: the testing sensitivity, specificity, and accuracy were over 95%, and the area under the curve approached 0.99. Although CNN models can show success in classification, it can be challenging to visualize the CNN features in these models, which has never been achieved in the Raman spectroscopy application in cancer diagnosis. By studying individual Raman regions and by extracting and visualizing CNN features from max-pooling layers, we identified Raman peaks that can aid in the classification of cancerous and noncancerous tissues. 2D Raman PC1 yielded more peaks for pancreatic cancer identification than that of 1D Raman, as the Raman intensity was amplified by 2D Raman PC1. To our best knowledge, the feature visualization was achieved for the first time in the field of CNN-aided spontaneous Raman spectroscopy for cancer diagnosis. Based on these CNN feature peaks and their frequency at specific wavenumbers, pancreatic cancerous tissue was found to contain more biochemical components related to the protein contents (particularly collagen), whereas normal pancreatic tissue was found to contain more lipids and nucleic acid (particularly deoxyribonucleic acid/ribonucleic acid). Overall, the CNN model in combination with Raman spectroscopy can serve as a useful tool for the extraction of features that can help differentiate pancreatic cancer from a normal pancreas.

### Introduction

Pancreatic cancer is the fourth most prominent cause of cancer deaths in the United States (US). Its five-year survival rate is ˜9% for all stages combined, and only 3% for patients in the advanced stage (Pandya, et al., 2008; Society, 2020). According to recent statistics published by the American Cancer Association, there were 57,600 new cases of pancreatic cancer diagnosed in 2020 alone and 47,050 deaths. Pancreatic cancer has the highest ratio of new death/new cases at 81.68% (Society, 2020). In clinical practice, surgical resection is the primary treatment approach for the removal of pancreatic cancer at an early stage. If surgery fails to resect cancer completely, the resection site is subject to local relapse. Rapid intraoperative differentiation of cancer and normal tissue plays an essential role in achieving complete resection of pancreatic cancer (Yang, et al., 2014).

To date, several approaches have been used for the detection of pancreatic cancer, such as computerized tomography, magnetic resonance imaging, and positron emission tomography. However, these techniques can be time-consuming, expensive, require bulky equipment, and are unsuitable for intraoperative tissue diagnosis with sufficient sensitivity and specificity. A method of tissue diagnosis is the postsurgical histological examination of tumor specimens (Yang, et al., 2014). However, this procedure can take several hours to days to acquire the final diagnostic report, and the accuracy of such an analysis can rely on the sample quality, experience of the pathologist, and medical procedures (Pandya, et al., 2008; Society, 2020).

Raman spectroscopy is a real-time diagnostic tool for analyzing chemical components with advantages including nondestructive examination, no sample preparation, and specificity to the chemical components (Boiret, Rutledge, Gorretta, Ginot, & Roger, 2014; Kourkoumelis, et al., 2015; Pence & Mahadevan-Jansen, 2016). Raman spectroscopy has been used in many fields, including chemistry, food, environmental science, and medicine (Boiret, et al., 2014; He, et al., 2018; Notingher, et al., 2004; Sato-Berru, et al., 2007; Xu, Gao, Han, & Zhao, 2017). Moreover, it has been used to diagnose cancers, such as oral, skin, and breast cancers (Gebrekidan, et al., 2018; Ghosh, et al., 2019; Kourkoumelis, et al., 2015; Manoharan, et al., 1998). To explore the features from the spectral data, some methods for the classification of Raman signals are principal component analysis (PCA), linear discriminant analysis, and support vector machines (SVMs) (Sohn, Lee, & Kim, 2020).

However, the identification of critical Raman features using the above-mentioned methods can be time-consuming and labor-intensive. For example, Raman spectra are processed by PCA in groups. This can result in low efficiency in data usage, and many features cannot be identified manually from other PCA components or scatter plots (Boyaci, et al., 2014; Pandya, et al., 2008; N. Stone, Kendall, Shepherd, Crow, & Barr, 2002; Uy & O'Neill, 2005). In comparison with the conventional methods (e.g., PCA), deep learning does not require manual intervention to extract Raman features, and has now been applied in many medical fields, such as electrocardiogramalysis and tumor segmentation in medical images (Al Rahhal, et al., 2016; Havaei, et al., 2017; Zhao, et al., 2018). The convolutional neural network (CNN) includes a deep learning model, and exhibits several advantages, including the requirement of little prior knowledge or design of explicit features, and a capability to capture inner structures (Fan, Ming, Zeng, Zhang, & Lu, 2019). However CNN models can be difficult to visualize Raman features (peak information) from CNN representations, even most of them can achieve performance in distinguishing different tissue types (Fukuhara, Fujiwara, Maruyama, & Itoh, 2019). The role of recognizing spectral features (particularly peak information) from CNN features is critical in the identification of biomolecular components (Fukuhara, et al., 2019). Our study represents the first effort in this field to extract critical CNN Raman features that aid in cancer diagnosis. In addition, to the best of our knowledge, this is also the first study that adopts CNN-aided spontaneous Raman spectroscopy for pancreatic cancer diagnosis.

Thus far, 1D Raman has been used directly as a dataset for CNN classification (Carvalho, et al., 2017; Lee, Lenferink, Otto, & Offerhaus, 2019; Shao, et al., 2020). However, it can be difficult to perform prefeature extraction from individual Raman data with the use of statistical algorithms such as PCA. Herein, we not only evaluate 1D Raman but also explore the efficiency of 2D Raman images obtained from the dot products of 1D Raman. We also extract and visualize the hidden CNN features from max-pooling layers.

### Materials and Methods

### Animals and Raman Spectroscopy System

In this study, the human CFPAC-1 cell line (ATCC® CRL-1918™, pancreatic ductal adenocarcinoma) was used. Before the injection of the cells in an animal host, tumor cells were grown in Iscove's Modified Dulbecco's Medium (ATCC® 30-2005™) with 10% fetal bovine serum (Neuromics, Edina, Minnesota) at 37° C. and 5% CO₂ in a humidified environment. For the animal model, we used 6-8-week-old female immunocompetent athymic nude Nu/J mice (stock #002019, Jackson Laboratories, Bar Harbor, Maine, USA).

After the CFPAC-1 cells were incubated in media, approximately 2×10⁶ cells were transplanted to the dorsa of the mice by subcutaneous injection. When the size of the tumor was approximately 1 cm, the mice were euthanized, and the entire tumor and normal pancreas were dissected. This study was approved by the Institutional Animal Care and Use Committee of Louisiana State University, and all operations followed the guidelines on animal research.

The Raman spectroscopy system comprised a 785 nm laser source (laser diode, Turnkey Raman Lasers-785 Series, Ocean Optics Inc., Dunedin, Flurida, United States), QE Pro spectrometer (Ocean Optics, Inc), and a Raman probe (RPS785, InPhotonics Inc., Norwood, Massachusetts, United States). When the Raman spectra were acquired, the Raman probe was fixed behind the specimen at a distance of approximately 5 mm. In this study, 20 mice were used. The entire tumor and normal pancreas were extracted from each mouse. Subsequently, 1,305 Raman spectra were collected from the tumor, and 1,224 Raman spectra were collected from the pancreas.

Raman Measurements, Data Processing, and Data Analysis with CNN Models

The measured raw Raman signals can include noise (e.g. from tissue autofluorescence), and the real Raman signals require preprocessing before feature extractions (Pandya, et al., 2008). The procedures of Raman spectral processing can be (1) autofluorescence removal by asymmetric truncated quadratic processing (Mazet, Carteret, Brie, Idier, & Humbert, 2005), (2) background removal, (3) noise removal with the Savitzky-Golay filter (Cordero, et al., 2017), and (4) normalization.

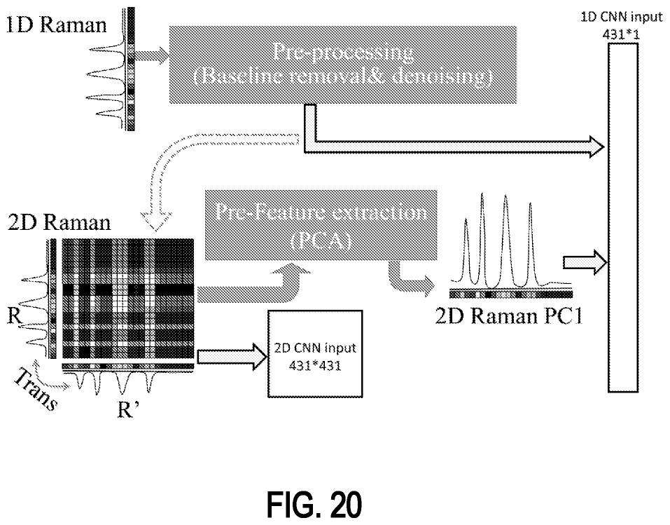

In addition to the original 1D Raman signal, 2D Raman images were also obtained from the dot product of each normalized Raman spectrum and its transpose, as depicted in FIG. 20. CNNs are a model for feature extraction. They enable the extraction of the feature vector from input data similar to the case of PCA (Fukuhara, et al., 2019). To extract the potential features in 2D Raman images, each 2D Raman image was prefeatured by PCA, and the first PCA components (2D Raman PC1) were used as the inputs for CNN models.

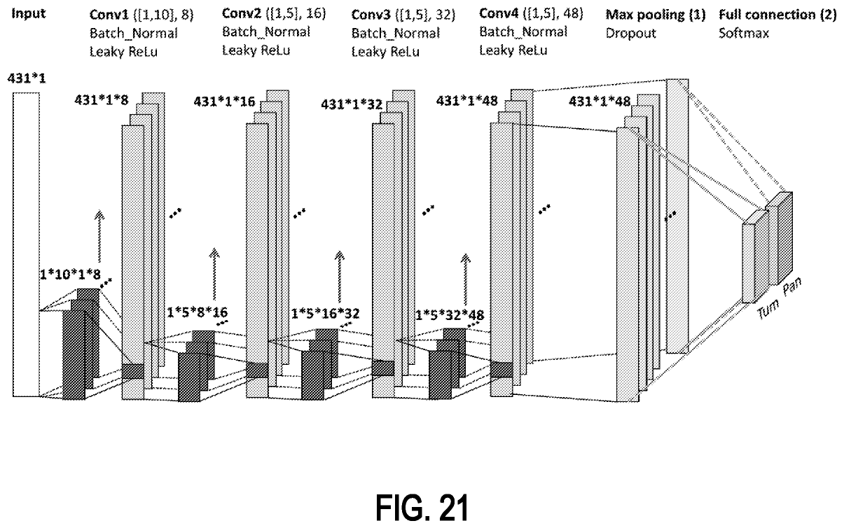

We explored two types of CNN models for the classification task: a 1D CNN model, which was used for 1D Raman signals and the 2D Raman PC1, and a 2D CNN model used for the 2D Raman images. The overall structures of these two types of CNN models were the same and were composed of four convolution layers (Conv), a dropout layer, a max-pooling layer, a full connection layer, and a softmax layer, as illustrated in FIG. 21. Each convolution layer applied convolution to its input, followed by batch normalization (Batch_Normal) and leaky rectified linear unit (ReLu) activation. The filter size of the first convolution layer was set to [1, 10], and the filter size was [1, 5] for the other three subsequent convolution layers. The number of filters was set to 8 for the first convolution layer (Conv1), followed by 16 for the second convolution layer (Conv2), and 32 and 48 for the third and fourth convolution layers, respectively. A five-fold cross-validation was performed to test the CNN models. Sensitivity, specificity, and accuracy were calculated for the evaluation of the model performance.

In the 2D Raman CNN model, the filter sizes were changed to [10, 10] for the Conv1 layers, and [5, 5] for the other three convolution layers. The configurations of the other layers were maintained similar to those of the 1D CNN model. All analyses were conducted with MATLAB (version R2019b, MathWorks Inc, Natick, MA, USA).

### Results

### 1D and 2D Raman Images for Detection of Pancreatic Cancer in a Mouse Model

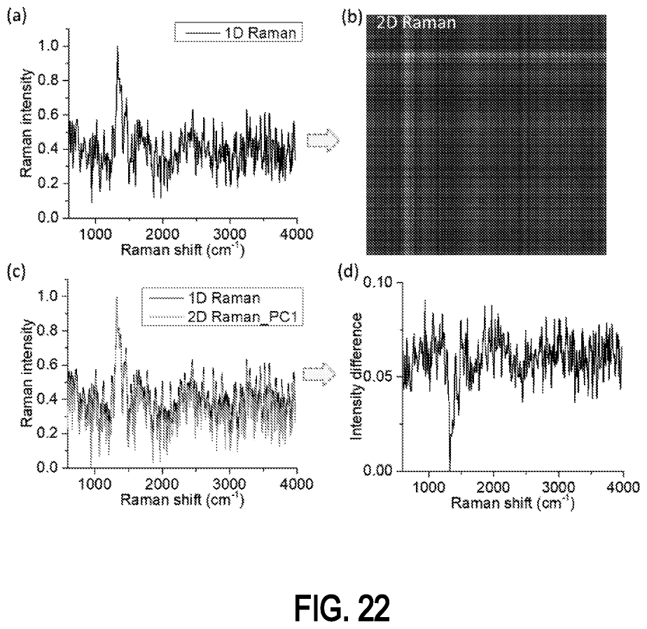

The typical 1D Raman spectrum obtained from the cancerous pancreatic tissue (FIG. 22 panel a) can contain a peak at approximately 1330 cm⁻¹. The corresponding 2D Raman image exhibited a diagonal symmetry (FIG. 22 panel b). The overall trend of the 1D Raman spectrum and the normalized 2D Raman PC1 were similar. However, most of the troughs in the 2D Raman PC1 were relatively lower than those in the 1D Raman curve at the same Raman shift (by 0.08 on average, FIG. 22 panel d). The largest peak remained unchanged during normalization. Thus, the difference between the peaks and troughs of the Raman spectra were larger in the 2D Raman PC1 than in the 1D Raman spectrum (FIG. 22 panel c).

Evaluation of a CNN Model for Detection of Pancreatic Cancer with 1D and 2D Raman Images

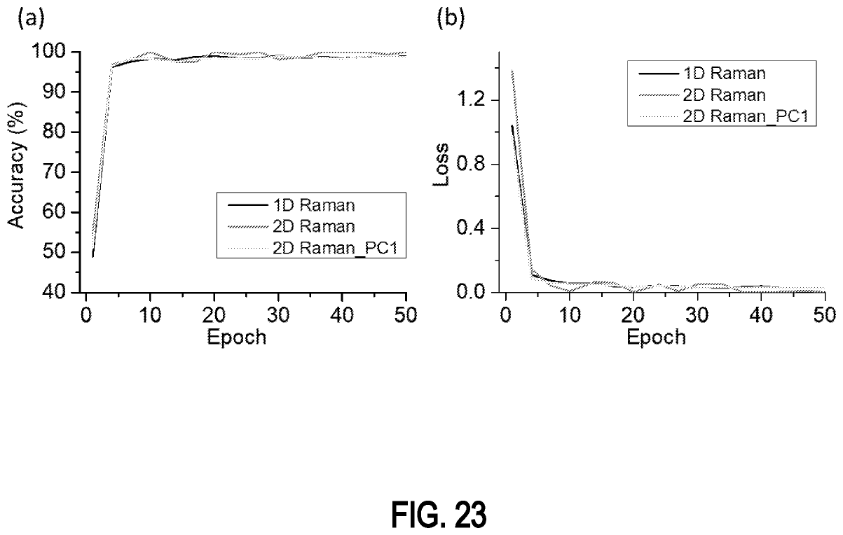

To evaluate the efficiency of the CNN models in identifying cancerous and normal pancreatic tissues, 80% of the Raman data were used for training, 10% of the data were used for validation, and 10% of the data were used for testing. To train the CNN models based on 1D Raman, 2D Raman image, and 2D Raman PC1 (FIG. 23), each training process contained 50 epochs and used a learning rate of 0.001. Both the 1D and 2D CNN models achieved an accuracy >95% only after 5 epochs of training; the training loss became less than 0.05 after 20 epochs for all three models. After 35 epochs of training, the model based on 2D Raman images yielded lower loss rates (<0.005) than those based on 1D Raman (˜0.04) and 2D Raman PC1 (˜0.03). After 35 epochs, the 2D Raman image model yielded a slightly higher accuracy (close to 100%) than those of the 1D Raman model (98.8%) and 2D Raman PC1 model (98.9%).

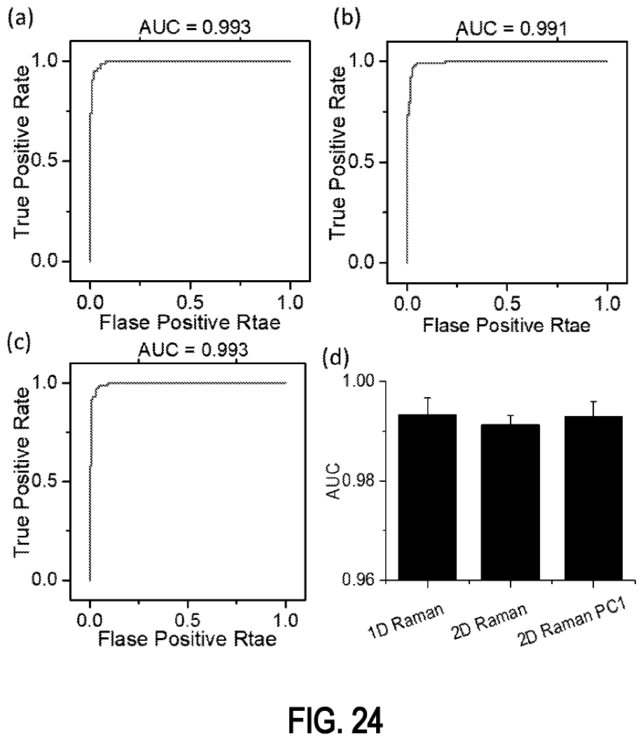

FIG. 24 plots the receiver operating characteristic curves of the 1D and 2D CNN models. All three models achieved high performance in the classification between cancerous and normal pancreatic tissues. The areas under the curve (AUCs) were 0.993=0.003 for the 1D Raman and 2D Raman PC1 models, and 0.991±0.002 for the 2D Raman image model (FIG. 24 panel d).

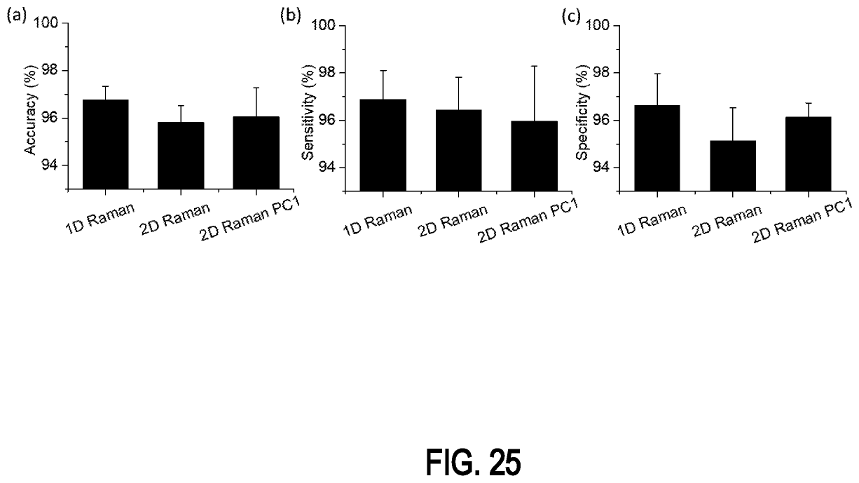

We also gathered statistical measures of the CNN model performance (FIG. 25). All the CNN models demonstrated excellent accuracy, sensitivity, and specificity. The 1D Raman (96.76±0.59%) and 2D Raman PC1 (96.04±1.21%) CNN models yielded a better accuracy than that of the 2D Raman image (95.81±0.71%) CNN model. The 1D Raman (96.88±1.21%) CNN models yielded similar sensitivities to those of the 2D Raman PC1 (95.94±2.33%) and 2D Raman image (96.41±1.41%) CNN models. The 2D Raman PC1 (96.14±0.60%) and the 1D Raman (96.64±1.34%) models exhibited better specificity than that of the 2D Raman image model (95.14±1.38%). Thus, the CNN models can distinguish between cancerous and normal pancreatic tissues through the Raman spectra. All three models exhibited similar and high efficiencies.

Visualization of the CNN Hidden Features from the Max-Pooling Layer

Although CNN was successful in differentiating species by Raman signals, it can be challenging to identify the Raman features that help with this differentiation. A study was conducted to visualize the Raman features from max-pooling layers to differentiate between pharmaceutical compounds and numerically mixed amino acids. (Fukuhara, et al., 2019). Herein, we explored the feasibility of visualization of hidden Raman features in differentiating between cancerous and normal tissues from max-pooling layers.

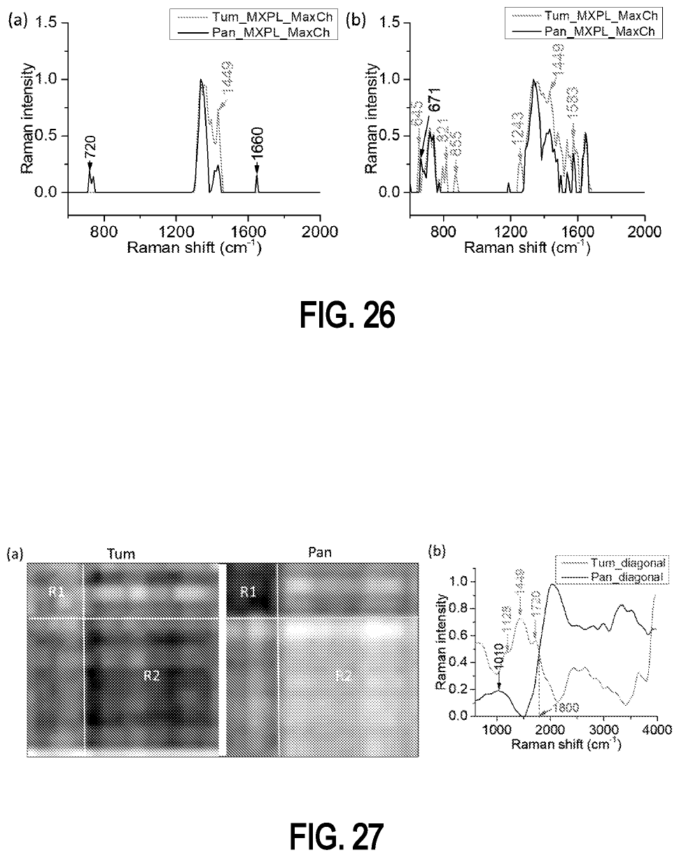

The mean Raman spectra of the 1D Raman and 2D Raman PC1 of the cancerous and normal pancreatic tissues were loaded in their corresponding trained CNN classifiers; the strongest activation channels were then extracted from the max-pooling layers. Given that the sizes of the feature maps in each convolution layer were the same as those of the input features, the visualized CNN features were plotted with the Raman shifts, and some CNN Raman features were visualized from the max-pooling layers. In the 1D Raman CNN model (FIG. 26 panel a), the Raman peaks at 720 and 1660 cm⁻¹ were more significant in the normal pancreas, whereas the peaks at 1449 cm⁻¹ were higher in the cancerous pancreas. In the 2D Raman PC1 CNN model, more peaks can be extracted. The peaks at 645, 821, 855, 1243, 1449, and 1583 cm⁻¹ (FIG. 26 panel b) were only found in pancreatic cancer, whereas the peaks at 671 cm⁻¹ were more significant in the normal pancreas.

The mean 2D Raman images of the cancerous and normal pancreatic tissues were also loaded in the trained 2D CNN classifier. The CNN features were then extracted from the max-pooling layer and reshaped to the original size of the input 2D Raman images. On the reconstructed 2D images, the R1 region was relatively brighter in pancreatic cancer; however, this area was darker for the normal pancreas (FIG. 27 panel a Tum-R1 vs. Pan-R1). In contrast, the R2 region was relatively darker for the cancerous pancreas but brighter in the normal pancreas (FIG. 27 panel a Tum-R2 vs. Pan-R2).

From the plot of the normalized diagonal pixel values, it can be seen that the normal pancreas had a lower intensity than the cancerous pancreas when the Raman shift was lower than 1800 cm⁻¹. This tendency was reversed when the Raman shift was beyond this value. In the wavenumber range of 600-1800 cm⁻¹, the peaks at 1128, 1449, and 1720 cm⁻¹ could be found on the diagonal pixel curve for the cancerous pancreas (red line: Tum_diagnol), and a 1010 cm⁻¹ peak was found on that for the normal pancreas (black line: Pan_diagnol) (FIG. 27 panel b).

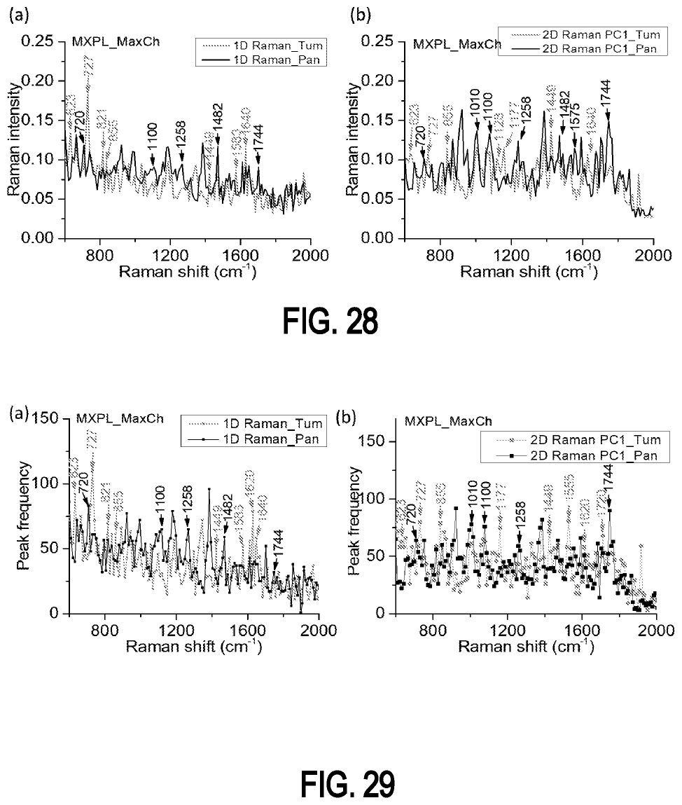

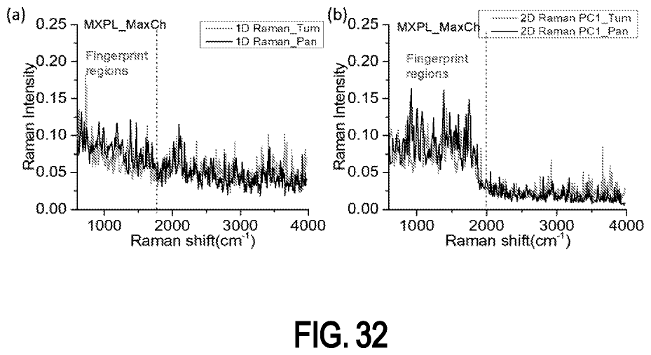

Similar to FIG. 26, all the strongest activation channels of the test dataset (visualized CNN features) were extracted and visualized from the max-pooling layers across 10-fold cross-validation. The Raman spectra with the correct classification were selected and their mean CNN feature curves are depicted in FIG. 28. In 2D Raman PC1 (FIG. 28 panel b), the Raman spectra in the wavenumber range of 600-1800 cm⁻¹ exhibited larger intensities than those beyond 1800 cm⁻¹, whereas 1D Raman exhibited slightly larger Raman intensities in the range of 600-1600 cm⁻¹ (FIG. 28 panel a). This result was more evident in the full wavenumber range (600-3970 cm⁻¹) (FIG. 32). Pancreatic cancer (red line) yielded substantial peaks at 623, 727, 821, 855, 1449, 1583, and 1640 cm⁻¹ in the 1D Raman spectra (FIG. 28 panel a), and the peaks at 623, 727, 855, 1128, 1177, 1449, and 1640 cm⁻¹ in the 2D Raman PC1 (FIG. 28 panel b). The normal pancreas (black line) yielded detectable peaks at 720, 1100, 1258, 1482, and 1744 cm⁻¹ in the 1D Raman spectra, and 720, 1010, 1100, 1258, 1482, 1575, and 1744 cm⁻¹ in the 2D Raman PC1.

We also studied the frequency of Raman peaks of visualized CNN features that appeared across all the tested spectra. In 1D Raman, 623, 727, 821, 855, 1449, 1583, 1620, and 1640 cm⁻¹ can appear for pancreatic cancer, whereas 720, 1100, 1258, 1482, and 1744 cm⁻¹ can be found for the normal pancreas (FIG. 29 panel a). In 2D Raman PC1, peaks with high peak frequency could be observed at 623, 727, 855, 1177, 1449, 1555, 1620, and 1720 cm⁻¹ in pancreatic cancer, and at 720, 1010, 1100, 1258, and 1744 cm⁻¹ in the normal pancreatic tissue (FIG. 29 panel b).

### Screening Frequency Regions Important for Accurate Pancreatic Cancer Detection Using CNN Models

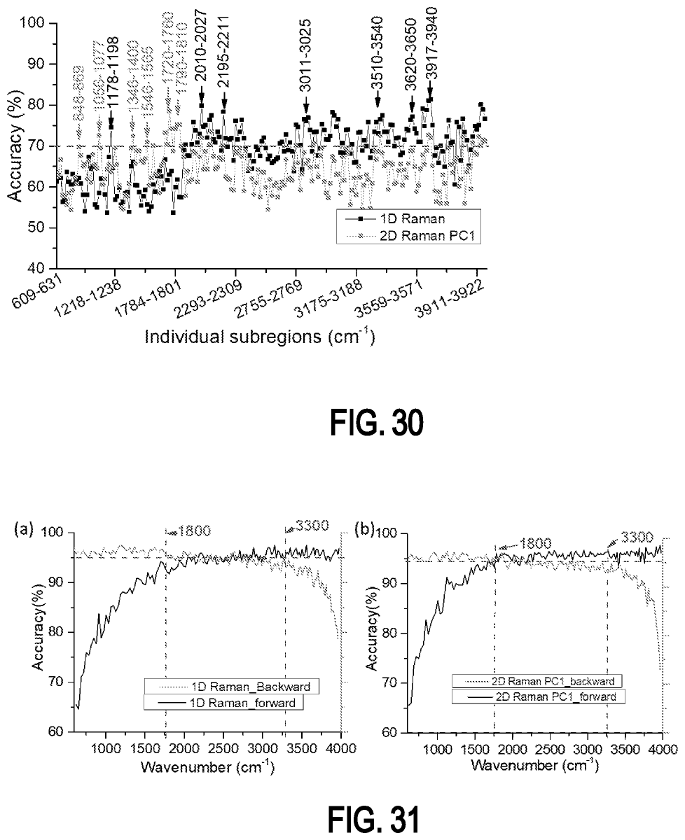

To examine the contribution of each wavelength region to the classification, we divided the full wavenumber range (600-3970 cm⁻¹) into 215 individual subregions, each being 22 cm⁻¹ wide. The subregions were then loaded individually into the 1D CNN model to differentiate between a pancreatic tumor and a normal pancreas. FIG. 30 depicts the testing accuracy of each individual subregion. The subregions of 848-869, 1056-1077, 1346-1400, 1546-1565, 1720-1760, and 1790-1810 cm⁻¹ were found to contribute higher accuracy in classifying tissues using the 2D Raman PC1 CNN model. The subregions of 1178-1198, 2010-2027, 2195-2211, 3011-2025, 3510-3540, 3620-3650, and 3917-3940 cm⁻¹ resulted in higher tissue classification accuracies using the 1D Raman CNN model.

### Discussion

Pancreatic cancer deaths are ranked fourth among all cancer deaths in the US with a low five-year survival rate (<9%). Thus far, there has been no reliable method to rapidly differentiate between cancerous and normal tissues (e.g., <1 min) (Pandya, et al., 2008; Society, 2020). Herein, we designed effective CNN models to differentiate between cancerous and normal pancreatic tissues based on Raman spectroscopy. To further utilize the hidden Raman signal features for the CNN models, we constructed 2D Raman images from the original 1D Raman spectra by the dot products of the Raman spectra and their transposes. Additionally, each 2D Raman image was processed by PCA to generate 2D Raman PC1 data. Compared with the original 1D Raman spectrum, 2D Raman PC1 can enlarge the difference between the maximum and minimum values of the signal. 1D Raman, 2D Raman images, and 2D Raman PC1 were then loaded into 1D or 2D CNN models to classify cancerous and normal pancreatic tissues.

All three methods can be used to classify cancerous and normal pancreatic tissues, where the training accuracies were over 98.8% and the training losses were smaller than 0.05. Compared with 1D Raman and 2D Raman PC1, the model that used 2D Raman images can acquire a higher training accuracy (close to 100%) and a lower training loss (less than 0.005). From the test dataset, it can be seen that all three methods yielded high-testing accuracy (>96%), sensitivity (>95%), specificity (>95%), and precision (>95%) in the identification of cancerous and noncancerous Raman spectra. In the three cases, AUC could reach up to 0.99. Thus, the CNN model with four convolutional layers has high efficiency in classifying cancerous and normal pancreatic tissues.

Currently, gross examination, intraoperative frozen section analysis (IFSA), and postsurgical histopathological examinations are the most common approaches used for the evaluation of pancreatic cancer (Ghosh, et al., 2019; Handgraaf, et al., 2014; Yang, et al., 2014; Zhou, et al., 2012). However, these conventional methods have drawbacks and are incompatible with the accurate intraoperative diagnosis because they are (1) time-consuming (e.g. postsurgical histopathological examinations>20-30 min), (2) subjective to histopathological translation, (3) dependent on tissue preparation (particularly in IFSA), (4) are associated with a limited number of biopsy points, and (5) associated with sampling bias and tissue loss owing to biopsies (Jaafar, 2006; Pandya, et al., 2008; Society, 2020; Vahini, Ramakrishna, Kaza, & Murthy, 2017). Compared with these traditional approaches, Raman signals can be dependent on the changes of chemical compositions (e.g., proteins or nucleic acids) in the biological samples. Raman peak positions and amplitudes contain biochemical information on tissue compositions (Sohn, et al., 2020). Therefore, identifying the Raman peaks or regions that help differentiate cancerous from noncancerous tissues can be crucial in the understanding of the chemical composition changes among various tissues. Many studies have reported that using a CNN model in combination with Raman spectra can serve as a rapid and nondestructive approach to classify chemical species (Fan, et al., 2019; Mazet, et al., 2005; Ralbovsky & Lednev, 2020).

However, visualization and interpretation of the features from the CNN model remains a challenge of this approach. In the field of CNN-aided Raman spectroscopy, there was only one recent report that used simple spectra, generated by the Lorentzian function and white noise, to aid in the feature visualization of amino acids and pharmaceutical compounds mixed with known ratios (Fukuhara, et al., 2019). Herein is the first report in this field to identify the Raman features that can assist in the classification of cancerous and noncancerous tissues. We extracted the maximum activation channels from max-pooling layers to visualize the critical features that are most relevant to the classification by CNN models. In addition, to the best of our knowledge, our effort is also the first CNN-aided spontaneous Raman spectroscopy study to provide intraoperative pancreatic cancer diagnosis.

Based on the accuracy of the screening regions (FIG. 31), it was found that the Raman regions of 600-1800 cm⁻¹ (fingerprint region (Lee, et al., 2019)) are involved in the classification of cancerous and normal pancreatic tissues. This result was further confirmed by the max-pooling features of mean 2D images (FIG. 27) and the mean CNN feature curves of the test dataset (FIG. 28). In the fingerprint region (600-1800 cm⁻¹), the mean 1D Raman and 2D Raman PC1 (FIG. 26) yielded peaks at 645, 821, 855, 1243, 1449, and 1583 cm⁻¹, which were indicative of pancreatic cancer, whereas the 720 and 1660 cm⁻¹ peaks were relevant to a normal pancreas. Thus, both 1D Raman and 2D Raman PC1 helped visualize their unique CNN features for the classification of cancerous and normal pancreatic tissues.

In addition, the CNN features from the entire test dataset provided more feature visualization. 2D Raman PC1 yielded larger peak magnitudes in CNN features than those in 1D Raman. In the CNN features of the test dataset and their high-frequency peaks, the Raman peaks at 623, 727, 821, 855, 1128, 1177, 1449, 1555, 1583, 1620, 1640, and 1720 cm⁻¹ were found in pancreatic cancer, whereas the peaks at 720, 1010, 1100, 1258, 1482, 1575, and 1744 cm⁻¹ were found in the normal pancreas.

Given that Raman peaks are related to chemical components, the visualized CNN features listed above and the high-frequency peaks indicative of pancreatic cancer can be linked with the peaks of 623, 645, 727, 821, 855, 1128, 1177, 1243, 1449, 1555, 1583, 1620, 1640, and 1720 cm⁻¹ that represent protein components, for example collagen contents (Chan, et al., 2006; Cheng, Liu, Liu, & Lin, 2005; Dawson, Rueda, Aparicio, & Caldas, 2013; Frank, McCreery, & Redd, 1995; Frank, Redd, Gansler, & McCreery, 1994; Lau, et al., 2003; Pandya, et al., 2008; Schulz & Baranska, 2007; Talari, 2015). The normal pancreas yields distinct peaks at 671, 720, 1010, 1100, 1258, 1482, 1575, and 1744 cm⁻¹, indicating the components of lipids and nucleic acid (particularly DNA/RNA) (Notingher, et al., 2004; Pandya, et al., 2008; Nicholas Stone, Kendall, Smith, Crow, & Barr, 2004; Talari, 2015). Thus, this method can aid in the analysis of biomolecular tissue components through chemical analysis, and pancreatic cancerous tissue can be rapidly identified by its Raman features.

### Conclusions

Herein, we designed CNN models that could classify cancerous and normal pancreatic tissue based on spontaneous Raman spectra, which was the first such effort in the application of pancreatic cancer diagnosis. The original 1D Raman and the two 2D Raman methods can achieve high performance: testing sensitivity, specificity, and accuracy were higher than 95%, and the AUC could be up to 0.99. Through the screening of increasing regions and individual subregions and the visualization of CNN features, the fingerprint regions (600-1800 cm⁻¹) can be involved in the recognition of pancreatic cancer. With the CNN features extracted from max-pooling layers, we located Raman peaks that were involved in classification of cancerous and normal tissue. From these peaks, we can identify that the cancerous pancreatic tissue contained increased protein content, particularly collagen, whereas the normal pancreas contained more lipids and nucleic acid (particularly DNA/RNA). This was the first effort that visualized features in the field of CNN-aided Raman spectroscopy for cancer diagnosis. Overall, the CNN model, in combination with spontaneous Raman spectroscopy, can serve as a useful tool for the extraction of key features that can aid in the differentiation of pancreatic cancerous tissue from a normal pancreas.

### Supporting Information

Our work has demonstrated the feasibility of pancreatic cancer detection by CNN-assisted spontaneous Raman scattering and the possibility of important Raman feature visualization from CNN models for the first time in this field. The findings will be evaluated further in conjunction with human pancreatic cancer studies. Moreover, our CNN-aided spontaneous Raman spectroscopy, with our lab-designed Raman system, can become a rapid (<30 s), accurate tool for intraoperative tissue diagnosis in pancreatic cancer surgery.

To find the most significant regions that contribute to the accuracy of CNN models, 1D Raman and 2D Raman PC1 datasets were screened in forward (from 600 to 3970 cm⁻¹, ˜16 cm⁻¹/step) and backward (from 3970 to 600 cm⁻¹, ˜ 16 cm⁻¹/step) directions. If one region's range makes more contribution to the classification, the accuracy would increase, for example, at the beginning of the screening.

1D Raman had a rising region from 600 to 1800 cm⁻¹ in the forward screening (FIG. 26), and the testing accuracy remained at around 95% when the wavenumber is larger than 1800 cm⁻¹; this result was similar in the 2D Raman PC1. For the backward screening, 1D Raman had a smaller increase of accuracy (from 80% to 95%) in the regions of 3400-3970 cm⁻¹; the testing accuracy kept at around the 95% level when the wavenumber is lower than 3400-3970 cm⁻¹. For the 2D Raman PC1, the backward screening had a faster testing accuracy (from 65% to 95%) in the regions of 3300-3970 cm⁻¹; this accuracy was still kept at around 95% outside that region. Thus, both 1D Raman and 2D Raman PC1 indicate that 600-1800 cm⁻¹ are the regions that contain useful information for the classification of pancreatic cancer than the region of 3400-3970 cm⁻¹.

### REFERENCES CITED IN THIS EXAMPLE

Al Rahhal, M. M., Bazi, Y., AlHichri, H., Alajlan, N., Melgani, F., & Yager, R. R. (2016). Deep learning approach for active classification of electrocardiogram signals. Information Sciences, 345, 340-354.
Boiret, M., Rutledge, D. N., Gorretta, N., Ginot, Y.-M., & Roger, J.-M. (2014). Application of independent component analysis on Raman images of a pharmaceutical drug product: pure spectra determination and spatial distribution of constituents. Journal of pharmaceutical and biomedical analysis, 90, 78-84.
Boyaci, I. H., Temiz, H. T., Uysal, R. S., Velioglu, H. M., Yadegari, R. J., & Rishkan, M. M. (2014). A novel method for discrimination of beef and horsemeat using Raman spectroscopy. Food Chemistry, 148, 37-41.
Carvalho, L. F. C., Bonnier, F., Tellez, C., Dos Santos, L., O'Callaghan, K., O'Sullivan, J., Soares, L. E. S., Flint, S., Martin, A. A., & Lyng, F. M. (2017). Raman spectroscopic analysis of oral cells in the high wavenumber region. Experimental and Molecular Pathology, 103, 255-262.
Chan, J. W., Taylor, D. S., Zwerdling, T., Lane, S. M., Ihara, K., & Huser, T. (2006). Micro-Raman spectroscopy detects individual neoplastic and normal hematopoietic cells. Biophysical journal, 90, 648-656.
Cheng, W. T., Liu, M. T., Liu, H. N., & Lin, S. Y. (2005). Micro-Raman spectroscopy used to identify and grade human skin pilomatrixoma. Microscopy research and technique, 68, 75-79.
Cordero, E., Korinth, F., Stiebing, C., Krafft, C., Schie, I. W., & Popp, J. (2017). Evaluation of Shifted Excitation Raman Difference Spectroscopy and Comparison to Computational Background Correction Methods Applied to Biochemical Raman Spectra. Sensors (Basel), 17.
Dawson, S. J., Rueda, O. M., Aparicio, S., & Caldas, C. (2013). A new genome-driven integrated classification of breast cancer and its implications. The EMBO journal, 32, 617-628.
Fan, X., Ming, W., Zeng, H., Zhang, Z., & Lu, H. (2019). Deep learning-based component identification for the Raman spectra of mixtures. Analyst, 144, 1789-1798.
Frank, C. J., McCreery, R. L., & Redd, D. C. (1995). Raman spectroscopy of normal and diseased human breast tissues. Anal Chem, 67, 777-783.
Frank, C. J., Redd, D. C., Gansler, T. S., & McCreery, R. L. (1994). Characterization of human breast biopsy specimens with near-IR Raman spectroscopy. Anal Chem, 66, 319-326.
Fukuhara, M., Fujiwara, K., Maruyama, Y., & Itoh, H. (2019). Feature visualization of Raman spectrum analysis with deep convolutional neural network. Anal Chim Acta, 1087, 11-19.
Gebrekidan, M. T., Erber, R., Hartmann, A., Fasching, P. A., Emons, J., Beckmann, M. W., & Braeuer, A. (2018). Breast Tumor Analysis Using Shifted-Excitation Raman Difference Spectroscopy (SERDS). Technology in cancer research & treatment, 17, 1533033818782532.
Ghosh, A., Raha, S., Dey, S., Chatterjee, K., Roy Chowdhury, A., & Barui, A. (2019). Chemometric analysis of integrated FTIR and Raman spectra obtained by non-invasive exfoliative cytology for the screening of oral cancer. Analyst, 144, 1309-1325.
Handgraaf, H. J., Boonstra, M. C., Van Erkel, A. R., Bonsing, B. A., Putter, H., Van De Velde, C. J., Vahrmeijer, A. L., & Mieog, J. S. D. (2014). Current and future intraoperative imaging strategies to increase radical resection rates in pancreatic cancer surgery. BioMed research international, 2014.
Havaei, M., Davy, A., Warde-Farley, D., Biard, A., Courville, A., Bengio, Y., Pal, C., Jodoin, P.-M., & Larochelle, H. (2017). Brain tumor segmentation with deep neural networks. Medical image analysis, 35, 18-31.
He, S., Fang, S., Xie, W., Zhang, P., Li, Z., Zhou, D., Zhang, Z., Guo, J., Du, C., & Du, J. (2018). Assessment of physiological responses and growth phases of different microalgae under environmental changes by Raman spectroscopy with chemometrics. Spectrochimica Acta Part A: Molecular and Biomolecular Spectroscopy, 204, 287-294.
Jaafar, H. (2006). Intra-operative frozen section consultation: concepts, applications and limitations. The Malaysian journal of medical sciences: MJMS, 13, 4.
Kourkoumelis, N., Balatsoukas, I., Moulia, V., Elka, A., Gaitanis, G., & Bassukas, I. D. (2015). Advances in the in Vivo Raman Spectroscopy of Malignant Skin Tumors Using Portable Instrumentation. Int J Mol Sci, 16, 14554-14570.
Lau, D. P., Huang, Z., Lui, H., Man, C. S., Berean, K., Morrison, M. D., & Zeng, H. (2003). Raman spectroscopy for optical diagnosis in normal and cancerous tissue of the nasopharynx—preliminary findings. Lasers in surgery and medicine, 32, 210-214.
Lee, W., Lenferink, A. T. M., Otto, C., & Offerhaus, H. L. (2019). Classifying Raman spectra of extracellular vesicles based on convolutional neural networks for prostate cancer detection. Journal of Raman Spectroscopy, 51, 293-300.
Manoharan, R., Shafer, K., Perelman, L., Wu, J., Chen, K., Deinum, G., Fitzmaurice, M., Myles, J., Crowe, J., Dasari, R. R., & Feld, M. S. (1998). Raman spectroscopy and fluorescence photon migration for breast cancer diagnosis and imaging. Photochem Photobiol, 67, 15-22.
Mazet, V., Carteret, C., Brie, D., Idier, J., & Humbert, B. (2005). Background removal from spectra by designing and minimising a non-quadratic cost function. Chemometrics and intelligent laboratory systems, 76, 121-133.
Notingher, I., Green, C., Dyer, C., Perkins, E., Hopkins, N., Lindsay, C., & Hench, L. L. (2004). Discrimination between ricin and sulphur mustard toxicity in vitro using Raman spectroscopy. Journal of the Royal Society Interface, 1, 79-90.
Pandya, A. K., Serhatkulu, G. K., Cao, A., Kast, R. E., Dai, H., Rabah, R., Poulik, J., Banerjee, S., Naik, R., Adsay, V., Auner, G. W., Klein, M. D., Thakur, J. S., & Sarkar, F. H. (2008). Evaluation of pancreatic cancer with Raman spectroscopy in a mouse model. Pancreas, 36, e1-8.
Pence, I., & Mahadevan-Jansen, A. (2016). Clinical instrumentation and applications of Raman spectroscopy. Chem Soc Rev, 45, 1958-1979.
Ralbovsky, N. M., & Lednev, I. K. (2020). Towards development of a novel universal medical diagnostic method: Raman spectroscopy and machine learning. Chem Soc Rev, 49, 7428-7453.
Sato-Berru, R. Y., Mejia-Uriarte, E. V., Frausto-Reyes, C., Villagran-Muniz, M., S, H. M., & Saniger, J. M. (2007). Application of principal component analysis and Raman spectroscopy in the analysis of polycrystalline BaTiO3 at high pressure. Spectrochim Acta A Mol Biomol Spectrosc, 66, 557-560.
Schulz, H., & Baranska, M. (2007). Identification and quantification of valuable plant substances by IR and Raman spectroscopy. Vibrational Spectroscopy, 43, 13-25.
Shao, X., Zhang, H., Wang, Y., Qian, H., Zhu, Y., Dong, B., Xu, F., Chen, N., Liu, S., Pan, J., & Xue, W. (2020). Deep convolutional neural networks combine Raman spectral signature of serum for prostate cancer bone metastases screening. Nanomedicine, 29, 102245.
Society, A. C. (2020). Cancer Facts & Figures 2020: The Society.
Sohn, W. B., Lee, S. Y., & Kim, S. (2020). Single-layer multiple-kernel-based convolutional neural network for biological Raman spectral analysis. Journal of Raman Spectroscopy, 51, 414-421.
Stone, N., Kendall, C., Shepherd, N., Crow, P., & Barr, H. (2002). Near-infrared Raman spectroscopy for the classification of epithelial pre-cancers and cancers. Journal of Raman Spectroscopy, 33, 564-573.
Stone, N., Kendall, C., Smith, J., Crow, P., & Barr, H. (2004). Raman spectroscopy for identification of epithelial cancers. Faraday discussions, 126, 141-157.
Talari, A. C. S. (2015). Raman spectroscopic analysis to identify chemical changes associated with different subtypes of breast cancer tissue samples. University of Sheffield.
Uy, D., & O'Neill, A. E. (2005). Principal component analysis of Raman spectra from phosphorus-poisoned automotive exhaust-gas catalysts. Journal of Raman Spectroscopy, 36, 988-995.
Vahini, G., Ramakrishna, B., Kaza, S., & Murthy, N. (2017). Intraoperative frozen section-A golden tool for diagnosis of surgical biopsies. Int Clin Pathol J, 4, 00084.
Xu, M.-L., Gao, Y., Han, X. X., & Zhao, B. (2017). Detection of pesticide residues in food using surface-enhanced Raman spectroscopy: a review. Journal of agricultural and food chemistry, 65, 6719-6726.
Yang, L., Sajja, H. K., Cao, Z., Qian, W., Bender, L., Marcus, A. I., Lipowska, M., Wood, W. C., & Wang, Y. A. (2014). uPAR-targeted optical imaging contrasts as theranostic agents for tumor margin detection. Theranostics, 4, 106.
Zhao, X., Wu, Y., Song, G., Li, Z., Zhang, Y., & Fan, Y. (2018). A deep learning model integrating FCNNs and CRFs for brain tumor segmentation. Medical image analysis, 43, 98-111.
Zhou, Y., Liu, C. H., Sun, Y., Pu, Y., Boydston-White, S., Liu, Y., & Alfano, R. R. (2012). Human brain cancer studied by resonance Raman spectroscopy. J Biomed Opt, 17, 116021.
Z. Li, Z. Li, Q. Chen, A. Ramos, J. Zhang, J. P. Boudreaux, R. Thiagarajan, Y. Bren-Mattison, M. E. Dunham, A. J. Mcwhorter, X. Li, J. Feng, Y. Li, S. Yao, J. Xu. (2021). Detection of pancreatic cancer by convolutional-neural-network-assisted spontaneous Raman spectroscopy with critical feature visualization. Neural Networks (IF: 8.050). 144, 455-464.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

1. An endoscopy sheath-probe device, wherein the device comprises a sheath, a Raman probe system, and an endoscope.

2. The device of claim 1, wherein the sheath comprises a Raman probe channel and an endoscope channel.

3. The device of claim 1, wherein the endoscope comprises a rigid endoscope or a flexible endoscope.

4. The device of claim 3, wherein the flexible endoscope comprises a fiberoptic endoscope.

5. The device of claim 1, wherein the Raman probe system comprises a probe, a laser source, an excitation signal filter, a collection filter, a charge couple device detector, a signal collection system, a housing unit, a computer, a display, or a combination thereof.

6. The device of claim 5, wherein the signal collection system comprises a spectrum collection range of about 200 cm⁻¹ to about 4000 cm⁻¹.

7. The device of claim 5, wherein the laser source comprises a wavelength of about 532 nm, about 638 nm, about 785 nm, or about 1064 nm.

8. The device of claim 5, wherein the computer is configured for signal analysis and display capabilities.

9. The device of claim 5, wherein the probe comprises a flexible, fiberoptic probe.

10. The device of claim 5, wherein the probe is configured to control an incidence laser angle.

11. The device of claim 5, wherein the probe comprises an offset distal tip, wherein the offset distal tip is configured to allow contact with a tissue site and control of an optimal incident laser distance.

12. The device of claim 5, wherein the probe is configured to control ambient lighting.

13. The device of claim 5, wherein the excitation signal filter comprises a high-optical density (OD) band-pass filter.

14. The device of claim 5, wherein the collection filter comprises a high-optical density long-pass filter.

15. The device of claim 14, wherein the filter is configured to filter out elastic scattering.

16. The device of claim 5, wherein the probe comprises an outer diameter of up to about 2.5 mm, a length of up to about 2 m, or a combination thereof.

17. The device of claim 5, wherein the probe is configured to permit laser light delivery and signal collection in real time during an endoscopic procedure.

18. The device of claim 17, wherein the endoscopic procedure comprises an upper airway endoscopic procedure.

19. The device of claim 18, wherein the upper airway endoscopic procedure comprises a laryngoscopy.

20. The device of claim 5, wherein the endoscope further comprises an endoscopic light source and the Raman probe system housing unit further comprises a switch, wherein the switch is configured to permit a user to toggle between the endoscopic light source and the laser source.

21. The device of claim 1, wherein the endoscope comprises an arthroscope, a bronchoscope, a colonoscope, a hysteroscope, a laparoscope, a laryngoscope, a mediastinoscope, sigmoidoscope, thoracoscope, ureteroscope, or an endoscope for use in operative endoscopy.

22. The device of claim 21, wherein the endoscopy comprises laryngoscopy.

23. The device of claim 21, wherein the operative endoscopy comprises pancreatic laparoscopy.

24. The device of claim 1, wherein the sheath is disposable.

25. The device of claim 1, wherein the device is configured to permit collection of data in real time during an endoscopic procedure.

26. The device of claim 5, wherein the device is configured to produce a maximum energy exposure of a target tissue, wherein the maximum energy exposure is sufficient to permit collection of Raman data without damaging the tissue.

27. The device of claim 26, wherein the maximum energy exposure is less than about 5 joules/cm².

28. A method of optical biopsy, comprising the use of the device in claim 1 in a subject.

29. The method of claim 28, wherein the method further comprises:
imaging a target tissue with the endoscope;
subjecting the target tissue to Raman spectroscopy using the Raman probe system to generate Raman spectral data;
analyzing the Raman spectral data; and
classifying the target tissue as cancer or non-cancer based up on a peak at any one or more wavenumbers from the Raman spectral data.

30. The method of claim 29, wherein the method further comprises obtaining a dot product of each normalized Raman signal to generate a 2D Raman image.

31. The method of claim 29, wherein the target tissue comprises pancreatic tissue, brain tissue, lung tissue, oral tissue, or laryngeal tissue.

32. The method of claim 29, wherein the cancer comprises pancreatic cancer, laryngeal cancer, or oral cancer.

33. The method of claim 29, wherein the non-cancer comprises non-cancer pancreatic tissue or non-cancer laryngeal tissue.

34. A computer-aided method of diagnostic assessment of a tissue, the method comprising:
receiving Raman spectral input data of the tissue;
subjecting the Raman spectral input data to at least one data analysis model, wherein the at least one data analysis model uses the Raman spectral input data to classify the tissue as cancer tissue or non-cancer tissue.

35. The method of claim 34, wherein the data analysis model comprises principal component (PCA) analysis.

36. The method of claim 34, wherein the data analysis model comprises random forest (RF) analysis.

37. The method of claim 34, wherein the data analysis model comprises convolutional neural network (CNN) methods.

38. The method of claim 34, wherein the Raman spectral input data comprises 1D Raman spectra or 2D Raman spectra.

39. The method of claim 34, wherein the Raman spectral input data comprises a mean Raman spectrum.

40. The method of claim 34, wherein the tissue classification comprises pancreatic cancer tissue or non-cancer pancreatic tissue.

41. The method of claim 34, wherein the tissue classification comprises laryngeal cancer tissue or non-cancer laryngeal tissue.

42. A method of diagnosing cancerous tissues comprising:
positioning patent for the according to a standard of care for a procedure;
deploying the device according to claim 1, wherein deploying the device comprises inserting the device into the patient;
identifying an anatomical tissue of interest;
subjecting the anatomical tissue of interest to Raman spectroscopy; and
classifying the anatomical tissue as cancerous or non-cancerous.
